SEQUENCE LISTING (107 sequences)

SEQ ID NO: 1 - DNA, 42534 bp, Homo sapiens
  misc_feature (1)..(219): Exon at nucleotide positions 1-219
  misc_feature (971)..(1185): Exon at nucleotide positions 971-1185
  misc_feature (1538)..(1670): Exon at nucleotide positions 1538-1670
  misc_feature (1754)..(1757): Exon at nucleotide positions 1754-1757
  misc_feature (3178)..(3238): Exon at nucleotide positions 3178-3238
  misc_feature (3660)..(3804): Exon at nucleotide positions 3660-3804
  misc_feature (5354)..(5464): Exon at nucleotide positions 5354-5464
  misc_feature (6118)..(6233): Exon at nucleotide positions 6118-6233
  misc_feature (8755)..(8887): Exon at nucleotide positions 8755-8887
  misc_feature (11643)..(11718): Exon at nucleotide positions 11643-11718
  misc_feature (12936)..(13031): Exon at nucleotide positions 12936-13031
  misc_feature (14338)..(14470): Exon at nucleotide positions 14338-14470
  misc_feature (16613)..(16724): Exon at nucleotide positions 16613-16724
  misc_feature (21261)..(21299): Exon at nucleotide positions 21261-21299
  misc_feature (22250)..(22352): Exon at nucleotide positions 22250-22352
  misc_feature (23421)..(23586): Poison exon at nucleotide positions 23421-23580
  misc_feature (27244)..(27411): Exon at nucleotide positions 27244-27411
  misc_feature (28257)..(29090): Exon at nucleotide positions 28257-29090
ctgccgcggccccgcctcgccccgtttccggcgcggcccagcgagctcggcaacctcggcgcagcgagcgcgggcggccagccagggccagggggcggtggcggccaaggtccgaccgggtgccagctgttcccagcccccgcctcgggcccgccgccggcgccgccatgggcaagaagcacaagaagcacaaggccgagtggcgctcgtcctacgagggtgaggcggcggcgctttgtgacgcgcggcgggcggggtcccgggcacgggggaggagttccgggcacgcggacgggggtcctgggcaccgggcgaggtcctggccacgagggaggggttctgggcacgcgggagggggtcctgggatttcgggcagggaaccgggcacacgtgaggggtcctgggtaggcgcttacgttcctggtagagggttctgggttcgaggctgcaagcccactcaccccggtcctggagactagggaagcatctgtgctggcttttgtgtggagctcagggagaagctgggctgccctttctcccgcctttccctccctctcccttgctcccgcagcagctccatgggtgtcagcgggcagcacgggcgcctgcaggaagtcaggcgagggtgaaccgaagtgctgggggtttctaggagggagatcgggctccggggttccaggtccctcttaatccatcttcttgaacgagtaatgtgaacaacatcccacagggcctctggtgaggacaccgtggctcgaaagggacctgaagctccctgcggaggggaagggagaacaaggaggcagtgacatgccgccactgtggtcttcccttccgtctttccctgctggttggcagccccgctgtactccttagggaggcctttccctcttagcaccttcacgtctcacctggctgcgtggcgtttgcacctgagcacgcgggtagaactcggtctgtgtctgtgcagattatgccgacaagcccctggagaagcctctaaagctagtcctgaaggtcggaggaagtgaagtgactgaactctcaggatccggccacgactccagttactatgatgacaggtcagaccatgagcgagagaggcacaaagaaaagaaaaagaagaagaagaagaagtccgagaaggagaagcatctggacgatgaggaaagaaggaagcgaaaggtaaaggagcagcggcggtggcccggacgctgcccacgaggagcccgtggggagcctggtcccaggacccaggcagagtgcgcctgcgtggctggaggaggggagggggtgttccgttggtagcaaaacgcgaaccgctgaggcccgcagactcagttccaggatcctgggtgtgctgttcctcagcagaccctgccagggtctctgtgaggagggagcctggcatgaggaggatctcggagggggtttcccttagttctgtccctcccctgagaactgcgaggggattgggcagacctgtccggatcccgcctactcctgcctttctcactcccttgactctgccgcccaggaagagaagaagcggaagcgagagagggagcactgtgacacggagggagaggctgacgactttgatcctgggaagaaggtggaggtggagccgcccccagatcggccagtccgagcgtgccggacacagccaggcaagggtgcacttttagagactctcctggtgttcacagctcatcgttgtaaatgaggggtcggcaccgtgtcctgttgctttggggagaagcatgggagggaggagagctggtatttccagggcctcatcccaggtgtcctctggcctgaggtttcctccggtgtctctgaatgtctagagcaaggcgtggccagttaccactggtgcccttcagttttgttttttaacttgttagctaaggatggcttatatgttttttaagcgttcataaaaagaaacaaaaaggcatctccacagagaccaatcgtatgtgacccgcaaagcctagactgttttttgtctggcctttatgtaaaaagtttgccagccctgggtcttgaagtttggttttgtttgcacttaagacagtattggaacagtagccccatctgggtcagactggtaagagagatgcatgtcagtgaaaacgcaggtaattccccttgggaagaagggacctcgaggcccaaatggtgggtctgaagttgctgaaagtgaccataggctgagacaaaggaagctggaggcctttagtagaatctctgtcgtgggcaccatctgcgaaggatggcatttggctggaatttccagttgacaaggacttgccctctgagttctctttgtccccagccttcctgtgcacaactcacagtgatgaggcatttgtacctaagacaaggggtgtctcttcccagcaactctcagcatccagtggttcctggattatagggaaacagacggctgggctttgggacatgggacatcagggagtggggaggctagtgctgcgatcattcaggccgtcagaaactgtctgaggcctgctggctccactgaaggaaagttctggaaagctggtggccttctgagctcctgtggtcatttggctcttcaggcactttctttttttaatttatttttgaaacatggtcttgctgtgtcgcctaggctggagtgcagtgtcgtgattcactcctgggctcagataaccctgggctcaagcgatcctcccacctcagcctcccaagttgctgggactacaggcacgcaccaccacgccggccatttatttactttttgttgaattttaagggacctcaagtgaaataataatcggtgaattcaggatcaacctttatgtccagagcgtttgttccgtcgtccatttcttgtctagagtagtggtatgagctgaaaacattgctaagtgatcaccacatattgttgatgtaagggctaccggttattacaagagatttttatgactcctgctgtgggggtgccctttctacccggctgagcttggtgggagccaaggctgagtcggatgtatctttatatccccagttctcggtggaacttaaaatgctgtgagacaccagacagacagatactgtgaacttggagctctctaatgaagggataccaaagtcttgtattcaatttttttttccttaaattgtcagccgaaaatgagagcacacctattcagcaactcctggaacacttcctccgccagcttcagaggtaaaccggggcgtttctgaagagctagtgtctgggggggtgtggtgtcatcttttctgaggccttattccccacgccagcctcgtcagctgtgaaacagaccctgcaccttctcttggtgactcccagtccacttcggggtgcgtaggaagagttcatgaactcattagtggctcactctgaagtcagagtagagtgagggaagtcaaggctagagctgggacattgatggccgccaggatgcgttgggggaatttttattatatctgaaatacctatcacaataaattaaaagacgctcgtaacttttttatttttaagaaacaaaattcggtcagttgtatccaatttttggattgtaagagattttgtatcatttagaaatctagatttttccgctttaagatgtctttgctttcagaaaagatccccatggattttttgcttttcctgtcacggatgcaattgctcctggatattcaatgataataaaacatcccatggattttggcaccatgaaagacaaaattgtagctaatgaatacaagtcagttacggaatttaaggtaagttactttcattgcccgaagtaatcgtggcatagttctgtgtattcatgtcttgtgaagacttcattcaccttagatcatttctagtttttgagctttcacaggtgtttctgtgttcggatgatgttttacagacacagcattaaggaaatgattggtcgtgaacgttttctctgtgctttggtctagaaactaggtcaggttgcacgctgttagaagtctgtttcttttctttctaagttactgcgctggcgtaaataggacatgttgtttcaagcagtgaggtgtccggtgtggcacggcttgagactagacatttttaaaaacatacttctttttaattcttagttttgtcttacactaattgagcatttagtttttttagcatagtaagtttttctgtctcctgagtactttgctcttagctctgtttcttgctactggctgtgagaattagcaactgctcattggttttgcaacccaggggaagaaagcaaaacaaatgcttgataaagttggcattagtgtagtttagaaaggcattagatttctctcttaggttccattcccctggggaaagcttgacttttctattttatgcttactgaagggtttgctgactcacagaaacacaacagaacagacttaaaggagagtgggagaaaaggaaactcgatgagaggatcaggcagcgcctgagggtagatgttaagagcactccatgcccgaaagccctctgtattccctgcttcgggccccagtctggccattcctagcaacaaagtgagaaggggagcagcattcactccgtggagtgtccgcagtggagtcttcaggggaggggctccacagcctcgggccgggccctagggctgctggctctggaaccgggacttggggagggtccagatggcacgttctcagttagggtggccggcctccggggtcatgctccagcctgacgttggtactcacctttgagaggcctgtgctgaggaaactggcctctcataataaaatactgatgcttccgtattttcaccataaattacaaaataaggttgtccttttgtggggcaaagtctttttctagtagtttgagggaaccatttggtgacagaattttaaattcgattctgtggagaaattctagagatagaggctatcactgtgccgcacgcctgccttcatcccttgacttgcacttgaaagaatcgggtcgttgcttctcgttcctgtttatgttctggagaaatgcaagaagcgcaggaaacgctttttcctcacacagcatttgtatgtgatgcttagaaagaagttacaggcttggccaactgtttgctgtaatatttttcatgcaagaccaatgtgcaactaagaaaacttctcttaagttcttaaacgttatgtttagatcagagagcccagttgaattgttttctactcgattgtagctttttccttttggtctcttggtagtcatctagagctttgctgttgtctgtggcatttcaaacctgataagtctttcctgttttcttttcaggcagatttcaagctgatgtgtgataatgcaatgacatacaataggccagataccgtgtactacaagttggcgaagaagatccttcacgcaggctttaagatgatgagcaaagtaagaaactcactggcagcagcgagctacggaaagcaggggctctgccccccgtcgcttgtgcccgaagggagcctcacagtgaacacgtgctccatccacccgccaccctcatggtgttgccccctttgctctagggtagacgtccccggccgccctctctgctcatgacctagctgtaacatctcattgagggttggtggaaagttccgcatctcttgatcttaagcctgggtgtcaggtgtcagtcttgctggatggcagccagcattgcgggcttgttttgcgtgactgcctcactgaatccagacgggacatccttgtcttccttggccatcctgagtggcttcccagatctgtgccgtgagccaggctgcatgcatcccacgtaggagaaggtaaaggggaggtgcggcacctgccgcgctctggcggtgggtcaagcccccacaaccacctcccgctgctccgccgtggaaagttagctcttaggccaggtgaggctgtgaccatccccatcccatcccatcgtcacggcaccctggctgtgagggagaggtaaatccctgctgctctgtcctttctgattctaggagcggctgttagctttgaagcgcagcatgtcgtttatgcaggacatggatttttctcagcaggcagctcttttgggcaatgaagatacagctgttgaggaacctgtccctgaagttgtaccagtacaagtagaaactgccaagaaatccaaaaagccgagtagagaagttatcaggtaaaggttctgtggaaaccacatttgcttttggagttgagcaccatagccaggaaagcaggtgccttgtaggggagagtgagtgagtgagagaacggatgtcatgtcacatagacattcttggtgtagcttattctggcaggaggagtgtggccagacagcccctcctgcccaccggccacgtgcactctctgacccaggtgtggtacccaagcccgtccgatcacccctgcccccaactgctagtgactggtggtctctgtctctacacgccgttaagatcattggcccctccctcccctgcacagcgcctgccggcagccaaggctgtgcccttgtcccagactgtgccgcacaagcgggctgactttgggcagccagcattggcagggggcttctttgttgctgtggggcagatgcacactcctgaactgctcgcttctgtgtctgtgtagacgctggagtggagcaggtgcaggcctgtggtcaggaaggtgtggatgctgcgggggaagcaggtgaggactgcaggccacagctccctgtagctctgctccctcccacagggagcagccgacttggctggtccttcagggaaccttcttcctttttttctaaaccccagagtggttttcatgggggctctttgtctcatggtggctgagcattgagattgtatcaaagtccgtatcacacgtcattcctgtagacgctcctccacctatgtgaggtggctgcctgataggaccaaacaaagcgagagtatcgccaggggccttagaggcaccaagagcacttaacatagcctgcagcgggcagaagcgcctggcacagagcctgcttcatcacgtgtcacttgctgagtgcaggccccagagagcgcagtctcaggacatccaccctcatggccgtgtgtctgacagcactgtggctcgcggtcttcccagtatcctgagaggactggcctgttgccatcctggggacagatcagatttgaaggatggtttctgctgaagtgtattgcttttgtgccatcttgtgggaagtcagggaccgtctggagttggtactgaacgtccttgaggcaattgtgatggtcacggccctccattgtttgtttccttctcacatgtgtatgggactcacgtggatttaacaggtggggcggcagcggtgctgggctcggtgcttctgctctgagcagctccctccaaccgtcaaaagggagagtggtcttgagaccagcccctcaggtcctgcagccttggcttaggagacagggtgcgctgcgtggccagagtgtgttgcttggcaacggacgacagtcccctaaggcggtctccccacgtgcgttgcatttcccaccccgctgccgtgtgaacactcactgcgtggttggctggtggctgtgtccggcctcagcccccgactcgagtgtgctgctggccttcaggggtcggggtttcatggccgccctttgccgcctcatggtgactgagcatcgagggggaattgaagttggtgctggtcagcatgctcagctgcggtggggttgtgtcctggcagacccaccccaggttggttttgggtgtctctggtgctgcagcttcacccttgcctgtgggatccaggccctttagatggatccccagctgtcagtctgcaggcaggagagcgtgccgagctcaggcagtcaccttcacctagtgtcacagcagtgcgcatcggtaatgggctcacgcaggtgcctgagtggagcccctccctgcggagctcatggggctgtgactcctgtgtagggttgccaggttgtcactgtaggtcaggcaagggcagggccgctctcatgggcagggcttgtcctgggcctgcacctggccctaggccactcagacccatgcccaggcaagtgcccagggtgggaaggagcccactcgcttgcttctagaaccttccacactccttgggtgctacagcctgtgcccctcagctgttggagaactgcttcaggtggaacctgggaccacttgctgacgtccttgtgtgttctttgtgccacccagtggccacagcagagggggctgctgtccacccagcccagcagcaccagaccacacccgagcagccccgcctatcctgccttgctgtgaaccctgcgtagtcaggggtggcttttccagtcacttttctgatggggcctttaataactaaccagtgtgggcatctgttactttgttgcttttgttgctaaagcactttaactgagtttgcactgcgttgaaaaataggaagtctgtctacttggcttacacttaccctttattttaataaaagtgtcttaatttgtcatttggttagggtgaaaagggatgctttgagacctaaaaggagttaagtatgctctgccagctctgtgtctgagggcagggtcggtagaagctgggttaggtgccgacgcatggggagcaggtgtgcggtggggacgctgcctccaggtgacttggttgtccccacctcacagctgcatgtttgagcctgaagggaatgcctgcagcttgacggacagtaccgcagaggagcacgtgctggcgctggtggagcacgcagctgacgaagctcgggacaggatcaaccggttcctcccaggcggcaaggtagtgctctgtggccaccgagagagggtgccacgtggccccagactctctgtgtgcctctccttctgggttggtgctgtgtgatctagcacagccaccgggatccagaggtcagcgtggggccctgacgggaggtccggatcagacacacataaggctggtctcttcagtcggggtccaggtgcctctggcagggctgccgctggtgtttcatgcatcgtggaggccacgtggtcactgacctcatttccttgatggaggccctgctgcaggctaatcctttctcttggagtgaatgctgacttctgtgtctctgtcactgggtgttgacgtgaggcaccgcccttgttattccagtgggggtgctggtgttggatccgacagatggtccataataggccaccgactgtgttcacgtggctcctgccacagatggtctttgtggtggtggggcccactcagctgaccacgctctcctttctggcaggagacgttctagacccagtgtcttcagtggaaacagagcacaggctgtctgcagtctcaggcttcctagcagccctgataagagaagtagagcaatgcaggtgacattaatccttgcaggtttcatttcacctctatatccaacattttatctcaacatgtagtctgtagaaaacgttaatttgagctttatgggtttttttcatattaagtcttcaaaatgcacatgggatgggatgtaaagaacgctgaagtgggggcatttcagaggtgtctgctgctttttgcctgtgcctgctgggaagttgcagtgctctgtgtggcttgcgtgttgcgaggttgcggtctgcgtggctcacgtgttgggaggtcgtggtctgcgtggctcacgtgttgggaagttgcagtggtctgtatggcttgcgtgttgggaggttgcagtctgtgtggcccgtgtgttgggaggttgtggtctgcgtggcttgtgtgttgggaggttgtggttcacgtggcttgcatgttgggaagttgctgtggtctgcgtggcttatgtgttgggaggttgtggtttgtgtggcttgtgtgttgggaggttgcagtctgcgtggctcgtgtgttgggaagttgctgtggtctctggcttatgtgttgggaagttgctgtggtttgcgtggctcgtgtgttgggaggttgcagtttgcgtggcttgcgtgttgggaagttgctgtggtctgtgtggcttatgtgttgggaggttgtgggtttgtgtggctcaagtgttgggaggttgtggtctgcgtggctcacgtgtggggcggttgcttggctcgcctgttgggagtttgtggtttgcatggcttgtgtgttggaaagttgctgtggtctgtgtggcttatctgttgggaggttgtggtttttgtggcttgcgtgttggggggttgtggtctgtgtggctcacgtgttgggaggttgtggtctgcgtggctcacgtgttgggcagttgtctgcttggctcacgtgttgggaagttgtggtctgcgtggctcatgtgttgggaagttgtttatgtggcttgtgtgctggaggttgtggcctgcgtggctcacgtgttgggcggttgtctgcttggctcatgtgttgggaagttgtggtctgcgtggcttgtgtgttgggaagttgtttatgtggcttgtgtgctggaggttgtggtctacgtggcttgcactgtagttcttttggacagccttgttctgcagtctgtgtttcagccctggaattgccacattctccaagaagccctggtttcttgactagagagccttgtgtagaagccaggacctgggttctgggtgaagccctctgcagatagagctgggagatgcctgttgtgtgtgtctgcaccacatacatggacactgtgcagagggattctgttggcacctgtgtttacatgggttgaaaactgtttttacacggaaacttccagttccctcgtgccccgtgggttccgcccacttcttccctttcagttttgtatctgcgccgtgaggacttgtctcctaatcttcgtatcaactccgtgtcccattgagctgaccttccgcctctcccccagggccctggccatcctgtgggctctgagactttgcatggatccgcggtggggagctgcccccacacaaacgtccggctcttctcaggccaggtggctgccctgacatggcatgatgtccagcgcatggacgtcacccttgcactggagcccagccaaccgctgcatgacagccctcttcccccttgaaccccaagtctgcacacaccagtgcctcctcttcctggatcctcctgatggcctgtacacagaactggggtgtgggtggtgggtttttaaatgaaaaccagtccacacatgcttgagggctggagccgcctgccgtcagggcagtgccatgctgacctgctgaccgttccattttgtcaaggctcatgagcaattttaaatgtttactcgtgggtgaaggtttttgtttcagctgtaccccctatagcagggccatggggagcctaagaggttctccttggcacctgtgcgcgctctgctgttgccggtgagtctgaaattccagggaatgccgtgaggggccctctggcgtttgttcttggacaaatgtgctttcacccccattaagccctgcttgttggtcttcatttcatttgtgctgcgctgcccctcctgagggacagacgctcaggcagtgcttccttccagatgggctatctgaagaggaacggggacgggagcctgctctacagcgtggtcaacacggccgagccggacgctgatggtgggtgcccgctcagggctggctttatgctctccgtacactgctttttgggcctccgttgatattctgtaatgaaaagctgagccatggccatggggcagcatgagaggctgcccggcttcccgaccttgctcgcaccccaacgtgacacgtgcgctgagtggaggtgtggacggccatctcgcggggcgggggtgctggtcagacctcggtcagggctccttgggagcagcgggactcgagccatgttcctgaaaagctgccgagaggcctttaaggccaaacctcactagcatcaggaagggttcacatcccgagctcagtcttcagcaaagaccagagtgctggcccaccctgctccttgaatctggaaagcagtcttagaagagctgttagatgatgacgtcaggacagtgttgaaggatggtgcacactaggcctgtccagcccgtcaggggcactttgttggcctggaagtaattaagaggcttgtcctttgaacgaaagtgtgatcacttgggtttgaaacgcactcgcgggctcgcgtgaggaacgtgcagcctggcacgggttgcctctagattcctgcttccctcgctctcaccctcatgcctgctggtggtctttcacccagctccaccatgccagcgtggttttctccagtgtgtgaaggatagaaacgggctgatctatataatcttcagcttattttcctaaagaggatggaccgttgatccatatttagcttatgagagtgttgagaccgaggcagcggttgggaactgaggaactcctgctctgatcccatgagtggtgtgtgggtggtgacttgagcatggcagacatgctgtcagatgcacgcgctccaggtacacgccaccgtttcgctgttagaacacgcaggacaagatttcttcttggagttgttgagtgatccatttgaaaatgttctcaaagtattgctggtgtggtggccaaagatgtggtattttttattgtgttgactttggggtgataaagaaatgaaattgaaatgcagttagttttgagaatggtagttggcttttgcaaatttgttttcaggtgatcattataaaatccccccacctgcaccaatcacatccactgtgatgtggagaaagggctcatggcactgcactggggcaccgcgttggggtcacacctgccactcacggcccgccatgtccgtgttgcagaggaggagacccacccggtggacttgagctcgctctccagtaagctactcccaggcttcaccacgctgggcttcaaagacgagagaagaaacaaaggtaaatccatagtcccgtcccaggagccagccggagcagggaaggtggcaggggtttagtggagtgatggcaaatgctgctgattcgtacttcagagtttcctcactgtacctggattcatgttttcacatgggtaagattgagatcagttacagtgacaggaatgaaactcgtgttggatggggacacacacagcgggcaccttggttgcatcactggacttcaccttggtcagtgttttggggtccttgtgtttctgtccctgtcgcattacagaacatcccttcaggggtcagaatgtgtggcccctgggccttgggtctggcctgtgtgttctctccatctgcactgctgcctcctggctgcgctgctgctgggacctcctgcatggccccaccctccacttctccacctctgctgggtccctcccatgcagacagctgcatggacactgctcctgccggccctttctgcctcagccactcacaccatctgctaatgggacagctcactcttccctccaaaccatggccttggctcaagagcttccttgtttctggaatgttctttcctccagctccaggtgttgaaattctgcctggtctgggtctcctgttgaaggacgccctccactgggaaggatcctcttgccttcaccacttgtcttcaccagcccctgctcccctcttcctcttggggctgttgtggttgttgatactttttttttgttgtgtttgacacacatcttcttctcaccctctaacacagttctcaaccacagcacttttgtccctggagatgttggcagtgtccagaggcgtgttgatggtcccactggggttgggggtgctgctggcaccagatggtagggagatgccaggggtgctgctccacaccctatgggacactgcacagtacacctggcctgtgtcccccacagcgagagctggccctgggcaggcgtggtccctgcggtgtgtgttggttgggatcctccacagtgacagacggtgcgctctgcccacgtttccacacagctcttttgcttgtggagctcacccctttgcagagagctcatttccctgcggtctttggcctgcagaagtaaaatgaggggtggtgaattacacccctgctggttacacatggaaaactcaggagtgagaattttgtggagagcaagagaggtgagactggggtgctggctgccagccaggcggtccctcagcccctggagaagcggggtggggcctgcacaccgagtccttccagtgagtccagtgatgctctctcctcttcctcccagtcacctttctctccagtgccactactgcgctttcgatgcagaataattcagtatttggcgacttgaagtcggacgagatggagctgctctactcagcctacggagatgagacaggcgtgcagtgtgcgctgaggcaagtgtcggccccggggagcccttggctgctgtgcagcttgagctgtgccctggtgtgggacgtggagtcccacatgtgtgcgggtggggagagagccatcttgcagccgtccatatggcagcctgggtcttcccgttgctttcagtcatccacacaggtctgccacaggccagtcctgaaatctgggtaactcagtcagaaggtggtgatcagggcgtagcttgtgaaggggttgggggtgtgttgagcacactggatcgtattgaattgtgtttcctaagtgaccgtgctagctcaggctgcagtgacaaagcaccacagactggggtcttattcctgggggctggcatttgccatgaaggtgtgggcagggctggttcctcccgagccctctctcctgggcctgcaggtgccgtctttccgtcttttccctgtgccttcccggggccatcctgcgtgtgtctgtgtcctcggctctcctggagaccctaccgttctcaaggaccctggtcatattggatcagggccaccctaatggcctcatgtcactgcagttacctctaaaggccgttaaagtccccagctccaagtataatcacattccaaggcactggggtcgggatttcaacttacgaactctgagcgggagatacaattcaggccatggcaggctaaagtataatactaaaatcacaaatgtagcagcctgcttttccgtagaatgttgctgtgtgtattttttgttttctgtggcaaaataaaagtgggtcactcttgtaaaatttggcagtttttaatggagaaagcaacttaattttgctagcctttaagaaatggcatcttgacatatcaggtcttcattttattccaacaagaatgcagaatatgttttaaaaataaacaagaatccccttagtgcttcataaaataagtgaaaacctgaccctgatttttgtagcaaaaaatgaagtaaacgtggaaaatgaagaggaaattctagtaggagacgatggggtcataagtcagaaattaaagagttgtttgctccccaccctcccgccagccctcacctcacccctgctgtggggcctctgcccagcctgggccaaggccgtggctcccctggtgccatcctgtccgcagtcggctccctgtccacggcgtgtggctccttcaggacatcagtcacctgttgtccatgtggcacatcagtggactgtgcacaccccccgccttccccagctgtctgtccctagaacgcatcaggttccatgttgccgtgaagaaagatcattctgaccccgcctgccttgtggctgcaggggcaacactcagaagcacatctcctagggcctggcatgaggctacgtggtcccatgccagcctcagcgcacagacacccagatcctctgcccctgagtgctgcttccgtgcacgctgcgagtgccgagcttggcctgtcccgggctgtgggtgttttaacaaagacacggagtctccctgttgcctcctgtgaccgtggttcagcttctgaccagcagccatccttgttcctctctgtgaaggtctttggagtctggggagcccttctgaggcgcttgcttggtgctttgattctaagactatgcatttttgttcctggcctactttttagattgtattatattttattttttaagctgtttcatgtatatggccaagaatcaggatggtactaaaaggagtgccttggaagtgtcggcccacccctgccccacgcgcccattcctcaccggcaggcactgcccactggtgtgcttctgtccacacccacttgtgtttttaaacgcataacgagtggaaagataggagtattcttttacagatgataacatagcacacgtgtgtttctaggctgtgtgcatgtgtattcatgcttttctccctctccccactgagatctttttaacctttttgggtcatggaccactttgaaaatctaatgaaaactatcagccactccccacaaaatacccgctctgggagggtcacggggaccgaggagctgcgctggcacggcaggcttctgtgatcagggctgccgcgagggccaggcgacaagggctcctttcagcgtaccttctcccgcggcccctctagcctgcaggagtttgtgaaggatgctgggagctacagcaagaaagtggtggacgacctcctggaccagatcacaggcggagaccactctaggacgctcttccagctgaagcaggtgggctgcactcgcaccggggttggggggcaggtgcccttggggtgctgctggcgggaagagcctgcagcctgaccaggctttcggggacgaggagctgtcgcagggactctgctgccggctcctcgtcaggatctgcgtgcttctctgcacgggcaggtgtcccccggagaccgtgactgtgtgctcaccactcccagcgcactcgtaactctctatttccatcttcatctttcatctctgtcactttccttcatcctgaagagtttaaaaaaatatttcctgtagttcagatttactgacagtgatctctcagcattggcttctctgaaagtctttatttcatcttcatttttgaaattcattttccctgtgtatggaattttagatgaccttttttcccatctactgtaaaataggtttgacttgcattgttgctataaaaaagcatctgggccaagcatggtggctcacacttgtaatcccagcactttgggaggccgaggcgggcagatccaaggtcaggaggtcgagaccatcctggctaacacggtgaaaccccgtctctactaaaaaatagaaaaaattagctgggcgtggtggcaggtgcttgtagtcccagctactcgggaggctgaggcaggagaatggcatgaacccgggaggcagagcttgcagtgagccgagatcatgccactgcactccagcttgggccacagagccagaccccatctccaaaaaaaaaaaaaaagcgtctgaagtcattgtcatctttgtttcccacatgtgacgtgtcttttcggccagatctgacctgaagtctgcttttgtacagaccacaagccaggaatgtcttttacatctttaaaagggtgtttacagaaaagaaaaggaagaagaagatgcttcgggcagcgtgtggctcacagagcctcggctgctcactcccgggccctcggcaggacgggatctggccggtgctctaggagcttttgaacttttatctttctttatcagtgattttcagcattgtattttccaggtgccttggtgggcttttctttgtgttctacttagcatttgttgtttcttggatctgtctgtagttttcatcatatttggaagatttttagccattactcaaatgtcattttccaccactgcctcttcctcctcctgctggactgtggcactctgaggacagctcgtgttatctcacatggcaccgacactgtgctgccgcaccaggctgctgcagattagtttctgatgctgttggctaagttcactcacctgttctctggccacacctgaacctgttatcccagatcagttttcactgatgatgtcgtcccgcgctgctcatcacttgggctccatttgctgcttttgtgtatcttctctcatgttgtgtgtatgtttttccttacatccttgagccacgttcgtatttattgctgctgcttttgaagtcctcagtcactgagtctgtcgcctctgccgtttccggttctgtttctgtttgacactttgctgcacttggagctgtgggccattgtgcacgtgcctggtggacagtcctgaccctcgctgtctgataaaaaagcactggccacatggccgctgggaaatttgaatgtggccagtctgaatttagatgtgatgtgtgagctacaaactagatcttaaaaaactaatgcaaaaaacgtaaaaatatgagtaacgatttcttatggcccacatgttggaataaaaacatcttcaataaatttgtctaagtaaaatgcattagttaattgaatgttaagagtaattttaatgtggcagctagaattttaagagctgggtatggtggcatgcacctgtcatcagctccacaggaggctaaggtgggaggatcacttgaggccgggaggtcgaggctgcagtgcgattgcaccaccgcactctaggcaatagagcaagaccctgtctcaaaaaaaagataaaaaaacatttagaacgaggcgtgtgctcgcgttatgtctgctgggcaaggctgatctgggttctttctctctttcagagaatactgatgatcattttggcagttgttacttgtggataatggccggatcctaccaggcttgtctggggtgggtccagggcagccttattctagggttcgtgtagccttaatgctaaggagtggcccttcggggagccctgtcttttcagcgtggtctctgctgtgggtggagttcggatgagtctctgtccttcccaggctctgagaacgtttggctcatggctcccaaggaggggtcagtcccttgacctgacggtttcatctgtgcttgttacagatgaactggggtccacatgcctggtgcagtaaacccagacacccatgccgaggttttgtcatagaaaggacgtgtgtatttgcagggcgccaagcaaggtgggccaggaggctcatgtataaatcctggcctcccccactggctacaggcaaggcttgtaaagcgggggtagatctcaggaaagcagaactgaaaggcagagtcgtgcataaggacacagaggttacatgtcggcttggcctagaagggcggtatctgcaagcagggccttataggttataggtggattcaaaggttttctgatttgcagctggttaaggaggagaagctttgtttaaaaatttggggtgaagagaaaagaatgttagcttaggcttgtgggtgtggcttcctccaggcccgtcaggaagaaattcagaacaaagaacggggtcagagctcagtcttcagtttctccttatctgagcccttcatgccagtagattagtttgttggggaggtcctaggtggagggtccaggtttctgaaagacaactcagggatatacattaagatgttatctttactttctttctttttttttctttttgagacatagagtctcactctgtcacccaggctggagtgcagtggcgtgattgtggctcactgcaacctctgcctccctgattctagcgattctcctgcctcagcctcccgagtagctgggattacaggcatgcgccaccacacctggctaatttttgtatttttagtagaggcggagtttcgccatgttggccaggctggtctttaactcctgacctcaaatgatgcatccatctcagcctcccagagtggtaggatgttgtcttttctttctacagggaacagtgctgggaggttgtctttactttctacagggaacaaacatctgtctccaccttccttgactgttgttttaggatactgttaccttcttgcttatcaagtcgctgatttacttctcagagttatctaagtaactggaatttcccttgaaggaactcaagattttccttgatttccatgcttggagggctcgcaggcccctgaagagagggttgcagccaggcgggagtccccaagcagatttctgcagctctgaggagcagccccctctgcagagcctgccccaccagctcgtggttctgactctgagctctggtcaccctcccgtgtcgtggcctgcaggctccttccaggcaggaagctggcccccccaacacagcgttagaatcaggcattgctgtggggtagggaaagtgaccccctcacagcccggaaatgggaaggctctgtggtttcctctctgggttccagttagattcctttcgggaaagtgaacttggatatggagttgagtgaccccgagtgcacccccgcagtaaatgatgctcaggactaaagtgactgaaggcttttggggaaacagatgcgaacaaaacagcagtattcattttctaattaaaagcttgtgaaaatcgatgtcattgaatctgagaaacattatttcgaacaaagatcacacatgatagagtgcatatactacagcatcgctccaccggagcacaggacgggtgtgccacggaggccgacagttgtggaaggagacaatgcacggcccggcccacctgctgcaggcaccgccaggaggtgtctgcctccttcgctcctgcgcccacggcaggcccacctgccacaggcactgccaggaggtgtctgcctcctttgctcctgcacacacctgcccctgcaggactcgagctggtctcaggcaactggtcatattccaggtggaacccacgagacccttcttatcctaagcttcccataggatctaggcacatcttttttcttttgttttaagccacccaaggctgccgaaacagcatcggcgtcctctctctgtccgctccatctcgccctgtggagcgctgtccccggcacacaatcctggcgctgaggagggggctgggcgtggttgggtcagtgactggtgatgaagaaggtttggaggacccccagccttaatccattaagatggtgtgactggggagcagaattcgcagaagcgcacaagccatttttacgtgtgtgtaatgtcagcgaattgtccgtttctatgaaatcactctggaaaaactagtttctgtcctcatttacttttcagagaagaaatgttcccatgaagcctccagatgaagccaaggtaagtttttgaacatggaagggcaagccaaatattattctcacagggaaagttgtatccagcctttgaggtggggaggtctgtgtgtatgacggcgtttagaaatggagtccgtttctgatcagtaagatcaatagtgtttctcctcctcttttcatgacttgaggtgagtggtgtgaagccgcagtacattcttccaaatccagaaatgggtagtttcagagcagttggccccgctgggtgctgtccttgtgggacacccttgcaagggttgggcaggatgggccttctcaggacctgcttggtgagtagcaggtgttgtcccaagcacagtgggctctggggccagtgctggcagggcagtgctgtgagcccagactgctgcacctgccgattggcaggtgtgtgtctctgacagtcagcaggcatgggcttgggctttggtccagggtccctgggaacctggagagaccccttaacatctgtttgagggcagaggccgagctgcagggagctgatgagagagtatgtgttagggtggctgtagtctagccctgaggacacgccttgctgggaaaggaaagggaacgttgtgtgctctgtgaagtgcctgagaaatagtcctggaccctaaaattctgtgtgtcttcctgttgcataatgtccctccccaatttctttagccccaaaaagttaacaaaggattgttagtttgcgttgttggtctcagtaggtttttctttgacatggatttgcatatccattcatgagctccaacctgacagtcccttgaacagcagcctcagtgaacagggcttggtcttgtcacttactgaggggtgggttcacgtttagcaaataattagagtgatttcaatagcgagtgtgaagtaacatttcgtttttcgtttgatgtttgaattgctcttgatgtgtgtctgtctccacaggttggggacaccctaggagacagcagcagctctgttctggagttcatgtcgatgaagtcctatcccgacgtttctgtggatatctccatgctcagctctctgggtgagttgtaacaggcactttcccacagcacctggtgaaggtgtgatagaggaagaaaacacacggcctccacctcctctcccttcctgactagacagggatgccaggaaaatcttttgttacggtatttggtctttgtcccaggtttttggaccatagctcctaaaatccttggaatctcctgagtgttgtgtcttttttaagctaatgagaagtggttgcagcccctgggtaacctcaggatggggtctggccgatccccagtggctgtggatttcatcagtgttgcctaccgcctccatgaaatcccaaaaggtggggttccctagggagcctcattgctggacgcctggaggctctgggaagaaggcagccccttcccacgggtcccctctgcatcgctcccacctggggcccatccgtaccctgggtgacaccccgcagaataaatgggtaaatgtcagcctagtgtgtggacagaagagtcaaactctgcagggtatttagagaggtttcttctgagtgaaatacgaagaccgtggctgtgacacagtcccaggaggtcccaagaacatttgccgaggaggactggtctgcagcttgttttgatatgttttagggagacagaggacatcagtcagtacatgtcagttgtaacagtggttcgtgttggaaaggcgggacaacttgaagcgaggaatgtcgggtcgttggtggattcagagatttcctgattggcatttggttgaaagggttaagctctgaagagctgaagtcagaaagaaatgtgcatggttaacataagcagttgtagaagccaaggttcttgtcacatagatgaattcagccttcaggtagcaggcttcaaaaagagtagagtagatggcaaacatcactcatgaggccctcaaaggtaccagattgttagttaaatctctgttgggtcaggaagagacttggaaggggcaggagattctctacagaatgtagatttttcccacaagatacagctttgcagggctatttcaaaatatgtcaaagggatatattttgagataaaatactcagtttctttcagggcctgccatctatcacgtgatgctgtactaaagtcggatcggaatttggtgtcttattaccacggagtctgctttgtcaaccttcagatttctgttttaaagttaatgctggtcagctgtgcctgaattccaaaagaggggataatgaggcaagtccagtcccgcttcacatcatggcctgacctcattttccaggttgactttggagtgcccttggccaagagtgggaatccgttcagtccatggggcttagaattttatttttcatttacagatgtttccctgacttctgtgagtcactctaggaaattcatagagctcaagtctggtcttctgggaaccccatttgatagctggttagtcggaagcacagaccacagcctggggctttcagttggcatcagccggtggggccatcctggagactgtgccctcacctgggaggctgccggtgcagcgcccccgagctgttctgacctccagcagcatttgtggtgcctggatccatgggtcccactgctttgcgtggtgacgttccacctgtctctgctggctctgttcaggcacttatggccgtgatttattataggggaagggtgccaacagcagcagagggacaagagtgacatccaaggaaacccagcgtgaggcccagatccactcccagcagaggcacatgggacacgcttaattcccaggagtgagctgtgacaacacacgtgaaatgcccaccagggaagcccgtcagaggcttggtgctcggggtcacagggcacccccttgccaggcaggtaccaaagtccagaccccggagggaaggtgaactttagcataaaccactctgctcctgttgttggagctgtgaacaatccctagcactgggggggaccccagatgccagccgtggctggccttgccttccaggggaccctggccccaggccgctgtgacagcacttttctgcacagcagctgtttttctcagcacttctaagattttgtccttattactacttctcagcagcttggtgctgatgggctttggtgctgatgtgctgtggtgtgttagtctgtcctcacactgttataaagaaccacctgagactggtcattggtgaaggaaagaggtttaattgactcacagttcctcaggctgtttgggaggcatggctgggaggcatcaggaaacttccaatcatggcggaagatgaaggagaaacatgcatgtcttcacatggcggagcaggagagaacgcaaagggggaagtgctttgaaacaaccagatctcatgagaagtcactcactatcatgagaacagagaacagcaagggggaaatccatccccatgatccaatcaccttccaccaggaccctcccctaacattgggaattacaattcaacatgagatttgggcaggaatacagagccaaactgtatcattctgcccctgactcctctcgaatcttttgtccttttcacatttcaaaacacaatcatgccttcctaacagtcccctaaagtcttaactcattccagcattaactcagaagtccacagtccaaagtcttatctgagacaaggcaagttacttctgcctgtgagcctgtaaaatccaaaacaagttagttaattccaagatacaaatcgggtacagacattgggtaaatagacctatcccaagagggagaaatcagccaaaacaaaggggcagcaggctacaaatctgaaacccagcagggcagtcattacatcttgaggccccgaaagaatcttctttgattccttgtctcacatccaggctacactgatgcaagggatgggctcccacggccttgggcagccccgcccttgtggctctgcagggtacagcctctgtggctgctttccctggctggtgttgagtgcctgcagcttttccagatggcatggtgcaagctgtcggatctaccattctgaggtctggaggacggtggcccttttctcacagctccactaggcagtgccccagtgggtactctgtgtggggtgagggctccaacctcacatttcccctctgcactgccctagcagaggttctccatgaagggcctgcccctgcagcagacttctgcctggacaccctggcttttccatacatcctctgaaatctaggtggaggctgccaaacctcagctcttgccttctgcctacccacatacccaacaccatgtggaagcttccagggcttggggcttgcaccctctgaagcatcgacccttttagccctgtaccttggccccttttagccatggctgggacacagagcaccatggcacatgtcccagtgcttcacagagtagtggggcccttttccctcctagacccccaggcctgtgatggcagtgtctgggcaaaggtctttgaaatgtccctgaggtattaacatttggctcctctttacttacgcaaatgtctgcagccttcttgaattcctccccagaaaatgggtttttcttttctaccacatggcaggggtacaaattttccaaacttttactctctgcttcccttttaaatataagttccagtttcaggtcatttttctgtttgtgcaagtgagcataggcttttagaagcagccaagtcacctcttgaatgctttagtgcttagaaatttcttccaccaggtgccctaaatcatctctctcaagttcaaagttccacagatttctagagcagggtcacagtgctgtcagtctctcctaaagaatagcaagagtgacctttgcccccattcccaatggggtcctcgtctccatctgagaccacctcagcctggatttcactgtccatatcactatcagcatttgaatcatagccatgcaggaagtctctaggtagttccaaactttccctcatcttcctgtctttaagttctcccagctgttctagcctctgctcattacccagttctacagggcatgtctgtagtcactgttactacactttgtctactgattccctgatatcagctttgagtctctcctagtttggggctgtgttttcctgctccttcgtgtgtgttggaatcatttactggataccaggtggagtttgtagttgatgccgttgggtgataggtctgcttacacacctttgaggcctggcggcctttgttctgagtttgagaagtggcttagactctgtgggaccttccccaggcattgtcagggtaagcccagagtcaccccggggagggtgaagctggtcccctggctggtggggctcaggtgaccacagacctgtgtgcaggttcttggcatgtctggtgcacagttcaggagtgggctgggagctcatcctttccacctctcagctggacgtgctgggtccccgatggctgtggcctggaacctgctccacagtgaactgggcgggtaccagggtctgccggcctcttgcaccctccatccagctgtctggcctttgtgtgttgaggacaggagcccacaccaggcccatcctgggttgtggccacatccctccacggtgtctgtgcgtgccatgacgtggtctgtgcggtgctaggtcttgtctgatgctttgcttgagcaaaggacgctgggtgtctgccttcccttcactcactgctaagggtaatgtctgtcctccaggcctgtccacctgtctgctgtgagctcgtccactcagtttccagagcaattattgtcctgagcagagaggcctgtctggatgggcagtgtgctcctgcccttagggtagacgggccggctgagctcagccgacgtggggtcatgtcctgtttacacagggaaggtgaagaaggagctggaccctgacgacagccatttgaacttggatgagacgacgaagctcctgcaggacctgcacgaagcacaggcggagcgcggcggctctcggccgtcgtccaacctcagctccctgtccaacgcctccgagagggaccagcaccacctgggtgagatgcccccacccacgccgcatccccagagaccccagcacagtcagtgtgacgtctagacgaggtagtgggcagcattgtagttgtgggaaggctggaggctgcatgggcagcggtgctgttgtggaggtcggtttccacagatgtccttgggtcgcggcctcggtgcagagccggagcggagctgggatgctggcctggccgcacgctgggttacaggctttctgccagataccgtccccagttctgtccacccctgtgccctctgtggaccctggttccagtgtttctgctggggagagagcatggcttccttggccatgtgtcctggagtggttttggtgggtggggttttgttcccacttgtaaacactggatcactctgtcggcagagtgaatcttggcagttcttcagctgcagaggtttagctgcaaccatctgcaaaaaggccggtattttcgattccattgaccttgtgatcagggtgttaacagtgcatggagcaggctttggagggccgtaggtcaaagtctgaggcccccgatatcccagtatgactttgggaactgccctgaggtctgggctgtgtctgagggactcctggccaaggagcgttctctccttcagaagcagcatccgtgctgtgtgctgggtcagggcgacccctggctccagggtctctacacacagctctccttggcagggagtgagaggtgcccctgtgtcctgggagtccctgagccctggcggtaggtcaggaagcagaccttgggctccaggagccccacgtgtgctgcgggtctgtcctgtgtgttgaagtcctgtgttctgaaaattgcaggaagcccttctcgcctgagtgtcggggagcagccagacgtcacccacgacccctatgagtttcttcagtctccagagcctgcggcctctgccaagacctaactctagaccaccttcagctcttttattttatttttttagttttattttgcacgtgtagagtttttgtcatcagacaaggactttgatcctgtcccctttggcatgcgggaagcagccgcggggaggtaatgaattgtctgtggtatcatgtcagcagagtctccaagccccacgaaccctgaggagtggagtcatacgcgaaggccatatggccatcgtgtcagcagagagagtctctgtacacagccccgtgaaccctgaggagtggagtcatacacgaagggcgtgtggccatcgtgtcagcagagagagtctctgtacacagccccgtgaaccctgaggagtggagtcatacgcgaagggtgtgtggccaggctgcagagctgcgtgccgtttgtgtccgagcatcacgtgtggctccagcccttgtttctgccagtgtagacacctctgtctgccccactgtcctggggtcgctcttgggaggcacaggcatgggtgtgtctggcctcattctgtatcagtccagtgtgttcctgtcatagtttgtgtctcccaggcaggccatggtaggggcctcgcaggggccattggggagcacagggccaggctggggtgaggagagctcccctgttttctgtttaattgatgagcctgggaaaggagtgtgttctgcctgcccgttacagtggagcgttccgtgtccataaaacgttttctaactgggtgtttactgagtgcccatttatgttagtatttgcaaaataccataaacacagcttccagtcacaagacacagagcgccaaagtgcacccaactgctcccagcacgcgagggtgctgtgtcctaccagctggtggtggtcctctggcccagctgtcgtgcccacagtgacatcaggtgcacctgtgcgcccaccagcacctcctgcagggtgccctgctctgtgaccatggaaacttgaaagtgaagaagccacaccttccagaggccaggcatggatgggagctcagtcgtcctgggggcagcggcatttgtgactctgaagaggccccgggggcagtatgcagtgcctgggccctcacaccacgggaccacagggctgaccctcgcaggccattgacgctgggtgagcagggtggccggtgctctgtgtggcatgggtgagcattctcaggtccttcctgggccctggtggaagtgggagcaaagccattgcctctggacagagcatgaagaagccacagtagctgactcacaggaagagtttgtgaacagggctcagacgaggggcagagcctgggctgcaccctccgtgctggctgggaagcagactccacgcttgtctgcacagctgcacctgccatcctgtccctggatgccaccaagtgcaccgttccagtggctgcctgcaggctctgctccccacccccaactctgggaggacgcccttgctgcagggaggctgggaaaggcagcatctgcagtcccaagctcagtggggctgtgtaccgcacccagcttccactggggctccagaggccaggattcccttaacaggacagacacttcccacagccgggtccccagggtacagccttggagagcttgggctcaggaacaacgtggctcctgacagcaggttgctgtggagtcaggtgtcctcccaccatccgatctctgaacacctcgctccacatccactgtgagtggaggagccctcgccacatgctggggccccgtcttaaactttgcagccccaggaccatgagccaaataagcctcgtgtctttcaggttacccagtctcaggcattcttttacagcagcacaaagcagcctgcatcgagatgacacagctgtgccctcatggtgcggcaggcgtgggcggccgtccctctgggctcgtagcgctctgaagcagtttctgggtggaggtcaggtgcagcccacaggcagtgctgagctgcgggaggcagccaccctgcccggcttcctggtggcctggcccttgttcctgcacccctcactccccgaggtccccgagactggctatccctctgggcggcattgttggtccacagggtcccagcagagaggggcactgggcagcagccaccacgtggcttcagtcttagtgagtgtgcaatgaggggacatcttgaagaggcctatgggtgacctacaggccgagaggctcccctcgggtcaggagatgggagtgggctggagagtccagggctcctcccgctgccccttgggctgcgcagatacggggccaccctggtcagctgatcctgcaggacttagagggccctagtggtggctggagctgtccaggtaggtcatgcgtggcctccccgggattccggcagcctctgctcttgctagcagcccgtgagcccctccaccctcttttgtgggtacaccacctttgggtgtccccaagcccgcatgccagggcacagagcagccacggtggagggagcaggggctggggccttccctgagctcagcttctctctggtccctggatgcttcctttgtggtcacacagtgactgctgctgtggcagccatctcacgttgaggccagaggaggggcagaggggaacaggctgccacgcctgtgtctgacaggacacaagagctttccagggaggccccacagacatggccgtgtctcagcctggggagcggctgggtgtgcagctccagctctccctgtgcaggcagggaacagctaccggtaccccctaccctgctccccgcttgatgtgggcagaacctgggtaggggagggggagatagctgggagcagagggcggcagtgacagggaccacttgcagattgtggggcgtgagggccagcagctgtgcatcgccggccagggccctgcaagcctgtgcttgtctgagccaggaagcaggagggtggtgggacctgctgtgtgggaggtggcatcctctgggcttggcttgggccacacttccaacctctgtccagcccctcccaccctttggtggttctggggcctgcagaatccaagatccgctcaccccgtggtgccctcagggctttgttgcaggtggggaaactgaggcagatgcccagtcgcccagaactaccctaggtcctctgagtgtggctgagcctcccccaccatctgttaaccttgggcacttgtcaacttttaggtgcctgacccctccccgagctcacagcttcctgttctgaattgggaggcagaggaggggcaaaggggtctcctccaagacctcagtctggatagggagtgcgtgcatgggctgcaggtgtgggggtgtgcaggttcggatggggtgcaggtgtgggggtgtgcaggttcggatggggtgcaggtgtgtgtgcaggtgtgtatggggtgcgggtgtgggggtgtgcaggtgtgtatgggggtgaagctatggggtctatgcaggtgcgtatgaaggtgcatgtatgggggtgtgcagatgtgtatgaggtacaggtattgggtgtgtgagtgtgtatggggtgcaggtgggtgtgtgggtgtgcgagtgcatgatgtgtttggtgtgaggggtcggggtgtgggtgtgctgtggtttttgtctccaccaaaatttaagcgtcataaccgaatggccagtgtgatggtgttaggagtggtgaggccgtgaaggggatgaaggtccttgcagaccaggcctcagtcagcagagacctctcaccctcccagcgcctccacgtgagaacacagtgtctgtcccctccgtaggatgcagcagaaaggtaccaccttggaagctgagcgcccccctcgccagacaccagacctgctggctcctgaatcttggacttccagcctccagaatggggagaaataagtttctgttctttaaaattactccgtctcaggtattttgttacagcagcacatggggactgagactgggtgtgcaggtggtggaggagggtgtgcaggtgtgtttgagggtgtttgcctcaatctttagacagcatcagggccacaatacctgcaggtgtcaggacgttaactgcagacattggtgaataaaagccagttgtcctgagtttggcaagaaaacctttggaagaggcctcacaagccactccaggctacaagacggggcgtttgtcctgggggacctgccgtcacccttagtggtccagcagcaccgtctcagcctctctggccagagcagctgccccgcaggggcccccgagtcctggagagctctgtcaggtaggcttccctgccccttccccccatgtcgtcctgggcacccgccccagcccagctgttcctcttacttgtgtacctcgtgtgtgcccctgacagacaagatgtccatgggcagcagacctctggccactgcaggctccaggcaccagagtgagacccagcaggcttcagccggcaagagggcccctgactgtggacgactccctcacggtggtggtcttggagggacccctgctaacagaaagggtgcatccctgtccagcttcccgtcctcacccccaaagtcagcgtcagcagctggcccgcatcggaggcattcagataggagtccttggggaccccccccaaccgacacgaactggaaatgccttgagtactgagggaggagggctgcagggacaggagggaggatgtccttaaacacacttgctggcccttctcaggagaaacccttccttgtcatgggtcgtggtgaagctgtgctccgagttaagccagtgaccgaattcctgagctcagaggatggccagtgaaacaacttgctaaaatgctgagactccctgcactcgcgagactacgaaaacacgtgctgaagtcagtgagagccacggtggccaaagtctttgctcagaaccagctgcgcaggtccctcccccggttcctcctaggctgagtactctggggtcgcagtcttcctggacgtcgcctgttggttgttgtaggggttttaggtgtttttttttagggttgtcttgctgcattttgttgcggaccacaacgcgttgcaatcctagtcagctcaggggcttttcaggtatttgacttaagagctgagtagctgggcaggctgataagaacagacaaagcgagctcttttgcaggctagtaaactttcatcttagactactttggttcaggtgagggcggctaagcaggtgggaaggcagggagggggaggccgacaagcaggcattggggcccatccaagcaggggcctagtacgtcctgtttttttgtagtttgctgacctaaaccggatcaagccaccttgtcttggaaatggaccactgcatgcattatttcacctccacccgagtccatctcacgctttcctccctcctcatgcccccttgtcccctcatcctcccaactgccatggctttgtgaggcgccctgggttccatggctaccgtgtcgaccagggcagcccacccacttctcactttggtctggagttagcgagagcgacgctgcttttctcttgctcttcagcctggacctgtgatgtgataggcgtgagaaggaactgtgggttctggcggtaaggaaagtggggccttactttcccatgttcccaaaaggcagatgcaatccctgcactcctgaaggagtcaagcagtaaaggcaacacagcaagactgtccgggactcaggctgtgaggatttgtcttatgaggggcctcagaaaaggccgtttccactgtggggagtagaggaatggaaggtttcaccagggtggggtggcttccagagggtccccatggaaaaagcgtggctgagcaggcccagagggggacgaggacacgtttggggggctggaggagagtgaggatatgctcaggggtgccagagtgtcataagggcagggacggtgaggacagactcagggaccacagtgtcataaaggaaggacagggatgaagaggacagacctgtgggaccatggtgtcataaagacaggatggggacttggaccaggatagattcagggaccacggtgtcataaagacaggacggggacctggacagacttgggggaccacggtgtcataaagacaggatggggaccaggacagactctaggggcctggtgtcataaagacagggttgggacctggacagactctaggggcctggtgtcataaagacaggatggggaccaggacagactctggggatctggtgtcataaagacagggatgggaccaggacagactctgggggcctggtgtcataaagacaggcctgggacaggaccgggacagacccgggggaccacggtgtcataaagataggacggggaccaggacagactctaggggcctggtgtcataaagacatgacagggacctggacagactcaggggaccacggtgtcataaagacaggcctgggacaggaccgggacagacccgggggaccacggtgtcataaagataggacggggaccaggacagactctaggggcctggtgtcataaagacaggacagggacctggacagactcaggggaccacggtgtcataaagacaggacggggaccaggacagactctgggggcctggtgtcataaagacaggatggggacctggacagactcaggggaccacggtgtcataaagacaggatggggaccgggacagactcgggggaccacggtgtcataaagacaggacggggaccgagacagactcaggggaccatggtgtcataaagacagggatgggacctggacagactctaggggcctggtgtcataaagacagggatgggaccgggacagactctgggggcctggtgtcgtaaagacaggatggggaccaggacagactcgggggaccacggagtcataaagataggacggggaccaggacagactctaggggcctggtgtcataaagacagggatgggaccgggacagactctgggggcctggtgtcatatacacagatggaaacaggaaagactgaggaggtcatggcatcttaaagacctgatagggtgggatgggcacctgtggtctctggggtctgtggcaggggctggttctcatgtgtggtggtgcagagaaggatggagtcccagtctgagaagggggctgtctccacatccacagatgggggcagacaggaggcgcctggggtccaccacacaggattgcgctgcaggccccggtgctgggcagccagttccccgggcatcggtccggttgtcataacacaggggggcccatccagccttagtcgtgctaaggggacctgaacccaacattacaagtcttcagtgtcatgactcacagatgctcctttcacttgaattgtgtgtgggagtgtgtgtgcattgtgtggtgtgtgtgggagtgtgagcattgtgtgttgtgtgtggtattctgtgggggttgtatgtgtgcggtatgtggtgtgtgtggtttgtgtgctgtgtgtggtatgcggtgtgtagtgcaaatgggtgtgctcggtggtgtgtggtttggtgtgtgtgtggtgtgtggtttggtgtatgtgtggtattttgggggtgtggtatgtgtgcagtatgtggtgtgatgtttggtgtgtgtggtgtgcagtgtgtagtgcaagtgtgtgtgctctgtgtgtggtttgtgtggtgtgctgtgtgtggtgtgtattctgtgtgcgtgatgtgtattgtgtgtattgtgtggtccctgtgtggtatggggagagtgatgcgtttgtggttggtgtctggtacttgtcacgtggtgcacgtgcagtgtgtggtgtgtttggtgtgtgtgttttgcttctgttgcatatgtgtggcgtgaggtgtgtgcgtgtggtttgtgtggtgtgtggtgcatgtgtggtgtgcatggagttgtgtggtgtgtgatctgttttgtgtattgtgtggtgtgtgggtggtgtgtgttgtggattgtgtggtctcgtgtgtttcgtgtggtgtggtgtgtaatctgtatgatgtgtgttgtgtggtttctgttttgtggtttgtgttgtgtgtctgtggtatgtgttgtgggtggtctgtgttgtatgatatatgtggtatgtattgtgtggtgtgtggcgtgtggtgtatggtgtgtgtggtgtcttgtgtgtgtgttgtgttttgtgtgttgtggtttctgtttggtgtgtgttgtgtgttttatgtggtatgcggtgtgtggtcagtgtggtctgtgttgtgtggtgtgtggtgtgtgtggtacgtggtctgtgtggtatatggtgtgtgatgtggattgtgtggtgtggtatatgaacgtgcttacaggccacggtggtatcgaggctgagcgtgggcctgaggggaaggagctgtcctggagtcttctctggctgatgtgacgtccgtgtcccctgctcgctgtcctccatggggtccatgccccttgcttgctgtcctccgcggggtctgtgcccctcgctcactgtctgtcctctgtggggtctgtccccccggctcgctgtcctcggtggggtctatccccactgctcactgtcctctgtggggtctgtgcccctcgctcgctgtctgtcctctgtggggtctgtgccccccggctcactgtcctctgtggggtctgtccccactgctcactgtcctctgtggtgtctgtaaccctcgctcgctgtcctctgtggggtctgtccccgctgctcactgtcctctgtggggtctgtccccccggctcactgtcctccgtggggtctgtccccccggctggctgtcctctgtggggtctgtgccccccagctcactgtcctcggtggggtctgtccccgctgctcactgtcctctgtgcggtctgtccccccggcttgctgtcctccatggggtctgtcttgccggctcgctgtcctctgtggggtctgtgaccccagcttgctgtcctctgtggggtctgtccccgctgctcactgtcctctgtggggtctgtgccccctggctcgctgtcctctatggggtctgtgccccctggctcgctgtcctctgtggggtctgtctccccggctctctgtcctctgtggggtctgtgccccctgactcgctgtcctctgtggggtctgtgccccctggctcgctgtcctctgtgggatctgtctccctggctcgctgtcctccatggtgtctctccccccggctcgctgtcctctgtggggtctgtccccgctgctcactgttctccatggggtctgtctccccggctctctgttctctgtagggtctgtgtcccccggctcgctgtcctccatggggtctgtccccgcggctcgctgtcctccgtggggtctgtccccgcggctcgctgtcctctgtggggtctgtccccctggcttgctgtcctctgtgggttctgtccccctggctcactgtcctctgtggggtctgtccccctggctcgctgtcctccgtggggtctgtgccccccggctcactgtccttggtggggtctatccccactgctcactgtcctctgggcagtctgaccccctggcttgctatcctccatggggtctgtccccccggctcgctgtcctctgtgtggtctgtgccccccggctcgctgtcctctgtggggtctgtccccgctcctcactgtcctccgtggggtctgtgccctccggctcgctgtcctcggtggggtctgtcccggctgctcactgtcctctgtgcggtctgtccccccggcttgctgtcctccatgggatctgtgcccccagctcactgtcctctgtggggtctgtgaccctggctcgctgtcctctgtggggtctgtgccactcgctcgctgtcctctgtgggatctgtgacccctggctcactgttctctgtgggatctgtctccccggctcgctgtcctccatggtgtctctccccccggctcgctgtcctctgtggggtctgtcccagctgctcactgtcctccgtggggtctgtctccccggctctctgtcctctgtagggtctgtgccccccggctcactgtcctccgtggggtctgtctccctggctctctgtcctacatgggttctgtccccacggctcgctgtcctctgtgggttctgtccccctggcttgctgtcctccgtggggtctgtggcccctggctcactgtccttggtggggtctatccacactgctcaatgtcctctgggcagtctgaccccctggcttgctatcctccatggggtctgtccccccggctcgctgtcctctgtgtggtctgtgccccccggctcgctgtcctctgtggggtctgtccccgctcctcactgtcctccgtggggtctgtgccccccggctcgctgtcctcggtggggtctgtcccggctgctcactgtcctctgtgcggtctgtccccctggcttgctgtcctccatgggatctgtgcccccagctcactgtcctctgtggggtctgtccccctggcttgctgtcctctgtggggtctgtccccctggcttgctgtcctctgtggggtctgtccccctggcttgctgtcctctgtggggtctgtccccgctgctcactgtcctctgtggggtctgtgcccccagctggctgtcctcggtggggtctgtccccgctgctcactgtcctctgtgcggtctgtccccccggcttgctgtcctccatgggatctgtctccccagctcactgtcctctgtggggtctgtctccctggctcactgtcctacatggggtctgtccccgtggctcgctgtcctctgtggggtctgtccccctggcttgctgtcctctgtggggtctgtccccgtggcttgctgtccactgtggggtctgtcccccgggcttgctgtcctctgtggggtctgtccccgctgctcactgtcctctgtgggatctgtctccccagctcgctgtcctctgtggggtctgtgctcctcgctcgctgtcctctgtggggtctgtgccccctggctcactgtcctctgtggggtctgtccccgctgctcactgtcctctgtggggtctgtccccctggctcgctgtcctccgtggggtctgtacccccgactcgctgtcctctgcggggtctgtgcccgactcgctgtcctcggtggggtctatccctgctgctcactgtcctctgtggggtctgtgcccgctgctcactgtcctccgtggggtccgtgccccccggctcgctgtcctctgtggagtctctgccccctggctcgctgtcctctgtggggtctgtctccctggctcgctgtcctccgtggggtctgtccccgctgctaattgtcctccgtggggcctgtgtcccctggctcgctgtcctccatggggtctgtccacctggctcgctgtcctccgtggggtctgtgcccgctgctcgctgtcctccatggggtctgtccccctggctcgctgtcctccgtggggtctttccctgctgcttgctgtcctgcgtggggtctgtcccctcggctcgctgtcctccgcggggtctgtccccgctgctcactgtcctccgtggggtctgtccccgctgctcgctgtcctccgtgtggtctgtacccccggctcgctgtcctccgtggggtctgtccccgctgcttgctgtcctccgtggggtctcttcccccggctcgctgtcctccatggggtctgtcccccggtttgctgtcctctgtggggtctgtccccgctgctcgctgtcctccgtgtggtctgtccccccggctcgctgtcctccgtggggtctgtcccccttgctcactgtcctctgtggagtctgtacctccagcttactgtcctccgtggcgtctgtccccgctgctcattgtcctccgtggggtctgtccccctggctcactgtcctctgtggagtctgtccccgctgctcgttgtcctctgtggggtctgtctccctggcttgctcacattttctctaatttggatgtattactttttccaatcatcctttccagctcttggtgcacagtttcgacctgaagtcttcagtcttctcaaagttcttcagtgttttcttttttctctcacagactctccacctttctgttctgaaatcctgacagatgaacgtgttgtctccaggctcaaggctcaaggccgctgagcgattcccctcttctcctctctaagatctgtggtccttcctggatttgatattccaggttttaccaatgtgacttggcgacatcatttctgtttgttggcactttcatttgggttttgtttttaactgggaattgtgtttttaatgtacagggttgtctttctgtgtttctatttccttccttggtaactttgacttttctttctttcttttttttttttttgagatggagtctccctctgttgcccaggctggaatgcagtggtgcaatcttggctcactgcagcctcgacctcccaggttcaaacgattctcatgcctcagcctcccgagtagctgggattagaggcatgtgccaccatgccctgctaatctttgtatttttagtagagacagggtttcgccgtgttgcccaggctggtctggaactcctgacctcaggtgataggcacgcctcggcttcccacagtgctgggattacaggcgtgagcccccgcgcccggccaaagaaccatggtcctgtttcataactgtgagtctctcgggtctcccagagacattcttgtgtgactcaagccccagggtcctgtggccccgcatccgcctggtgtcacatttagaggaccagggaggagccaccaaaggcagcgcagagccactgtatcatggacgttcacgactgcaagaacgaagaattgttaactatttctcaatgtctctcttctcaaagaaatacatttaatatcaacttcattttctgaaggttttttcccaaagagacaacaaggactttgagcgaggcataacgtggcgaagcttcctaaccctcctctgtagagaccgcccagcccctcaagatcaaccccccaccccgtagcggccgttgagtgtctttcactgccgctctgtgacaccacgtccctcccacccctgccgccctccccacctgactggccaggagcagccgcagcgtgttcagaacctcccagccactgtccacccgggcggagaccccagccccgcgtggctggacgggcttccctgtcctgtggaaggggagacacacaggaccctgtgaagtgagctctcgggtcctgctcctctgggaagctcagagaagcctctcagggaggatctggcaggtttcagtgttgcaccagtgagtaacggctcacgagctcttcaggcattgcctcagtttctcacaggcgcagccctctgctcagtgtggcctctttctgacgtgggttttcttcttgcacctggggagtcctgggtgggcctgtgtattcgtgaacgggttgtgggacctgtcgctgtgtgggggctgtcgagcactccccagaacgtaacaaatcctcaggggaactgatgggcggtcgcgcgggcactgggtcctccacaccctggagagccgttttccgttgccactcggctctggccggggtcacattctgcagcatgtctgttcattcccctgggcggggccctgcaccgactccagcccagcccctgctccctctgcggggaacgtggccccaggcagtgctgggccattggctgtcagtgctggtcctggcggctgcattcccagtccccttggtctctgtgacagtgggcggggccggccctcccaggatctgacggcgcaggtcctccccttctgtgtcctgcagatggacacccgctccgggagccagtgttccgtcaccccagaagccatactcaataatgaaaagctggtcttgccgccccgcatctccagagtgaacggctggtcgttacccctgcactacttccaggtggtgacctgggctgtcttcgtgggcctttcctcggccaccttcgggatcttcattcccttcctgcctcacgcgtggaaatacattgcctacgtggtatccttttcatcgtggcatggtctaagcgggaggggttcctggaggaacttgcgatggacctggctgtggggtctgggccatggctgc

SEQ ID NO: 2 - protein, 597 aa, Homo sapiens
MGKKHKKHKAEWRSSYEDYADKPLEKPLKLVLKVGGSEVTELSGSGHDSSYYDDRSDHERERHKEKKKKKKKKSEKEKHLDDEERRKRKEEKKRKREREHCDTEGEADDFDPGKKVEVEPPPDRPVRACRTQPAENESTPIQQLLEHFLRQLQRKDPHGFFAFPVTDAIAPGYSMIIKHPMDFGTMKDKIVANEYKSVTEFKADFKLMCDNAMTYNRPDTVYYKLAKKILHAGFKMMSKQAALLGNEDTAVEEPVPEVVPVQVETAKKSKKPSREVISCMFEPEGNACSLTDSTAEEHVLALVEHAADEARDRINRFLPGGKMGYLKRNGDGSLLYSVVNTAEPDADEEETHPVDLSSLSSKLLPGFTTLGFKDERRNKVTFLSSATTALSMQNNSVFGDLKSDEMELLYSAYGDETGVQCALSLQEFVKDAGSYSKKVVDDLLDQITGGDHSRTLFQLKQRRNVPMKPPDEAKVGDTLGDSSSSVLEFMSMKSYPDVSVDISMLSSLGKVKKELDPDDSHLNLDETTKLLQDLHEAQAERGGSRPSSNLSSLSNASERDQHHLGSPSRLSVGEQPDVTHDPYEFLQSPEPAASAKT

SEQ ID NO: 3 - DNA, 2665 bp, Homo sapiens
gtttccggcgcggcccagcgagctcggcaacctcggcgcagcgagcgcgggcggccagccagggccagggggcggtggcggccaaggtccgaccgggtgccagctgttcccagcccccgcctcgggcccgccgccggcgccgccatgggcaagaagcacaagaagcacaaggccgagtggcgctcgtcctacgaggattatgccgacaagcccctggagaagcctctaaagctagtcctgaaggtcggaggaagtgaagtgactgaactctcaggatccggccacgactccagttactatgatgacaggtcagaccatgagcgagagaggcacaaagaaaagaaaaagaagaagaagaagaagtccgagaaggagaagcatctggacgatgaggaaagaaggaagcgaaaggaagagaagaagcggaagcgagagagggagcactgtgacacggagggagaggctgacgactttgatcctgggaagaaggtggaggtggagccgcccccagatcggccagtccgagcgtgccggacacagccagccgaaaatgagagcacacctattcagcaactcctggaacacttcctccgccagcttcagagaaaagatccccatggattttttgcttttcctgtcacggatgcaattgctcctggatattcaatgataataaaacatcccatggattttggcaccatgaaagacaaaattgtagctaatgaatacaagtcagttacggaatttaaggcagatttcaagctgatgtgtgataatgcaatgacatacaataggccagataccgtgtactacaagttggcgaagaagatccttcacgcaggctttaagatgatgagcaaacaggcagctcttttgggcaatgaagatacagctgttgaggaacctgtccctgaagttgtaccagtacaagtagaaactgccaagaaatccaaaaagccgagtagagaagttatcagctgcatgtttgagcctgaagggaatgcctgcagcttgacggacagtaccgcagaggagcacgtgctggcgctggtggagcacgcagctgacgaagctcgggacaggatcaaccggttcctcccaggcggcaagatgggctatctgaagaggaacggggacgggagcctgctctacagcgtggtcaacacggccgagccggacgctgatgaggaggagacccacccggtggacttgagctcgctctccagtaagctactcccaggcttcaccacgctgggcttcaaagacgagagaagaaacaaagtcacctttctctccagtgccactactgcgctttcgatgcagaataattcagtatttggcgacttgaagtcggacgagatggagctgctctactcagcctacggagatgagacaggcgtgcagtgtgcgctgagcctgcaggagtttgtgaaggatgctgggagctacagcaagaaagtggtggacgacctcctggaccagatcacaggcggagaccactctaggacgctcttccagctgaagcagagaagaaatgttcccatgaagcctccagatgaagccaaggttggggacaccctaggagacagcagcagctctgttctggagttcatgtcgatgaagtcctatcccgacgtttctgtggatatctccatgctcagctctctggggaaggtgaagaaggagctggaccctgacgacagccatttgaacttggatgagacgacgaagctcctgcaggacctgcacgaagcacaggcggagcgcggcggctctcggccgtcgtccaacctcagctccctgtccaacgcctccgagagggaccagcaccacctgggaagcccttctcgcctgagtgtcggggagcagccagacgtcacccacgacccctatgagtttcttcagtctccagagcctgcggcctctgccaagacctaactctagaccaccttcagctcttttattttatttttttagttttattttgcacgtgtagagtttttgtcatcagacaaggactttgatcctgtcccctttggcatgcgggaagcagccgcggggaggtaatgaattgtctgtggtatcatgtcagcagagtctccaagccccacgaaccctgaggagtggagtcatacgcgaaggccatatggccatcgtgtcagcagagagagtctctgtacacagccccgtgaaccctgaggagtggagtcatacacgaagggcgtgtggccatcgtgtcagcagagagagtctctgtacacagccccgtgaaccctgaggagtggagtcatacgcgaagggtgtgtggccaggctgcagagctgcgtgccgtttgtgtccgagcatcacgtgtggctccagcccttgtttctgccagtgtagacacctctgtctgccccactgtcctggggtcgctcttgggaggcacaggcatgggtgtgtctggcctcattctgtatcagtccagtgtgttcctgtcatagtttgtgtctcccaggcaggccatggtaggggcctcgcaggggccattggggagcacagggccaggctggggtgaggagagctcccctgttttctgtttaattgatgagcctgggaaaggagtgtgttctgcctgcccgttacagtggagcgttccgtgtccataaaacgttttctaactggg

SEQ ID NO: 4 - DNA, 25 bp, Artificial sequence
  Synthetic
taatgaggcaagtccagtcccgctt

SEQ ID NO: 5 - DNA, 25 bp, Artificial sequence
  Synthetic
aaagaggggataatgaggcaagtcc

SEQ ID NO: 6 - DNA, 25 bp, Artificial sequence
  Synthetic
gggataatgaggcaagtccagtccc

SEQ ID NO: 7 - DNA, 1435 bp, Homo sapiens
  misc_feature (1069)..(1228): Poison exon at nucleotide positions 1069-1228
gtgagttgtaacaggcactttcccacagcacctggtgaaggtgtgatagaggaagaaaacacacggcctccacctcctctcccttcctgactagacagggatgccaggaaaatcttttgttacggtatttggtctttgtcccaggtttttggaccatagctcctaaaatccttggaatctcctgagtgttgtgtcttttttaagctaatgagaagtggttgcagcccctgggtaacctcaggatggggtctggccgatccccagtggctgtggatttcatcagtgttgcctaccgcctccatgaaatcccaaaaggtggggttccctagggagcctcattgctggacgcctggaggctctgggaagaaggcagccccttcccacgggtcccctctgcatcgctcccacctggggcccatccgtaccctgggtgacaccccgcagaataaatgggtaaatgtcagcctagtgtgtggacagaagagtcaaactctgcagggtatttagagaggtttcttctgagtgaaatacgaagaccgtggctgtgacacagtcccaggaggtcccaagaacatttgccgaggaggactggtctgcagcttgttttgatatgttttagggagacagaggacatcagtcagtacatgtcagttgtaacagtggttcgtgttggaaaggcgggacaacttgaagcgaggaatgtcgggtcgttggtggattcagagatttcctgattggcatttggttgaaagggttaagctctgaagagctgaagtcagaaagaaatgtgcatggttaacataagcagttgtagaagccaaggttcttgtcacatagatgaattcagccttcaggtagcaggcttcaaaaagagtagagtagatggcaaacatcactcatgaggccctcaaaggtaccagattgttagttaaatctctgttgggtcaggaagagacttggaaggggcaggagattctctacagaatgtagatttttcccacaagatacagctttgcagggctatttcaaaatatgtcaaagggatatattttgagataaaatactcagtttctttcagggcctgccatctatcacgtgatgctgtactaaagtcggatcggaatttggtgtcttattaccacggagtctgctttgtcaaccttcagatttctgttttaaagttaatgctggtcagctgtgcctgaattccaaaagaggggataatgaggcaagtccagtcccgcttcacatcatggcctgacctcattttccaggttgactttggagtgcccttggccaagagtgggaatccgttcagtccatggggcttagaattttatttttcatttacagatgtttccctgacttctgtgagtcactctaggaaattcatagagctcaagtctggtcttctgggaaccccatttgatagctg

SEQ ID NO: 8 - DNA, 61 bp, Homo sapiens
tttcaaaatatgtcaaagggatatattttgagataaaatactcagtttctttcagggcctg

SEQ ID NO: 9 - DNA, 62 bp, Homo sapiens
caaagggatatattttgagataaaatactcagtttctttcagggcctgccatctatcacgtg

SEQ ID NO: 10 - DNA, 63 bp, Homo sapiens
gggatatattttgagataaaatactcagtttctttcagggcctgccatctatcacgtgatgct

SEQ ID NO: 11 - DNA, 62 bp, Homo sapiens
tgcctgaattccaaaagaggggataatgaggcaagtccagtcccgcttcacatcatggcctg

SEQ ID NO: 12 - DNA, 287 bp, Homo sapiens
tcaaaatatgtcaaagggatatattttgagataaaatactcagtttctttcagggcctgccatctatcacgtgatgctgtactaaagtcggatcggaatttggtgtcttattaccacggagtctgctttgtcaaccttcagatttctgttttaaagttaatgctggtcagctgtgcctgaattccaaaagaggggataatgaggcaagtccagtcccgcttcacatcatggcctgacctcattttccaggttgactttggagtgcccttggccaagagtgggaatcc

SEQ ID NO: 13 - DNA, 20 bp, Artificial sequence
  Synthetic
cgagtggcgctcgtcctacg

SEQ ID NO: 14 - DNA, 27 bp, Artificial sequence
  Synthetic
tacccatacgatgttccagattacgct

SEQ ID NO: 15 - DNA, 24 bp, Artificial sequence
  Synthetic
cacccgagtggcgctcgtcctacg

SEQ ID NO: 16 - DNA, 24 bp, Artificial sequence
  Synthetic
aaaccgtaggacgagcgccactcg

SEQ ID NO: 17 - DNA, 197 bp, Artificial sequence
  Synthetic
ccagggggcggtggcggccaaggtccgaccgggtgccagctgttcccagcccccgcctcgagcccgccgccggcgccgccatgtacccatacgatgttccagattacgctggcaagaagcacaagaagcacaaggccgagtggcgctcgtcctacgaaggtgaggcggcggcgctttgtgacgcgcggcgggcgggg

SEQ ID NO: 18 - DNA, 19 bp, Artificial sequence
  Synthetic
agcgagctcggcaacctcg

SEQ ID NO: 19 - DNA, 22 bp, Artificial sequence
  Synthetic
cttcaggactagctttagaggc

SEQ ID NO: 20 - DNA, 20 bp, Artificial sequence
  Synthetic
tgcagcctcgaacccagaac

SEQ ID NO: 21 - DNA, 23 bp, Artificial sequence
  Synthetic
tccaatcaaagatcttcttccaa

SEQ ID NO: 22 - DNA, 20 bp, Artificial sequence
  Synthetic
gagcaggtttctgcaacgat

SEQ ID NO: 23 - DNA, 23 bp, Artificial sequence
  Synthetic
gcaatgacatacaataggccaga

SEQ ID NO: 24 - DNA, 21 bp, Artificial sequence
  Synthetic
gagctgcctgtttgctcatca

SEQ ID NO: 25 - DNA, 21 bp, Artificial sequence
  Synthetic
ttggagatggaagtctgctct

SEQ ID NO: 26 - DNA, 22 bp, Artificial sequence
  Synthetic
gcaacttgctagacagtgaact

SEQ ID NO: 27 - DNA, 21 bp, Artificial sequence
  Synthetic
agctctgttctggagttcatg

SEQ ID NO: 28 - DNA, 24 bp, Artificial sequence
  Synthetic
ctgaagaaactcataggggtcgtg

SEQ ID NO: 29 - DNA, 21 bp, Artificial sequence
  Synthetic
ggccctgttctggacttcatg

SEQ ID NO: 30 - DNA, 24 bp, Artificial sequence
  Synthetic
ctgaaggaattcataagggtcgtg

SEQ ID NO: 31 - DNA, 20 bp, Artificial sequence
  Synthetic
cagcagctctgttctggagt

SEQ ID NO: 32 - DNA, 22 bp, Artificial sequence
  Synthetic
cctgaaagaaaccagagagctg

SEQ ID NO: 33 - DNA, 20 bp, Artificial sequence
  Synthetic
cagcagctctgttctggagt

SEQ ID NO: 34 - DNA, 20 bp, Artificial sequence
  Synthetic
tcaccttccccagagagctg

SEQ ID NO: 35 - DNA, 18 bp, Artificial sequence
  Synthetic
gcctgcagaacggagagg

SEQ ID NO: 36 - DNA, 28 bp, Artificial sequence
  Synthetic
accactagcatttcatagggatagatct

SEQ ID NO: 37 - DNA, 25 bp, Artificial sequence
  Synthetic
gcctgcagatctatccctatgaaat

SEQ ID NO: 38 - DNA, 19 bp, Artificial sequence
  Synthetic
ctcaagccgcatccgatcc

SEQ ID NO: 39 - DNA, 20 bp, Artificial sequence
  Synthetic
gttggggacaccctaggaga

SEQ ID NO: 40 - DNA, 20 bp, Artificial sequence
  Synthetic
cttcaccttccccagagagc

SEQ ID NO: 41 - DNA, 21 bp, Artificial sequence
  Synthetic
ccctgaaagaaaccagagagc

SEQ ID NO: 42 - DNA, 20 bp, Artificial sequence
  Synthetic
ctaccacatccaaggaagca

SEQ ID NO: 43 - DNA, 21 bp, Artificial sequence
  Synthetic
tttttcgtcactacctccccg

SEQ ID NO: 44 - DNA, 21 bp, Artificial sequence
  Synthetic
ccaacagccctatggctatgc

SEQ ID NO: 45 - DNA, 19 bp, Artificial sequence
  Synthetic
ctgggcactcactctctgc

SEQ ID NO: 46 - DNA, 22 bp, Artificial sequence
  Synthetic
gtcctccactcttttacagacg

SEQ ID NO: 47 - DNA, 21 bp, Artificial sequence
  Synthetic
attcggttgtgaccaatgggt

SEQ ID NO: 48 - DNA, 19 bp, Artificial sequence
  Synthetic
gagcttccttcccgtgctt

SEQ ID NO: 49 - DNA, 23 bp, Artificial sequence
  Synthetic
tgcctgttccaggttttagttac

SEQ ID NO: 50 - DNA, 23 bp, Artificial sequence
  Synthetic
cccctagcctatatctccctttt

SEQ ID NO: 51 - DNA, 21 bp, Artificial sequence
  Synthetic
taccatcgtggggataatggc

SEQ ID NO: 52 - DNA, 21 bp, Artificial sequence
  Synthetic
ggagcgagatccctccaaaat

SEQ ID NO: 53 - DNA, 23 bp, Artificial sequence
  Synthetic
ggctgttgtcatacttctcatgg

SEQ ID NO: 54 - DNA, 21 bp, Artificial sequence
  Synthetic
aggtcggtgtgaacggatttg

SEQ ID NO: 55 - DNA, 23 bp, Artificial sequence
  Synthetic
tgtagaccatgtagttgaggtca

SEQ ID NO: 56 - DNA, 22 bp, Artificial sequence
  Synthetic
tttattatcattgaatatccag

SEQ ID NO: 57 - DNA, 22 bp, Artificial sequence
  Synthetic
ttttattatcattgaatatcca

SEQ ID NO: 58 - DNA, 22 bp, Artificial sequence
  Synthetic
tttattatcattgaatacccag

SEQ ID NO: 59 - DNA, 22 bp, Artificial sequence
  Synthetic
ttttattatcattgaataccca

SEQ ID NO: 60 - DNA, 22 bp, Artificial sequence
  Synthetic
tttttaatcttatcagatggga

SEQ ID NO: 61 - DNA, 22 bp, Artificial sequence
  Synthetic
tgaacaaacaaaatggcagtca

SEQ ID NO: 62 - DNA, 22 bp, Artificial sequence
  Synthetic
ttctatctacgcattgtatcga

SEQ ID NO: 63 - RNA, 19 nt, Homo sapiens
ugguuuacaugucgacuaa

SEQ ID NO: 64 - DNA, 20 bp, Artificial sequence
  Synthetic
actccagttactatgatgac

SEQ ID NO: 65 - DNA, 20 bp, Artificial sequence
  Synthetic
agagagggagcactgtgaca

SEQ ID NO: 66 - DNA, 20 bp, Artificial sequence
  Synthetic
agataccgtgtactacaagt

SEQ ID NO: 67 - DNA, 20 bp, Artificial sequence
  Synthetic
attaaccggtttctcccggg

SEQ ID NO: 68 - DNA, 20 bp, Artificial sequence
  Synthetic
ggaacactgcgactcagagg

SEQ ID NO: 69 - DNA, 20 bp, Artificial sequence
  Synthetic
acttgctagacagtgaactc

SEQ ID NO: 70 - DNA, 20 bp, Artificial sequence
  Synthetic
acggaggctaagcgtcgcaa

SEQ ID NO: 71 - DNA, 20 bp, Artificial sequence
  Synthetic
aaaatactcagtttctttca

SEQ ID NO: 72 - DNA, 22 bp, Artificial sequence
  Synthetic
tgttgggtcaggaagagacttg

SEQ ID NO: 73 - DNA, 20 bp, Artificial sequence
  Synthetic
ccatggactgaacggattcc

SEQ ID NO: 74 - DNA, 22 bp, Artificial sequence
  Synthetic
tgttgggtcaggaagagacttg

SEQ ID NO: 75 - DNA, 38 bp, Artificial sequence
  Synthetic
ggacccagtaccaggatccgttggggacaccctaggag

SEQ ID NO: 76 - DNA, 40 bp, Artificial sequence
  Synthetic
cttggaggagcgcacaccggtcaggtggtgctggtccctc

SEQ ID NO: 77 - DNA, 22 bp, Artificial sequence
  Synthetic
ggattacaaggatgacgatgac

SEQ ID NO: 78 - DNA, 20 bp, Artificial sequence
  Synthetic
catgaagcctccagatgaag

SEQ ID NO: 79 - DNA, 22 bp, Artificial sequence
  Synthetic
cttcgtcgtctcatccaagttc

SEQ ID NO: 80 - DNA, 43 bp, Artificial sequence
  Synthetic
caaagggatatattttgagattttttactcagtttctttcagg

SEQ ID NO: 81 - DNA, 43 bp, Artificial sequence
  Synthetic
cctgaaagaaactgagtaaaaaatctcaaaatatatccctttg

SEQ ID NO: 82 - DNA, 43 bp, Artificial sequence
  Synthetic
caaagggatatattttgagatatattactcagtttctttcagg

SEQ ID NO: 83 - DNA, 43 bp, Artificial sequence
  Synthetic
cctgaaagaaactgagtaatatatctcaaaatatatccctttg

SEQ ID NO: 84 - DNA, 45 bp, Artificial sequence
  Synthetic
gtcaaagggatatattttgattttttttactcagtttctttcagg

SEQ ID NO: 85 - DNA, 41 bp, Artificial sequence
  Synthetic
gagataaaatactcagttacattcagggcctgccatctatc

SEQ ID NO: 86 - DNA, 41 bp, Artificial sequence
  Synthetic
gatagatggcaggccctgaatgtaactgagtattttatctc

SEQ ID NO: 87 - DNA, 41 bp, Artificial sequence
  Synthetic
gagataaaatactcagttgcgttcagggcctgccatctatc

SEQ ID NO: 88 - DNA, 41 bp, Artificial sequence
  Synthetic
gatagatggcaggccctgaacgcaactgagtattttatctc

SEQ ID NO: 89 - DNA, 41 bp, Artificial sequence
  Synthetic
gagataaaatactcagtttcattcagggcctgccatctatc

SEQ ID NO: 90 - DNA, 41 bp, Artificial sequence
  Synthetic
gatagatggcaggccctgaatgaaactgagtattttatctc

SEQ ID NO: 91 - DNA, 41 bp, Artificial sequence
  Synthetic
gagataaaatactcagtttcgttcagggcctgccatctatc

SEQ ID NO: 92 - DNA, 41 bp, Artificial sequence
  Synthetic
gatagatggcaggccctgaacgaaactgagtattttatctc

SEQ ID NO: 93 - DNA, 41 bp, Artificial sequence
  Synthetic
gagataaaatactcagtttatttcagggcctgccatctatc

SEQ ID NO: 94 - DNA, 41 bp, Artificial sequence
  Synthetic
gatagatggcaggccctgaaataaactgagtattttatctc

SEQ ID NO: 95 - DNA, 42 bp, Artificial sequence
  Synthetic
gtcaaagggatatattttgggataaaatactcagtttctttc

SEQ ID NO: 96 - DNA, 42 bp, Artificial sequence
  Synthetic
gaaagaaactgagtattttatcccaaaatatatccctttgac

SEQ ID NO: 97 - DNA, 25 bp, Artificial sequence
  Synthetic
taatgaggcaagtccagtcccgctt

SEQ ID NO: 98 - DNA, 25 bp, Artificial sequence
  Synthetic
aaagaggggataatgaggcaagtcc

SEQ ID NO: 99 - DNA, 25 bp, Artificial sequence
  Synthetic
gggataatgaggcaagtccagtccc

SEQ ID NO: 100 - DNA, 25 bp, Artificial sequence
  Synthetic
aaagaggggataatgaggcaagtcc

SEQ ID NO: 101 - DNA, 60 bp, Homo sapiens
ggctatttcaaaatatgtcaaagggatatattttgagataaaatactcagtttctttcag

SEQ ID NO: 102 - DNA, 59 bp, Homo sapiens
gtagacggctgagctcagccgacgtggggtcatgtcctgtttacacagggaaggtgaag

SEQ ID NO: 103 - DNA, 119 bp, Homo sapiens
ttctcggtggaacttaaaatgctgtgagacaccagacagacagatactgtgaacttggagctctctaatgaagggataccaaagtcttgtattcaatttttttttccttaaattgtcag

SEQ ID NO: 104 - DNA, 173 bp, Homo sapiens
ggtttgctgactcacagaaacacaacagaacagacttaaaggagagtgggagaaaaggaaactcgatgagaggatcaggcagcgcctgagggtagatgttaagagcactccatgcccgaaagccctctgtattccctgcttcgggccccagtctggccattcctagcaacaaa

SEQ ID NO: 105 - DNA, 100 bp, Homo sapiens
gagacgttctagacccagtgtcttcagtggaaacagagcacaggctgtctgcagtctcaggcttcctagcagccctgataagagaagtagagcaatgcag

SEQ ID NO: 106 - DNA, 76 bp, Homo sapiens
atgggctatctgaagaggaacggggacgggagcctgctctacagcgtggtcaacacggccgagccggacgctgatg

SEQ ID NO: 107 - DNA, 160 bp, Homo sapiens
tttctttcagggcctgccatctatcacgtgatgctgtactaaagtcggatcggaatttggtgtcttattaccacggagtctgctttgtcaaccttcagatttctgttttaaagttaatgctggtcagctgtgcctgaattccaaaagaggggataatgag

(19) United States
(12) Patent Application Publication
(10) Pub. No.: US 2023/0159920 A1
(43) Pub. Date: May 25, 2023

(54) METHODS AND COMPOSITIONS COMPRISING BRD9 ACTIVATING THERAPIES FOR TREATING CANCERS AND RELATED DISORDERS

(71) Applicants: Fred Hutchinson Cancer Research Center, Seattle, WA (US); Memorial Sloan Kettering Cancer Center, New York, NY (US)

(72) Inventors: Robert K. Bradley, Seattle, WA (US); Omar Abdel-Wahab, New York, NY (US); Daichi Inoue, New York, NY (US)

(73) Assignees: Fred Hutchinson Cancer Research Center, Seattle, WA (US); Memorial Sloan Kettering Cancer Center, New York, NY (US)

(21) Appl. No.: 17/621,429

(22) PCT Filed: Jun. 25, 2020
(86) PCT No.: PCT/US2020/039645

§ 371 Date: Dec. 21, 2021

Related U.S. Application Data
(60) Provisional application No. 62/867,055, filed on Jun. 26, 2019.

(51) Int. Cl. C12N 15/113; A61P 35/00; A61K 38/17

(52) U.S. Cl. CPC C12N 15/113; A61P 35/00; A61K 38/1709; C12N 2310/11

(57) ABSTRACT

The current disclosure relates to methods and compositions for increasing functional expression of BRD9 in a cell. The methods and compositions can be incorporated into methods for treating cancer through the administration of BRD9 activating therapies. Accordingly, aspects of the disclosure relate to compositions and methods for treating cancer, a pre-malignant disease, or a dysplastic disease in a subject. The method can comprise administering a BRD9 activating therapy to the subject.

### CROSS-REFERENCE TO RELATED APPLICATION

This application claims the benefit of U.S. Application No. 62/867,055, filed Jun. 26, 2019, the disclosure of which is hereby expressly incorporated by reference in its entirety herein.

### STATEMENT OF GOVERNMENT LICENSE RIGHTS

This invention was made with Government support under grant numbers HL128239, DK103854, and CA218896 awarded by the National Institutes of Health and W81XWH-12-1-0041 awarded by the U.S. Department of the Army. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 72082_Sequence_Final_2020-06-24.txt. The text file is 87 KB; was created on Jun. 24, 2020; and is being submitted via EFS-Web with the filing of the specification.

### FIELD OF THE INVENTION

The disclosure generally relates to the field of medicine. More particularly, it concerns compositions and methods involving nucleic acids and polypeptides for treating cancers and related disorders.

### BACKGROUND

SF3B1 encodes an RNA splicing factor that recognizes 3′ splice sites (3′ ss) and is the most commonly mutated spliceosomal gene in cancer. SF3B1 is recurrently mutated in myeloid and lymphoid leukemias as well as solid tumors, at rates of up to 14-29% (uveal melanoma (UVM)) and 65-83% (refractory anemia with ring sideroblasts). SF3B1 mutations are concentrated at specific residues, frequently in a cancer type-dependent manner, and appear as both founder lesions and later events, including in response to therapeutic challenge.

SF3B1 encodes a core component of the U2 small nuclear ribonucleoprotein (snRNP) complex that binds upstream of intronic branchpoints to facilitate subsequent 3′ ss recognition. Consistent with SF3B1's normal role in splicing, several studies reported that SF3B1 mutations alter branchpoint selection to induce widespread usage of abnormal 3′ ss. Although these and other studies identified many mis-spliced genes in patient samples and murine models of SF3B1 mutations, few functional links between SF3B1 mutations and disease phenotypes are defined.

One central barrier to identifying precise connections between SF3B1 mutation-induced mis-splicing and disease is the multitude of mis-spliced genes in SF3B1-mutant cells.

Accordingly, despite the advances the study of SF3B1, a need remains to understand the mechanistic role of SF3B1-mediated mis-splicing, and for effective therapeutics to address the resultant diseases. The present disclosure addresses these and related needs.

### SUMMARY OF THE INVENTION

The current disclosure relates to compositions and methods that increase functional expression of BRD9 in a cell. The compositions and methods as disclosed herein provide functionality for enhancing or recovering non-canonical BAF formation, which can have implications for downstream gene activation profiles with profound effects on cancer or tumor suppression.

Thus, in one aspect, the disclosure provides methods for increasing functional expression of BRD9 in a cell, comprising contacting the cell with an effective amount of a BRD9 activating agent. The cell can be in vivo or in vitro.

In another aspect the disclosure provides a method for treating cancer, a pre-malignant disease, or a dysplastic disease in a subject, the method comprising administering a BRD9 activating therapy or an antisense oligo to the subject.

A further aspect of the disclosure relates to a method for treating cancer, a pre-malignant disease, or a dysplastic disease in a subject, the method comprising increasing non-canonical BAF formation in the subject.

In some embodiments, the subject has been diagnosed with cancer, a pre-malignant disease, or a dysplastic disease. In some embodiments, the cancer or disease is a SF3B1-mutant cancer or disease. In some embodiments, the subject has and/or has been diagnosed with a cancer or disease selected from blood cancer, bladder cancer, uveal melanoma, cutaneous cancer, pancreatic cancer, breast cancer, prostate cancer, genitourinary cancer, myeloid cancer, lymphoid cancer, myelodysplastic syndrome, and pre-malignant myeloid disease, and the like.

In some embodiments, the subject (or cell) has been determined to have a SF3B1 mutation. In some embodiments, the SF3B1 mutation comprises K700E. In some embodiments, the SF3B1 mutation comprises R625H. In some embodiments, the SF3B1 mutation comprises E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, M971V, or combinations thereof. In some embodiments, the SF3B1 mutation comprises a gain of function mutation.

In some embodiments, the BRD9 activating therapy comprises administering a BRD9 polypeptide or nucleic acid encoding a BRD9 polypeptide. In some embodiments, the BRD9 activating therapy comprises a therapy that activates transcription of the endogenous BRD9 gene. In some embodiments, the BRD9 activating therapy comprises a splicing modifier. In some embodiments, the splicing modifier comprises an antisense nucleic acid having base complementarity with a BRD9 splice site. In some embodiments, the antisense molecule binds to the branch point, 5′ splice site, or 3′ splice site of the poison exon, exon 14a. In some embodiments, the splicing modifier mutates the branch point, 5′ splice site, or 3′ splice site of the poison exon, exon 14a. In some embodiments, the splicing modifier mutates the exonic splicing enhancer of the poison exon, exon 14a. In some embodiments, the splicing modifier comprises a modified nucleic acid. In some embodiments, the splicing modifier comprises a morpholino. Exemplary morpholinos include those that target the nucleic acid sequences of BRD9 corresponding to SEQ ID NOS:4-6 and 8-12.

In some embodiments, the splicing modifier comprises a non-specific inhibitor of splice site recognition. In some embodiments, the splicing modifier comprises a compound. In some embodiments, the splicing modifier comprises a pladienolide (A-G, described in Sakai T, et al. Pladienolides, new substances from culture of Streptomyces platensis Mer-11107. I. Taxonomy, fermentation, isolation and screening. J Antibiot (Tokyo) 2004; 57:173-179, which is herein incorporated by reference), Herboxiedene (GEX1A and other GEX family members), 6-norherboxidiene, FR901463, FR901464, FR901465, Meayamycin, Spliceostatin A, (methylated derivative of FR901464), TG003, SRPIN340, Cpd-1 polypeptide, Cpd-2 polypeptide, Cpd-391 polypeptide, or the like.

In some embodiments, the subject has previously been treated for the disorder. In some embodiments, the subject has been determined to be non-responsive to the previous therapy. In some embodiments, the subject has been determined to experience toxicity from the previous therapy. In some embodiments, the cancer or disorder is classified as refractory or recurrent.

In some embodiments, the subject is a human. In some embodiments, the subject is non-human. In some embodiments, the subject is a rat, mouse, rabbit, cat, dog, pig, or horse.

In some embodiments, the method further comprises administration of an additional therapy. In some embodiments, the additional therapy comprises an immunotherapy. In some embodiments, the additional therapy comprises a BRD9 polypeptide or a nucleic acid encoding a BRD9 polypeptide. The BRD9 polypeptide may comprise the bromodomain and/or the DUF3512 domain.

In some embodiments, the BRD9 activating therapy is administered by intravenous injection. In some embodiments, the BRD9 activating therapy reconstitutes or restores ncBAF formation and/or reduces or prevents BRD9 mis-splicing.

In additional aspects, the disclosure provides an antisense oligonucleotide comprising at least 10 contiguous nucleotides of a nucleic acid selected from SEQ ID NOS:4-6 and 8-12, or the complement thereof. In some embodiments, the antisense oligonucleotide comprises at least one modified nucleotide. In some embodiments, the antisense oligonucleotide comprises a modified backbone. In some embodiments, the antisense oligonucleotide comprises at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 modified oligonucleotides (or any range drivable therein). In some embodiments, the antisense oligonucleotide comprises a morpholino. In some embodiments, the antisense oligonucleotide comprises a nucleic acid sequence with at least 90% sequence identity to a nucleic acid selected from: SEQ ID NOS:4-6 and 8-12, a complement of SEQ ID NOS:4-6 and 8-12, and a fragment of SEQ ID NOS:4-6 and 8-12. In some embodiments, the antisense oligonucleotide comprises a nucleic acid sequence with at least 75, 76, 77, 78, 79, 80, 81, 82, 83, 84, 85, 86, 87, 88, 89, 90, 91, 92, 93, 94, 95, 96, 97, 98, 99, or 100% sequence identity to a nucleic acid selected from: SEQ ID NOS:4-6 and 8-12, a complement of SEQ ID NOS:4-6 and 8-12, and a fragment of SEQ ID NOS:4-6 and 8-12.

Other objects, features and advantages of the present invention will become apparent from the following detailed description. It will be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1H. BRD9 mis-splicing causes BRD9 loss and proliferative advantage in SF3B1-mutated cancers. FIG. 1A is a heatmap illustrating unsupervised clustering of patient samples on the basis of events that are differentially spliced in UVM (MEL270) and myeloid leukaemia (K562) cells that express SF3B1^{K700E} versus wild-type (WT) SF3B1. a3ss, alternative 3′ splice site; CLL, chronic lymphocytic leukaemia (data are from Darman, R. B. et al. Cancer-associated SF3B1 hotspot mutations induce cryptic 3′ splice site selection through use of a different branch point. Cell Rep. 13,1033-1045 (2015)); MDS, myelodysplastic syndromes (data are from Pellagatti, A. et al. Impact of spliceosome mutations on RNA splicing in myelodysplasia: dysregulated genes/pathways and clinical associations. Blood 132, 1225-1240 (2018)); mxe, mutually exclusive exons; PSI, percentage spliced in (fraction of mRNA that corresponds to the mutant SF3B1-promoted isoform), with per-event and per-cohort range normalization; ri, retained intron; se, skipped exon; TCGA, The Cancer Genome Atlas. Data for UVM are from Alsafadi, S. et al. Cancer-associated SF3B1 mutations affect alternative splicing by promoting alternative branchpoint usage. Nat. Commun. 7, 10615 (2016) (middle) or the TCGA (right). FAM192A, HRSP12, MTERFD3, NPIP and UQCC are also known as PSME3IP1, RIDA, MTERF2, NPIPA1 and UQCC1, respectively. FIG. 1B is a Venn diagram illustrating genes for which mutant SF3B1 promotes an isoform predicted to trigger NMD (alternative 3′ splice site and skipped exon events only) in one or more cohorts. FIG. 1C is a schematic of a CRISPR-Cas9-based positive-selection screen targeting genes for which mutant SF3B1 promotes an isoform predicted to trigger NMD. FIG. 1D is a per-gene scatter plot comparing CRISPR screen enrichment (y axis) to differential splicing in TCGA cohort of patients with UVM (x axis). Pten was used as a positive control. n=6 biologically independent experiments. Per-gene significance computed with two-sided correlation-adjusted mean rank gene set (CAMERA) test. The false-discovery rate (FDR) was computed using the Benjamini-Hochberg method. FIG. 1E is a bar graph illustrating estimated poison exon inclusion isoform half-life based on qRT-PCR data. qRT-PCR measurements were made over 8 hours to determine the half-lives of the poison exon inclusion and exclusion isoforms in isogenic K562 SF3B1^{K700E} cells treated with shUPF1 #1 and shUPF1 #2 and actinomycin D to inhibit transcription. NMD inhibition via UPF1 knockdown stabilizes the inclusion, but not exclusion, isoform. n=2 biologically independent experiments and n=2 technically independent experiments for the inclusion isoform; n=3 technically independent experiments for the exclusion isoform. P value was calculated by two-sided t-test at 8 h. FIG. 1F is a bar graph illustrating estimated poison exon exclusion isoform half-life based on qRT-PCR data. The conditions are described above for FIG. 1E. FIG. 1G is a bar graph illustrating estimated poison exon inclusion isoform half-life based on qRT-PCR data, with the same conditions as described for FIG. 1E except using NALM-6 SF3B1^{K700E} cells. FIG. 1H is a bar graph illustrating estimated poison exon exclusion isoform half-life based on qRT-PCR data, with the same conditions as described for FIG. 1F except using NALM-6 SF3B1^{K700E} cells.

FIGS. 2A-2D. Mutant SF3B1 recognizes an aberrant, deep intronic branchpoint within BRD9. FIG. 2A is a schematic of BRD9 gene structure and protein domains. Inset illustrates the branchpoints used when the poison exon is included (top; the sequence is set forth as SEQ ID NO:101 or excluded) (bottom; the sequence is set forth as SEQ ID NO: 102). Single and double underlining indicates sequence motifs that were subsequently mutated. aa, amino acid. FIG. 2B illustrates PCR with reverse transcription (RT-PCR) analysis of inclusion of the BRD9 poison exon in a minigene (top) or endogenous (bottom) context, following transfection of minigenes with the illustrated mutations into MEL270 cells with doxycycline (dox)-inducible Flag-SF3B1(WT) or Flag-SF3B1(K700E). Representative images from n=3 biologically independent experiments. Native, no mutations. Similar results were observed using T47D cells with doxycycline-inducible Flag-tagged SF3B1(K700E) (not shown). FIG. 2C illustrates PCR with reverse transcriptase analysis, as described for FIG. 2B except minigene mutations (shown in light gray and underlined) at the 5′ end of the poison exon. ESE, exonic splicing enhancer. Similar results were observed when assaying minigene mutagenesis in T47D cells with doxycycline-inducible Flag-tagged SF3B1(K700E) (not shown). FIG. 2D illustrates RT-PCR (top) illustrating the loss of inclusion of the BRD9 poison exon, and corresponding western blot (bottom) in MEL202 (SF3B1^{R625G}) clones following CRISPR-Cas9 targeting of the poison exon. Indels were sequenced for confirmation (not shown). Representative images from n=2 (RT-PCR) and n=3 (western blot) biologically independent experiments.

FIG. 3A-3B. BRD9 loss perturbs the formation and localization of the ncBAF complex. FIG. 3A is a schematic of non-canonical BAF (ncBAF; left), canonical BAF (cBAF; middle) and polybromo-associated BAF (PBAF; right) complexes. Solidus denotes one of the proteins is present; comma denotes one or more of the proteins are present or that mutually exclusive inclusion of proteins may occur. FIG. 3B shows the immunoprecipitation with GLTSCR1 or BRG1 antibody followed by immunoblotting in MEL270 cells that express exogenous SF3B1(K700E) (left) or were treated with BRD9 degrader19 (dBRD9) (right). Representative images from n=3 biologically independent experiments.

FIGS. 4A-4N. BRD9 is a therapeutically targetable tumour suppressor in melanoma. FIG. 4A graphically illustrates BRD9 expression (z-score normalized) in TCGA UVM samples with (n=18) or without (n=62) SF3B1 mutations. P value calculated by two-sided t-test. FIG. 4B graphically illustrates tumour volume 49 days after subcutaneous engraftment of Melan-a cells transduced with the indicated shRNAs into SCID mice. n=16, 16, 16, 14 and 14 tumours per group (left to right). Error bars, mean±s.d. P values calculated by two-sided t-test. FIG. 4C graphically illustrates the numbers of pulmonary B16 metastases identified in 14 days after intravenous injection of B16 cells with or without Brd9 shRNA (MLS-E vector). FIG. 4D graphically illustrates the relative percentages of GFP+92.1 cells with or without BRD9 shRNA (MLS-E vector), assessed by flow cytometric analysis of lung tissue in recipient NSG (NOD-SCID Il2rg−/−) mice 14 days after intravenous injection by tail vein. The signal was normalized by dividing by the average percentage of GFP+ cells in the Renilla shRNA (control) group. n=5 biologically independent experiments per group. P value was calculated relative to the Renilla shRNA group by a two-sided t-test. FIG. 4E graphically illustrates the tumor volume from tumours derived from transplantation of Melan-a cells transduced with doxycycline-inducible Brd9 shRNA. Doxycycline was administered for nine weeks and followed by doxycycline withdrawal for three weeks. FIG. 4F graphically illustrates the survival of SCID mice engrafted with MEL270 cells that express empty vector, full-length wild-type BRD9 or a BRD9 bromodomain-deletion mutant (ABD). n=5 mice per group. P value calculated by log-rank test. FIG. 4G graphically illustrates tumour volume from experiments shown in FIG. 4F, 21 days after engraftment. n=10 tumours per group. Error bars, mean±s.d. P values calculated by two-sided t-test. FIG. 4H graphically illustrates HTRA1 expression in samples from patients in the TCGA UVM cohort with (n=18) or without (n=62) SF3B1 mutations. Expression is z-score normalized across all samples. Data are presented as mean±s.d. P value computed with two-sided t-test. FIG. 4I graphically illustrates colony number (left) of MEL202 cells (SF3B1R625G) without (control) or with (clone 1, clone 2 and clone 3) CRISPR-Cas9-induced disruption of the BRD9 poison exon. Indels are illustrated in Extended Data FIG. 2o. n=3 biologically independent experiments. Error bars, mean±s.d. P values calculated by two-sided t-test at day 3 (right). FIG. 4J graphically illustrates tumour volume (left) of mice engrafted with control or clone 1 cells from FIG. 4Id. n=6 tumours per group. Error bars, mean±s.d. P value calculated by two-sided t-test at week 7. FIG. 4K illustrates a schematic of select ASO designs (top), and their effects as determined by RT-PCR (middle) and western blot (bottom) for BRD9. MEL202 cells (SF3B1R625G) were treated with a non-targeting (control) or targeting morpholino at 10 μM for 24 h. Representative images from n=3 biologically independent experiments. FIG. 4L graphically illustrates growth of MEL202 cells (SF3B1^{R625G}) treated with 10 μM of control non-targeting (control) or BRD9 poison-exon-targeting morpholinos (no. 3, no. 6 and no. 7). n=3 biologically independent experiments per group. P values at day 9 were calculated relative to the control group by a two-sided t-test. FIG. 4M graphically illustrates tumour weight following 16 days of in vivo treatment of MEL202-derived xenografts (SF3B1R625G) with PBS or a non-targeting (control) or poison-exon targeting (no. 6) morpholino (12.5 mg kg-1, every other day to a total of 8 intratumoral injections). n=10 tumours per group. Error bars, mean±s.d. P values calculated by two-sided t-test. FIG. 4N graphically illustrates tumour weight following in vivo morpholino treatment of a patient-derived rectal melanoma xenograft (SF3B1R625C). n=5 tumours per group. P value calculated by two-sided t-test. Error bars, mean±s.d.

FIGS. 5A-5B. Use of multiple, distinct NMD isoforms of BRD9. FIG. 5A is a schematic BRD9 gene structure illustrating constitutive BRD9 exons and alternative splicing events that are predicted to induce NMD. SF3B1 mutations promote inclusion of the BRD9 poison exon in intron 14. FIG. 5B is a table showing the genomic coordinates (hg19/GRCh37 assembly) of each NMD-inducing event illustrated in FIG. 5A, as well as genomic sequence of each alternatively spliced region noted in FIG. 5A. Select sequences are set forth herein as SEQ ID NO:103 (intron 3 a3ss), SEQ ID NO:104 (intron 5 se), SEQ ID NO:105 (intron 8 se), SEQ ID NO:106 (exon 9 se), and SEQ ID NO:107 (intron 14 se). The third column indicates the specific isoform that is a predicted NMD substrate. Prox, intron-proximal competing 3′ splice site; dist, intron-distal competing 3′ splice site; inc, exon inclusion; exc, exon exclusion.

### DETAILED DESCRIPTION OF THE INVENTION

This disclosure is based on the inventors' investigation of tumorigenic effects of splicing factor 3B subunit 1 (SF3B1) mutations. As described in more detail below, the inventors integrated pan-cancer RNA sequencing to identify mutant SF3B1-dependent aberrant splicing with a positive enrichment CRISPR screen to prioritize splicing alterations that functionally promote tumorigenesis. The inventors determined that diverse, recurrent SF3B1 mutations converge on repression of BRD9, a core component of the recently described non-canonical BAF (ncBAF) complex. Mutant SF3B1 recognizes an aberrant deep intronic branchpoint within BRD9, thereby inducing inclusion of an endogenous retrovirus-derived poison exon and subsequent BRD9 mRNA degradation. BRD9 depletion causes loss of ncBAF at CTCF-bound loci and promotes melanomagenesis. The inventors demonstrated that BRD9 is a potent tumor suppressor in uveal melanoma, such that correcting BRD9 mis-splicing in SF3B1-mutant cell lines and patient-derived melanoma xenografts with antisense oligonucleotides (ASOs) or by directly targeting its poison exon with CRISPR-directed mutagenesis profoundly suppresses tumor growth. The results indicate that ncBAF is disrupted in the diverse malignancies characterized by SF3B1 mutations, identify a single aberrant splicing event that functionally contributes to the pathogenesis of SF3B1-mutant cancers, and suggest a mechanism-based therapeutic for these malignancies.

In view of the foregoing, aspects of this disclosure relate to compositions and related methods that restore or increase expression of BRD9 and the consequent formation of ncBAF in cells. These compositions and methods, described in more detail below, facilitate the treatment of cancer, pre-malignant diseases, or dysplastic diseases, among other useful applications.

The term “cancer” refers to a condition characterized by one or more cells that exhibit autonomous, unregulated growth, such that they exhibit an aberrant growth phenotype. The growth phenotype can be characterized by a significant loss of control over cell proliferation and tend to form aggregations or tumors with unique microenvironments compared to healthy tissues. Cells of interest for detection, analysis, or treatment in the present application include precancerous (e.g., benign), malignant, pre-metastatic, metastatic, and non-metastatic cells. Various types of cancers encompassed by the present disclosure are described in more detail below. The term “pre-malignant” disease is a condition or lesion involving abnormal cells that are associated with an increased risk of developing into cancer. Examples of pre-malignant disease include colon polyps, which can progress into colon cancer, monoclonal gammopathy of undetermined significance, which can progress into multiple myeloma or myelodysplastic syndrome, and cervical dysplasia, which can progress into cervical cancer. The term “dysplastic disease” relates to an abnormality of development or an anomaly of growth and differentiation in a tissue.

BRD9 Activating Compositions

An illustrative genomic sequence of the BRD9 gene comprises SEQ ID NO:1. The exons in the illustrative full length BRD9 gene are encoded by the sequences at nucleotide positions 1-219, 971-1185, 1538-1670, 1754-1757, 3178-3238, 3660-3804, 5354-5464, 6118-6233, 8755-8887, 11643-11718, 12936-13031, 14338-14470, 16613-16724, 21261-21299, 22250-22352, 27244-27411, and 28257-29090 of SEQ ID NO:1. The aberrant exon caused by alternative splicing resulting from mutated SF3B1, also referred to herein as the “poison exon”, is encoded by the sequence at nucleotide positions 23421-23580.

The human BRD9 wildtype protein is exemplified by the amino acid sequence set forth as SEQ ID NO:2. An exemplary human BRD9 cDNA is represented by the sequence set forth in SEQ ID NO: 3. SEQ ID NO: 7 is an exemplary sequence corresponding to the intron and exon 14a (i.e., the poison exon) and a portion of the intron downstream of the poison exon sequence.

In one aspect, the present disclosure provides compositions that can disrupt alternative splicing of the BRD9 gene and, thus, prevent inclusion of the poison exon (i.e., exon 14a, corresponding to nucleotide positions 23421-23580 of SEQ ID NO:1) into the functional mRNA. These compositions are generally referred to as “splicing modifiers”.

In some embodiments, the splicing modifier is an antisense oligonucleotide, also referred to as an “ASO”. In some embodiments, the ASO can comprise, consist essentially of, or consist of a nucleic acid sequence that is at least 80% identical to 10 contiguous nucleotides of one of SEQ ID NOS:4-6 and 8-12, or a complement of one of SEQ ID NOS:4-6 and 8-12. In some embodiments, the ASO comprises a nucleic acid sequence with at least 75, 76, 77, 78, 79, 80, 81, 82, 83, 84, 85, 86, 87, 88, 89, 90, 91, 92, 93, 94, 95, 96, 97, 98, 99, or 100% sequence identity to a nucleic acid selected from: SEQ ID NOS:4-6 and 8-12, a complement of SEQ ID NOS:4-6 and 8-12, and a fragment of SEQ ID NOS:4-6, 8-12, and 97-100. In some embodiments, the ASO can comprise at least one modified nucleotide, e.g., a nucleotide with a modification of a canonical nucleobase. In some embodiments, the ASO comprises a morpholino contains DNA bases attached to a backbone of methylenemorpholine rings linked through phosphorodiamidate groups. Morpholinos block access of other molecules to small (˜25 base) specific sequences of the base-pairing surfaces of ribonucleic acid (RNA) and can result in knock down of the target gene function. In some embodiments, the ASO comprises a nucleic acid sequence with at least 90% sequence identity to a nucleic acid selected from: SEQ ID NOS:4-6 and 8-12, a complement of SEQ ID NOS:4-6 and 8-12, and a functional fragment of SEQ ID NOS:4-6 and 8-12. The term “functional fragment” refers to a fragment with sufficient length and sequence to bind to a portion of the target sequence (e.g., poison exon) so as to prevent its incorporation into an mRNA transcript.

In some embodiments, the splicing modifier disrupts the branch point (nucleotide 1054 of SEQ ID NO:7). As used in this context, the term “disrupts” refers to disruption or prevention of the mis-splicing occurring due to recognition of the branch point by the spliceosome. Depending on the mechanism, the disruption can occur by the splicing modifier binding to the branch point or otherwise blocking or preventing recognition of the branchpoint by the spliceosome (e.g., by an ASO), or it can occur by mutating the branchpoint such that the spliceosome can no longer recognize or bind to the sequence at that location (e.g., by an editing mechanism such as CRISPR/Cas9), thus preventing or disrupting the mis-splicing. In some embodiments, the splicing modifier comprises an antisense oligonucleotide comprising at least 10 contiguous nucleotides of (SEQ ID NO:8) or at least 10 nucleotides of a complement of SEQ ID NO:8. In some embodiments, the splicing modifier comprises an antisense oligonucleotide comprising a fragment of SEQ ID NO:8, wherein the fragment comprises the branch point (nucleotide 31 of SEQ ID NO:8) or the complement of a fragment of SEQ ID NO:8 comprising the complement of the branch point. In some embodiments, the splicing modifier mutates nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or 61 (or combinations thereof) of the genomic DNA corresponding to SEQ ID NO:8 of the BRD9 gene. In some embodiments, the splicing modifier mutates nucleotide 31 of the genomic DNA corresponding to SEQ ID NO:8 of the BRD9 gene. In some embodiments, the splicing modifier mutates or comprises an antisense oligo that anneals to the donor/acceptor site (nucleotides 1-2 of SEQ ID NO:7). In some embodiments, the splicing modifier prevents recognition of the donor site. In some embodiments, the splicing modifier prevents recognition of the 3′ splice site (nucleotides 1067-1068 of SEQ ID NO:7). In some embodiments, the splicing modifier prevents recognition of the region adjacent to the 3′ splice site. In some embodiments, the region adjacent to the 3′ splice site comprises nucleotides that are within about 1, 2, 3, 4, 5, 6, 7, 8, 9, 10, or more nucleotide positions 5′ or 3′ to nucleotide positions 1067-1068 of SEQ ID NO:7. In some embodiments, the splicing modifier comprises an antisense oligonucleotide comprising at least 10 contiguous nucleotides of (SEQ ID NO:9) or at least 10 contiguous nucleotides of a complement of SEQ ID NO:9. In some embodiments, the splicing modifier comprises an antisense oligonucleotide comprising a fragment of SEQ ID NO:9, wherein the fragment comprises the 3′ splice site (nucleotides 31-32 of SEQ ID NO:9) or the complement of a fragment of SEQ ID NO:9 comprising the complement of the 3′ splice site. In some embodiments, the fragment comprises at least one nucleotide of the 3′ splice site. In some embodiments, the splicing modifier mutates nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 (or combinations thereof) of the genomic DNA corresponding to SEQ ID NO:9 of the BRD9 gene. In some embodiments, the splicing modifier blocks or prevents recognition of the poison exon exonic enhancer (nucleotides 1071-1073 of SEQ ID NO:7). For example, the splicing modifier can mutate at least 1, 2, or 3 nucleotides of the exonic enhancer. In some embodiments, the splicing modifier blocks or prevents recognition of the region adjacent to the exonic enhancer or at least 1, 2, 3, 4, 5, 6, or 7 nucleotides in the 5′ or 3′ adjacent region of the exonic enhancer. In some embodiments, the splicing modifier comprises an antisense oligonucleotide comprising at least 10 contiguous nucleotides of (SEQ ID NO:10) or at least 10 contiguous nucleotides of a complement of SEQ ID NO:10. In some embodiments, the splicing modifier comprises an antisense oligonucleotide comprising a fragment of SEQ ID NO:10, wherein the fragment comprises the exonic enhancer (nucleotides 31-33 of SEQ ID NO:10) or the complement of a fragment of SEQ ID NO:10 comprising the complement of the exonic enhancer. In some embodiments, the splicing modifier mutates nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 (or combinations thereof) of the genomic DNA corresponding to SEQ ID NO:10 of the BRD9 gene. In some embodiments, the splicing modifier blocks or prevents recognition of the 5′ splice site (nucleotides 1229-1230 of SEQ ID NO:7). In some embodiments, the splicing modifier blocks or prevents recognition of the region adjacent to the 5′ splice site. In some embodiments, the splicing modifier comprises an antisense oligonucleotide comprising at least 10 contiguous nucleotides of (SEQ ID NO:11) or at least 10 contiguous nucleotides of a complement of SEQ ID NO:11. In some embodiments, the splicing modifier comprises an antisense oligonucleotide comprising a fragment of SEQ ID NO:11, wherein the fragment comprises the 5′ splice site (nucleotides 31-32 of SEQ ID NO:11) or the complement of a fragment of SEQ ID NO:11 comprising the complement of the 5′ splice site. In some embodiments, the fragment comprises at least one nucleotide of the 5′ splice site. In some embodiments, the splicing modifier mutates nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 (or combinations thereof) of the genomic DNA corresponding to SEQ ID NO: 11 of the BRD9 gene. In some embodiments, the antisense oligonucleotide comprises a fragment of (SEQ ID NO:12) or a fragment of a complement of SEQ ID NO:12.

In some embodiments, the BRD9 polypeptide or nucleic acid comprises a human BRD9 polypeptide or human BRD9 nucleic acid. In some embodiments, the BRD9 polypeptide or BRD9 nucleic acid is non-human. In some embodiments, the BRD9 polypeptide or BRD9 nucleic acid is from or derived from, e.g., mouse, rat, cow, horse, dog, rabbit, guinea pig, or goat.

The polypeptides or polynucleotides of the disclosure such as those comprising or encoding for a BRD9 polypeptide or an antisense molecule, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 or more contiguous amino acids or nucleic acids, or any range derivable therein, of SEQ ID NOS:1-17 or of a nucleic acid that is complementary to one of SEQ ID NOS:1 and 3-17, or of a nucleic acid that encodes a protein comprising an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:2.

The polypeptides or polynucleotides of the disclosure such as those comprising or encoding for a BRD9 polypeptide or antisense molecule, may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117, 1118, 1119, 1120, 1121, 1122, 1123, 1124, 1125, 1126, 1127, 1128, 1129, 1130, 1131, 1132, 1133, 1134, 1135, 1136, 1137, 1138, 1139, 1140, 1141, 1142, 1143, 1144, 1145, 1146, 1147, 1148, 1149, 1150, 1151, 1152, 1153, 1154, 1155, 1156, 1157, 1158, 1159, 1160, 1161, 1162, 1163, 1164, 1165, 1166, 1167, 1168, 1169, 1170, 1171, 1172, 1173, 1174, 1175, 1176, 1177, 1178, 1179, 1180, 1181, 1182, 1183, 1184, 1185, 1186, 1187, 1188, 1189, 1190, 1191, 1192, 1193, 1194, 1195, 1196, 1197, 1198, 1199, 1200, 1201, 1202, 1203, 1204, 1205, 1206, 1207, 1208, 1209, 1210, 1211, 1212, 1213, 1214, 1215, 1216, 1217, 1218, 1219, 1220, 1221, 1222, 1223, 1224, 1225, 1226, 1227, 1228, 1229, 1230, 1231, 1232, 1233, 1234, 1235, 1236, 1237, 1238, 1239, 1240, 1241, 1242, 1243, 1244, 1245, 1246, 1247, 1248, 1249, 1250, 1251, 1252, 1253, 1254, 1255, 1256, 1257, 1258, 1259, 1260, 1261, 1262, 1263, 1264, 1265, 1266, 1267, 1268, 1269, 1270, 1271, 1272, 1273, 1274, 1275, 1276, 1277, 1278, 1279, 1280, 1281, 1282, 1283, 1284, 1285, 1286, 1287, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, 1549, 1550, 1551, 1552, 1553, 1554, 1555, 1556, 1557, 1558, 1559, 1560, 1561, 1562, 1563, 1564, 1565, 1566, 1567, 1568, 1569, 1570, 1571, 1572, 1573, 1574, 1575, 1576, 1577, 1578, 1579, 1580, 1581, 1582, 1583, 1584, 1585, 1586, 1587, 1588, 1589, 1590, 1591, 1592, 1593, 1594, 1595, 1596, 1597, 1598, 1599, 1600, 1601, 1602, 1603, 1604, 1605, 1606, 1607, 1608, 1609, 1610, 1611, 1612, 1613, 1614, 1615, 1616, 1617, 1618, 1619, 1620, 1621, 1622, 1623, 1624, 1625, 1626, 1627, 1628, 1629, 1630, 1631, 1632, 1633, 1634, 1635, 1636, 1637, 1638, 1639, 1640, 1641, 1642, 1643, 1644, 1645, 1646, 1647, 1648, 1649, 1650, 1651, 1652, 1653, 1654, 1655, 1656, 1657, 1658, 1659, 1660, 1661, 1662, 1663, 1664, 1665, 1666, 1667, 1668, 1669, 1670, 1671, 1672, 1673, 1674, 1675, 1676, 1677, 1678, 1679, 1680, 1681, 1682, 1683, 1684, 1685, 1686, 1687, 1688, 1689, 1690, 1691, 1692, 1693, 1694, 1695, 1696, 1697, 1698, 1699, 1700, 1701, 1702, 1703, 1704, 1705, 1706, 1707, 1708, 1709, 1710, 1711, 1712, 1713, 1714, 1715, 1716, 1717, 1718, 1719, 1720, 1721, 1722, 1723, 1724, 1725, 1726, 1727, 1728, 1729, 1730, 1731, 1732, 1733, 1734, 1735, 1736, 1737, 1738, 1739, 1740, 1741, 1742, 1743, 1744, 1745, 1746, 1747, 1748, 1749, 1750, 1751, 1752, 1753, 1754, 1755, 1756, 1757, 1758, 1759, 1760, 1761, 1762, 1763, 1764, 1765, 1766, 1767, 1768, 1769, 1770, 1771, 1772, 1773, 1774, 1775, 1776, 1777, 1778, 1779, 1780, 1781, 1782, 1783, 1784, 1785, 1786, 1787, 1788, 1789, 1790, 1791, 1792, 1793, 1794, 1795, 1796, 1797, 1798, 1799, 1800, 1801, 1802, 1803, 1804, 1805, 1806, 1807, 1808, 1809, 1810, 1811, 1812, 1813, 1814, 1815, 1816, 1817, 1818, 1819, 1820, 1821, 1822, 1823, 1824, 1825, 1826, 1827, 1828, 1829, 1830, 1831, 1832, 1833, 1834, 1835, 1836, 1837, 1838, 1839, 1840, 1841, 1842, 1843, 1844, 1845, 1846, 1847, 1848, 1849, 1850, 1851, 1852, 1853, 1854, 1855, 1856, 1857, 1858, 1859, 1860, 1861, 1862, 1863, 1864, 1865, 1866, 1867, 1868, 1869, 1870, 1871, 1872, 1873, 1874, 1875, 1876, 1877, 1878, 1879, 1880, 1881, 1882, 1883, 1884, 1885, 1886, 1887, 1888, 1889, 1890, 1891, 1892, 1893, 1894, 1895, 1896, 1897, 1898, 1899, 1900, 1901, 1902, 1903, 1904, 1905, 1906, 1907, 1908, 1909, 1910, 1911, 1912, 1913, 1914, 1915, 1916, 1917, 1918, 1919, 1920, 1921, 1922, 1923, 1924, 1925, 1926, 1927, 1928, 1929, 1930, 1931, 1932, 1933, 1934, 1935, 1936, 1937, 1938, 1939, 1940, 1941, 1942, 1943, 1944, 1945, 1946, 1947, 1948, 1949, 1950, 1951, 1952, 1953, 1954, 1955, 1956, 1957, 1958, 1959, 1960, 1961, 1962, 1963, 1964, 1965, 1966, 1967, 1968, 1969, 1970, 1971, 1972, 1973, 1974, 1975, 1976, 1977, 1978, 1979, 1980, 1981, 1982, 1983, 1984, 1985, 1986, 1987, 1988, 1989, 1990, 1991, 1992, 1993, 1994, 1995, 1996, 1997, 1998, 1999, or 2000 or more contiguous amino acids or nucleic acids, or any range derivable therein, of SEQ ID NOS: 1-17 or of a nucleic acid that is complementary to one of SEQ ID NOS:1 and 3-17, or of a nucleic acid that encodes an protein with an amino acid sequence with at least 85%, 86%, 87%, 88%, 89%, 90%, 91%, 92%, 93%, 94%, 95%, 96%, 97%, 98%, 99%, or 100% sequence identity to SEQ ID NO:2.

In some embodiments, the polypeptide comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOS:1-17 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS:1-17 or of a nucleic acid that is complementary to one of SEQ ID NOS:1 and 3-17.

The polypeptides or polynucleotides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600 substitutions.

The substitution may be at amino acid position or nucleic acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of one of SEQ ID NOS:1-17 or of a nucleic acid that is complementary to one of SEQ ID NOS:1 and 3-17.

The polypeptides or polynucleotides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 or more amino acids or nucleic acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide (e.g., wild-type BRD9 or a functional equivalent thereof) may be employed. The generation of recombinant expression vectors, and the elements included therein, are discussed herein. Alternatively, the protein to be produced may be an endogenous protein normally synthesized by the cell used for protein production.

For example, in some embodiments, the disclosure provides isolated BRD9 protein, or an isolated variant thereof, with one or more mutations thereof, as defined in more detail above. In some embodiments, the variant has one or more conservative mutations (i.e., substitutions) such that there is substantial functional equivalency in facilitating ncBAF formation. In some embodiments, the isolated BRD9 protein or functional equivalent thereof has at least about 85% (e.g., 86%, 87%, 88%, 89%, 90%, 91%, 92%, 93%, 94%, 95%, 96%, 97%, 98%, 99%, or 100%) sequence identity to SEQ ID NO:2.

In other embodiments, the disclosure provides nucleic acids encoding a protein with about 85% (e.g., 86%, 87%, 88%, 89%, 90%, 91%, 92%, 93%, 94%, 95%, 96%, 97%, 98%, 99%, or 100%) sequence identity to SEQ ID NO:2. For example, an illustrative, non-limiting example of a cDNA sequence encoding a human wild-type BRD9 protein. In some embodiments, the nucleic acid is integrated into a vector. The vector can be, e.g., an expression vector, that is configured to facilitate expression in a target cell. Expression vectors typically have one or more promoter and/or enhancer elements that are operatively linked to the incorporated nucleic acid that encodes the protein that is intended to be expressed.

Methods

In another aspect, the disclosure provides a method of increasing functional expression of BRD9 in a cell, comprising contacting the cell with an effective amount of a BRD9 activating agent. The term “functional expression” refers to transcription of the BRD9 gene to create a messenger RNA that does not include the poison exon, but instead encodes a wild-type BRD9 protein (e.g., with a sequence corresponding to SEQ ID NO:2) that is a functional component of the non-canonical BAF, such that expression promotes formation of the ncBAF complex.

The cell can be a transformed cell, i.e., a cell that has undergone a genetic or phenotypic change that alters its growth rate compared to normal cells of the same type. In some embodiments, the cell is a cancer cell selected from a blood cancer, bladder cancer, uveal melanoma, cutaneous cancer, pancreatic cancer, breast cancer, prostate cancer, genitourinary cancer, myeloid cancer, and lymphoid cancer.

In some embodiments, the cell comprises one or more SF3B1 mutations. Exemplary, non-limiting SF3B1 mutations comprise E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, and M971V. The cell can comprise one or more of such exemplary mutations and the like.

The activating agent can be any of the activating agents described herein. In some embodiments, the BRD9 activating agent comprises a BRD9 polypeptide or nucleic acid encoding a BRD9 polypeptide. For example, the nucleic acid encoding the BRD polypeptide can be incorporated into an appropriate expression vector, as described in more detail below.

In some embodiments, the BRD9 activating agent comprises a comprises a splicing modifier. The splicing modifier can be an antisense nucleic acid having base complementarity with a BRD9 splice site, as described herein. For example, the splicing modifier can bind to the branch point, 5′ splice site, or 3′ splice site of the poison exon, exon 14a. In other embodiments, the splicing modifier can mutate the branch point, 5′ splice site, or 3′ splice site of the poison exon, exon 14a. Nucleic acid splicing modifiers can comprise modified nucleic acids, as described herein. In some embodiments, the splicing modifier comprises a morpholino. In other embodiments, the splicing modifier is a non-nucleic acid agent that modifies, e.g., prevents, aberrant splicing, e.g., aberrant inclusion of the poison exon, into the BRD9 transcript. the splicing modifier comprises a non-specific inhibitor of splice site recognition sites. Exemplary, non-limiting non-nucleic acid agents encompassed by the disclosure as splicing modifiers include pladienolides (A-G, described in Sakai T, et al. Pladienolides, new substances from culture of Streptomyces platensis Mer-11107. I. Taxonomy, fermentation, isolation and screening. J Antibiot (Tokyo) 2004; 57:173-179, which is herein incorporated by reference), Herboxiedene (GEX1A and other GEX family members), 6-norherboxidiene, FR901463, FR901464, FR901465, Meayamycin, Spliceostatin A, (methylated derivative of FR901464), TG003, SRPIN340, Cpd-1 polypeptide, Cpd-2 polypeptide, and Cpd-391 polypeptide.

In some embodiments, the method is performed in vitro in a cell culture. Such embodiments include ex vivo assays wherein a cell derived from a subject (e.g., via biopsy) is administered a splicing modifier to ascertain the effects on the cell.

In some embodiments, the method is performed in vivo in a subject with cancer, a pre-malignant disease, or a dysplastic disease, as defined herein. Accordingly, the method can be characterized as a method for treating cancer, a pre-malignant disease, or a dysplastic disease in a subject.

As used herein, the terms “treatment,” “treating,” “therapeutic intervention,” and the like, refer to administering the BRD activating agent or therapy, or other agent for increasing non-canonical BAF formation, for the purpose of obtaining an effect. The effect can be prophylactic in terms of completely or partially preventing a disease or symptom thereof and/or can be therapeutic in terms of achieving a partial or complete cure for a disease and/or symptoms of the disease. “Treatment,” as used herein, can include treatment of a tumor in a mammal, particularly in a human, and includes inhibiting the disease, i.e., arresting or slowing its development, preventing recurrence of the disease, and/or relieving the disease, i.e., causing regression of the disease.

The term “subject” as used above in reference to the methods can refer to any animal with the target cell type of interest. Subjects are typically mammals and can include the non-limiting examples of primates (including, e.g., human, monkey, and the like), rodent (including, e.g., rat, mouse, guinea pig, and the like), dog, cat, horse, cow, pig, sheep, and the like. In some specific embodiments, the subject is a human.

The method can comprise administering a BRD9 activating therapy to the subject. The method comprises increasing non-canonical BAF formation in the subject.

In some embodiments, the subject has been diagnosed with cancer, a pre-malignant disease, or a dysplastic disease, as described herein. The cancer or disease can be characterized as a SF3B1-mutant cancer or disease. For example, the subject can be determined to have one or more mutations in the gene encoding SF3B1 in one or more cells in the body. Exemplary mutations include, but are not limited to the SF3B1 mutation comprising E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, and M971V.

In some embodiments, the subject has and/or has been diagnosed with a cancer or disease selected from, e.g., blood cancer, bladder cancer, uveal melanoma, cutaneous cancer, pancreatic cancer, breast cancer, prostate cancer, genitourinary cancer, myeloid cancer, lymphoid cancer, myelodysplastic syndrome, pre-malignant myeloid disease, and the like.

Embodiments of the treatment include administering a BRD9 activating therapy comprising a BRD9 polypeptide or nucleic acid encoding a BRD9 polypeptide, as described herein. In some embodiments, the methods include administration of a RNA encoding a BRD9 polypeptide to a subject. Other embodiments include administering an agent that activates transcription of the endogenous BRD9 gene. The BRD activating therapy can comprise splicing modifiers, as described herein, such as antisense nucleic acid having base complementarity with a BRD9 splice site, for example an antisense molecule that binds to the branch point, 5′ splice site, or 3′ splice site of the poison exon, exon 14a. In some embodiments, the splicing modifier mutates the branch point, 5′ splice site, or 3′ splice site of the poison exon, exon 14a, or the exonic splicing enhancer of the poison exon, exon 14a. Such embodiments include therapies comprising, e.g., CRISPR/Cas9 reagents that implement mutations in the target nucleic acid that functionally prevent recognition of the alternative splice site by mutant SF3B1. Techniques pertaining to the transfer of nucleic acids into cells are well-known to those of ordinary skill in the art. Exemplary techniques are discussed below.

In other embodiments, the splicing modifier is a non-nucleic acid agent that modifies, e.g., prevents, aberrant splicing, e.g., aberrant inclusion of the poison exon, into the BRD9 transcript, as described in more detail above.

In some embodiments, the subject has previously been treated for the disorder or disease. In some embodiments, the subject has been determined to be non-responsive to the previous therapy. For example, the cancer or disorder being addressed can be classified as refractory or recurrent.

In some embodiments, the treatment is a combination treatment that further comprises administration of an additional therapy.

Modes of administration, e.g., systemic, e.g., intravenous administration, are described herein.

The administration of a therapeutic amount encompasses sufficient quantity, or a sufficient number of administrations, such that a desired therapeutic effect is achieved. See, e.g., the definition of “treat” above. In some embodiments, the BRD9 activating therapy stabilizes or increases ncBAF formation in the target cells.

Various components of the BRD9 activating therapies are described in more detail below.

A. Viral Vectors

In certain embodiments, transfer of an expression construct into a cell is accomplished using a viral vector. Techniques using “viral vectors” are well-known in the art. A viral vector is meant to include those constructs containing viral sequences sufficient to (a) support packaging of the expression cassette and (b) to ultimately express a recombinant gene construct that has been cloned therein.

In particular embodiments, the viral vector is a lentivirus vector. Lentivirus vectors have been successfully used in infecting stem cells and providing long term expression.

Another method for delivery of a nucleic acid involves the use of an adenovirus vector. Adenovirus vectors are known to have a low capacity for integration into genomic DNA. Adenovirus vectors result in highly efficient gene transfer.

Adenoviruses are currently the most commonly used vector for gene transfer in clinical settings. Among the advantages of these viruses is that they are efficient at gene delivery to both nondividing and dividing cells and can be produced in large quantities. The vector comprises a genetically engineered form of adenovirus (Grunhaus et al, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. A person of ordinary skill in the art would be familiar with experimental methods using adenoviral vectors.

The adenovirus vector may be replication defective, or at least conditionally defective, and the nature of the adenovirus vector is not believed to be crucial to the successful practice of the invention. The adenovirus may be of any of the 42 different known serotypes or subgroups A-F and other serotypes or subgroups are envisioned. Adenovirus type 5 of subgroup C is the starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector. Adenovirus growth and manipulation is known to those of skill in the art, and exhibits broad host range in vitro and in vivo. Modified viruses, such as adenoviruses with alteration of the CAR domain, may also be used. Methods for enhancing delivery or evading an immune response, such as liposome encapsulation of the virus, are also envisioned. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains two long terminal repeat (LTR) sequences present at the 5′ and 3′ ends of the viral genome. These contain strong promoter and enhancer sequences and are also required for integration in the host cell genome (Coffin, 1990).

In order to construct a retroviral vector, a nucleic acid encoding a nucleic acid or gene of interest is inserted into the viral genome in the place of certain viral sequences to produce a virus that is replication-defective. A person of ordinary skill in the art would be familiar with well-known techniques that are available to construct a retroviral vector.

Adeno-associated virus (AAV) is an attractive vector system for use in the present invention as it has a high frequency of integration and it can infect nondividing cells, thus making it useful for delivery of genes into mammalian cells in tissue culture (Muzyczka, 1992). AAV has a broad host range for infectivity (Tratschin et al., 1984; Laughlin et al, 1986; Lebkowski et al, 1988; McLaughlin et al, 1988), which means it is applicable for use with the present invention. Details concerning the generation and use of rAAV vectors are described in U.S. Pat. Nos. 5,139,941 and 4,797,368, each incorporated herein by reference.

Typically, recombinant AAV (rAAV) virus is made by cotransfecting a plasmid containing the gene of interest flanked by the two AAV terminal repeats (McLaughlin et al, 1988; Samulski et al, 1989; each incorporated herein by reference) and an expression plasmid containing the wild-type AAV coding sequences without the terminal repeats, for example pIM45 (McCarty et al., 1991; incorporated herein by reference). A person of ordinary skill in the art would be familiar with techniques available to generate vectors using AAV virus.

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995). A person of ordinary skill in the art would be familiar with well-known techniques for use of HSV as vectors.

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome.

Other viral vectors may be employed as constructs in the present invention. For example, vectors derived from viruses such as poxvirus may be employed. A molecularly cloned strain of Venezuelan equine encephalitis (VEE) virus has been genetically refined as a replication competent vaccine vector for the expression of heterologous viral proteins (Davis et al., 1996). Studies have demonstrated that VEE infection stimulates potent CTL responses and it has been suggested that VEE may be an extremely useful vector for immunizations (Caley et al., 1997). It is contemplated in the present invention, that VEE virus may be useful in targeting dendritic cells.

A polynucleotide may be housed within a viral vector that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

Nonviral Gene Transfer

Several non-viral methods for the transfer of nucleic acids into cells also are contemplated by certain aspects of the present invention. These include calcium phosphate precipitation (Graham and Van Der Eb, 1973; Chen and Okayama, 1987; Rippe et al, 1990) DEAE-dextran (Gopal, 1985), electroporation (Tur-Kaspa et al, 1986; Potter et al, 1984), nucleofection (Trompeter et al, 2003), direct microinjection (Harland and Weintraub, 1985), DNA-loaded liposomes (Nicolau and Sene, 1982; Fraley et al, 1979) and lipofectamine-DNA complexes, polyamino acids, cell sonication (Fechheimer et al, 1987), gene bombardment using high velocity microprojectiles (Yang et al, 1990), polycations (Boussif et al, 1995) and receptor-mediated transfection (Wu and Wu, 1987; Wu and Wu, 1988). Some of these techniques may be successfully adapted for in vivo or ex vivo use. A person of ordinary skill in the art would be familiar with the techniques pertaining to use of nonviral vectors, and would understand that other types of nonviral vectors than those disclosed herein are contemplated by the present invention. In a further embodiment of the invention, the expression cassette may be entrapped in a liposome or lipid formulation. Liposomes are vesicular structures characterized by a phospholipid bilayer membrane and an inner aqueous medium. Multilamellar liposomes have multiple lipid layers separated by aqueous medium. Also contemplated is a gene construct complexed with Lipofectamine (Gibco BRL). One of ordinary skill in the art would be familiar with techniques utilizing liposomes and lipid formulations.

Lipid-Based Nanovesicles

In some embodiments, a lipid-based nanovesicle, such as a liposome, an exosome, lipid preparations, lipid-based vesicles (e.g., a DOTAP:cholesterol vesicle) are employed in the methods of the disclosure. In some embodiments, the nanovesicle comprising a TERT polypeptide or nucleic acid encoding a TERT polypeptide is administered to the subject. Lipid-based nanovesicles may be positively charged, negatively charged or neutral.

1. Liposomes

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes, and multivesicular liposomes. Liposomes provided herein may be positively charged, negatively charged, or neutrally charged. In certain embodiments, the liposomes are neutral in charge.

A multilamellar liposome has multiple lipid layers separated by aqueous medium. Such liposomes form spontaneously when lipids comprising phospholipids are suspended in an excess of aqueous solution. The lipid components undergo self-rearrangement before the formation of closed structures and entrap water and dissolved solutes between the lipid bilayers. Lipophilic molecules or molecules with lipophilic regions may also dissolve in or associate with the lipid bilayer.

In some embodiments, a polypeptide, a nucleic acid, or a small molecule drug may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

A liposome used according to the present embodiments can be made by different methods, as would be known to one of ordinary skill in the art. For example, a phospholipid, such as for example the neutral phospholipid dioleoylphosphatidylcholine (DOPC), is dissolved in tert-butanol. The lipid(s) is then mixed with a polypeptide, nucleic acid, and/or other component(s). Tween 20 is added to the lipid mixture such that Tween 20 is about 5% of the composition's weight. Excess tert-butanol is added to this mixture such that the volume of tert-butanol is at least 95%. The mixture is vortexed, frozen in a dry ice/acetone bath and lyophilized overnight. The lyophilized preparation is stored at −20° and can be used up to three months. When required the lyophilized liposomes are reconstituted in 0.9% saline.

Alternatively, a liposome can be prepared by mixing lipids in a solvent in a container, e.g., a glass, pear-shaped flask. The container should have a volume ten-times greater than the volume of the expected suspension of liposomes. Using a rotary evaporator, the solvent is removed at approximately 40° C. under negative pressure. The solvent normally is removed within about 5 min to 2 h, depending on the desired volume of the liposomes. The composition can be dried further in a desiccator under vacuum. The dried lipids generally are discarded after about 1 week because of a tendency to deteriorate with time.

Dried lipids can be hydrated at approximately 25-50 mM phospholipid in sterile, pyrogen-free water by shaking until all the lipid film is resuspended. The aqueous liposomes can be then separated into aliquots, each placed in a vial, lyophilized and sealed under vacuum.

The dried lipids or lyophilized liposomes prepared as described above may be dehydrated and reconstituted in a solution of a protein or peptide and diluted to an appropriate concentration with a suitable solvent, e.g., DPBS. The mixture is then vigorously shaken in a vortex mixer. Unencapsulated additional materials, such as agents including but not limited to hormones, drugs, nucleic acid constructs and the like, are removed by centrifugation at 29,000×g and the liposomal pellets washed. The washed liposomes are resuspended at an appropriate total phospholipid concentration, e.g., about 50-200 mM. The amount of additional material or active agent encapsulated can be determined in accordance with standard methods. After determination of the amount of additional material or active agent encapsulated in the liposome preparation, the liposomes may be diluted to appropriate concentrations and stored at 4° C. until use. A pharmaceutical composition comprising the liposomes will usually include a sterile, pharmaceutically acceptable carrier or diluent, such as water or saline solution.

Additional liposomes which may be useful with the embodiments of the disclosure include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040, each incorporated herein by reference.

In certain embodiments, the lipid based nanovesicle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanovesicles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes. Phospholipids may be from natural or synthetic sources.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used Phospholipids include glycerophospholipids and certain sphingolipids.

Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

Exosomes

The terms “nanovesicle” and “exosomes,” as used herein, refer to a membranous particle having a diameter (or largest dimension where the particles is not spheroid) of between about 10 nm to about 1000 nm, more typically between 30 nm and 1000 nm, and most typically between about 50 nm and 750 nm, wherein at least part of the membrane of the exosomes is directly obtained from a cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One preferred method is differential centrifugation from body fluids or cell culture supernatants. Alternatively, exosomes may also be isolated via flow cytometry.

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

Isolation of exosomes based on size, using alternatives to the ultracentrifugation routes, is another option. Successful purification of exosomes using ultrafiltration procedures that are less time consuming than ultracentrifugation, and do not require use of special equipment have been reported. Similarly, a commercial kit is available (EXOMIR™ Bioo Scientific) which allows removal of cells, platelets, and cellular debris on one microfilter and capturing of vesicles bigger than 30 nm on a second microfilter using positive pressure to drive the fluid. However, for this process, the exosomes are not recovered, their RNA content is directly extracted from the material caught on the second microfilter, which can then be used for PCR analysis. HPLC-based protocols could potentially allow one to obtain highly pure exosomes, though these processes require dedicated equipment and are difficult to scale up. A significant problem is that both blood and cell culture media contain large numbers of nanoparticles (some non-vesicular) in the same size range as exosomes. For example, some miRNAs may be contained within extracellular protein complexes rather than exosomes; however, treatment with protease (e.g., proteinase K) can be performed to eliminate any possible contamination with “extraexosomal” protein.

Additional Therapies

The disclosure encompasses combination therapies, i.e., therapies that further comprise administration of an additional therapy for the cancer, pre-malignant disease, and/or dysplastic disease. In some embodiments, the additional therapy comprises an immunotherapy, oncolytic virus, polysaccharide, neoantigen, chemotherapy, radiotherapy, surgery, or other therapy described below or throughout the disclosure.

A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

1. Checkpoint Inhibitors and Combination Treatment

Embodiments of the disclosure may include administration of immune checkpoint inhibitors (also referred to as checkpoint inhibitor therapy), which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art-recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

6. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death. [60]

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

7. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy.

8. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

9. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

10. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

11. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112,113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

12. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

13. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

Nucleic Acids and Oligonucleotides

In certain embodiments, there are recombinant nucleic acids encoding the polypeptides described herein.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or fewer in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein (see above). The term “polynucleotide” may also be used interchangeably with “oligonucleotide” and refer to an antisense oligonucleotide of the disclosure. Accordingly, modifications to polynucleotides described herein are applicable embodiments relating to antisense oligonucleotides.

In particular embodiments, there are isolated nucleic acid segments and recombinant vectors incorporating nucleic acid sequences that encode a polypeptides (e.g., a polymerase, RNA polymerase, one or more truncated polymerase domains or interaction components that are polypeptides) that drive gene transcription dependent on polymerase activity from the polymerase domains when the interaction components interact. The term “recombinant” may be used in conjunction with a polypeptide or the name of a specific polypeptide, and this generally refers to a polypeptide produced from a nucleic acid molecule that has been manipulated in vitro or that is a replication product of such a molecule.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

A. Vectors

Polypeptides may be encoded by a nucleic acid molecule. The nucleic acid molecule can be in the form of a nucleic acid vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Vectors may be used in a host cell to produce a polymerase, RNA polymerase, one or more truncated polymerase domains or interaction components that are fused, attached or linked to the one or more truncated RNA polymerase domains.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described herein.

Cells

The disclosure provides methods for modifying a target RNA of interest, in particular in prokaryotic cells, eukaryotic cells, tissues, organs, or organisms, more in particular in mammalian cells, tissues, organs, or organisms. The target RNA may be comprised in a nucleic acid molecule within a cell. In some embodiments, the target RNA is in a eukaryotic cell, such as a mammalian cell or a plant cell. The mammalian cell may be a human, non-human primate, bovine, porcine, rodent or mouse cell. The cell may be a non-mammalian eukaryotic cell such as poultry, fish or shrimp. The plant cell may be of a crop plant such as cassava, corn, sorghum, wheat, or rice. The plant cell may also be of an algae, tree or vegetable. The modulation of the RNA induced in the cell by the methods, systems, and compositions of the disclosure may be such that the cell and progeny of the cell are altered for improved production of biologic products such as an antibody, starch, alcohol or other desired cellular output. The modulation of the RNA induced in the cell may be such that the cell and progeny of the cell include an alteration that changes the biologic product produced.

The mammalian cell may be a human or non-human mammal, e.g., primate, bovine, ovine, porcine, canine, rodent, Leporidae such as monkey, cow, sheep, pig, dog, rabbit, rat or mouse cell. The cell may be a non-mammalian eukaryotic cell such as poultry bird (e.g., chicken), vertebrate fish (e.g., salmon) or shellfish (e.g., oyster, clam, lobster, shrimp) cell.

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

Expression Systems

Numerous expression systems exist that comprise at least a part or all of the compositions discussed above. Prokaryote- and/or eukaryote-based systems can be employed for use with an embodiment to produce nucleic acid sequences, or their cognate polypeptides, proteins and peptides. For example, the vectors, fusion proteins, RNA hairpin binding proteins, RNA targeting molecules, RNA regulatory domain, and accessory proteins of the disclosure may utilize an expression system, such as an inducible or constitutive expression system. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems, other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Antisense Oligonucleotides

“Oligonucleotide” refers to a molecule comprising short fragments of nucleic acid polymers, such as DNA or RNA. The individual nucleotide subunits of the DNA or RNA include natural canonical nucleotides, such as deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T) for DNA and A, uridine 5′-triphosphate (UTP or U), G, and C for RNA, and can also include analogs of natural nucleotides, such as labeled nucleotides. Nucleotides include those nucleotides containing modified bases, modified sugar moieties, and modified phosphate backbones, for example as described in U.S. Pat. No. 5,866,336 to Nazarenko et al. Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxy acetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others. Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof. The individual nucleotides subunits in typical oligonucleotide are joined by a phospho-diester linkage between the sugar of one nucleotide and the phosphate of the next, resulting in an alternating sugar-phosphate backbone.

In some embodiments, the oligonucleotide may be a modified oligonucleotide that has non-naturally occurring portions. Modified oligonucleotide can have altered sugar moieties, altered base moieties, as described above, or altered inter-sugar linkages. The term “oligomers” is intended to encompass oligonucleotides, oligonucleotide analogs or oligonucleosides. Thus, in speaking of “oligomers” reference is made to a series of nucleosides or nucleoside analogs that are joined via either natural phosphodiester bonds or other linkages, including the four atom linkers. Although the linkage generally is from the 3′ carbon of one nucleoside to the 5′ carbon of a second nucleoside, the term “oligomer” can also include other linkages such as 2′-5′ linkages.

Modified oligonucleotides can include modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such as a substitution for the inter-sugar phosphodiester linkage are made, the resulting material is no longer a true nucleic acid species. All such compounds are considered to be analogs.

The modified oligonucleotides may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound. Preferred linkages have structure CH₂—RA-NR₁CH₂, CH₂—NR₁—RA-CH₂, RA-NR₁—CH₂—CH₂, CH₂—CH₂—NR₁—RA, CH₂—CH₂—RA-NR₁, or NR₁—RA-CH₂—CH₂ where RA is O or NR₂.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Antisense oligonucleotides of the disclosure may be at least or at most 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides (or any derivable range therein). In some embodiments, oligonucleotides of the disclosure include a flanking sequence. Several types of flanking sequences may be used. In some embodiments, flanking sequences are used to modify the binding of a protein to said molecule or oligonucleotide, or to modify a thermodynamic property of the oligonucleotide, or to modify target RNA binding affinity.

In some embodiments, an oligonucleotide of the disclosure is capable of interfering with the inclusion of exon 14a of the alternatively spliced BRD9 pre-mRNA. In some embodiments, the oligonucleotide comprises a nucleotide-based or nucleotide or an antisense oligonucleotide sequence of between 21 and 50 nucleotides or bases, between 21 and 40 nucleotides, between 21 and 30 nucleotides, such as 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 31 nucleotides, 32 nucleotides, 33 nucleotides, 34 nucleotides, 35 nucleotides, 36 nucleotides, 37 nucleotides, 38 nucleotides, 39 nucleotides, 40 nucleotides, 41 nucleotides, 42 nucleotides, 43 nucleotides, 44 nucleotides, 45 nucleotides, 46 nucleotides, 47 nucleotides, 48 nucleotides, 49 nucleotides or 50 nucleotides.

A nucleotide sequence of an oligonucleotide of the disclosure may contain a RNA residue, a DNA residue, a nucleotide analogue or equivalent as will be further detailed herein. In some embodiments, the oligonucleotide comprises at least one residue comprising a modified base, and/or a modified backbone, and/or a non-natural internucleoside linkage, or a combination of these modifications.

In some embodiments, the oligonucleotide comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

In some embodiments, the modified oligonucleotide comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993) Nature 365, 566-568).

In some embodiments, the modified oligonucleotide comprises a morpholino nucleotide analog or equivalent, in which the ribose or deoxyribose sugar is replaced by a 6-membered morpholino ring. In some embodiments, the modified oligonucleotide comprises phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In some embodiments, the modified oligonucleotide comprises a substitution of at least one of the non-bridging oxygens in the phosphodiester linkage. This modification slightly destabilizes base-pairing but adds significant resistance to nuclease degradation. In some embodiments, the modified oligonucleotide comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

In some embodiments, the modified oligonucleotide comprises one or more sugar moieties that are mono- or disubstituted at the 2′, 3′ and/or 5′ position such as a —OH; —F; substituted or unsubstituted, linear or branched lower (C1-C10) alkyl, alkenyl, alkynyl, alkaryl, allyl, aryl, or aralkyl, that may be interrupted by one or more heteroatoms; O-, S-, or N-alkyl; O-, S-, or N-alkenyl; O-, S- or N-alkynyl; O-, S-, or N-allyl; O-alkyl-O-alkyl, -methoxy, -aminopropoxy; aminoxy, methoxyethoxy; -dimethylaminooxyethoxy; and -dimethylaminoethoxyethoxy. The sugar moiety can be a pyranose or derivative thereof, or a deoxypyranose or derivative thereof, preferably a ribose or a derivative thereof, or deoxyribose or derivative thereof. In some embodiments, the modified oligonucleotide comprises Locked Nucleic Acid (LNA), in which the 2′-carbon atom is linked to the 3′ or 4′ carbon atom of the sugar ring thereby forming a bicyclic sugar moiety. In some embodiments, the LNA comprises 2′-0,4′-C-ethylene-bridged nucleic acid (Morita et al. 2001. Nucleic Acid Res Supplement No. 1: 241-242).

It is understood by a skilled person that it is not necessary for all positions in an antisense oligonucleotide to be modified uniformly. In addition, more than one of the aforementioned analogues or equivalents may be incorporated in a single antisense oligonucleotide or even at a single position within an antisense oligonucleotide. In certain embodiments, an antisense oligonucleotide of the invention has at least two different types of analogues or equivalents.

In some embodiments, the modified oligonucleotide comprises a 2′-O-alkyl phosphorothioate antisense oligonucleotide, such as 2′-O-methyl modified ribose (RNA), 2′-O-ethyl modified ribose, 2′-O-propyl modified ribose, and/or substituted derivatives of these modifications such as halogenated derivatives. In some embodiments, the modified oligonucleotide comprises a 2′-O-methyl phosphorothioate ribose.

A functional equivalent of a molecule of the disclosure may be defined as an oligonucleotide as defined herein wherein an activity of said functional equivalent is retained to at least some extent. Preferably, an activity of said functional equivalent is inducing exon 14a skipping and providing a functional and/or non-tumorigenic BRD9 protein. Said activity of said functional equivalent is therefore preferably assessed by detection of exon 14a skipping and/or quantifying the amount of a functional BRD9 protein or ncBAF complex.

An antisense oligonucleotide can be linked to a moiety that enhances uptake of the antisense oligonucleotide in cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain. In some embodiments, the oligonucleotide comprises a peptide-linked PMO.

Methods for Modifying Genomic DNA

In certain embodiments, the genomic DNA is modified either to include additional mutations, insertions, or deletions, or to integrate certain nucleic acids of the disclosure, such as nucleic acids that encode for a BRD9 polypeptide or nucleic acids comprising antisense embodiments of the disclosure. In some embodiments, a nucleic acid encoding a polypeptide of the disclosure is integrated into the genomic DNA of a cell. In some embodiments, the integration is targeted integration. In some embodiments, genomic nucleic acids are modified. For example, splice sites, such as the 3′ or 5′ splice site, the branch point, or the exonic enhancer of the poison exon, exon 14a may be mutated so that the splicing of exon 14a is reduced, inhibited, or abolished in a cell or a subject. In some embodiments, targeted integration or genomic DNA modification is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids.

Methods for modifying the genomic DNA may be achieved through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, DC31 integrase, Bxbl-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Modification of the genomic DNA may also include targeted endonucleases such as zinc finger nucleases (ZFNs), meganucleases, transcription activator-like effector nucleases (TALENs), CRISPR/Cas-like endonucleases, I-Tevl nucleases or related monomeric hybrids, or artificial targeted DNA double strand break inducing agents. Exemplary targeting endonucleases are further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Multiple recognition sequences may be present in a single landing pad, allowing the landing pad to be targeted sequentially by two or more polynucleotide modification enzymes such that two or more unique nucleic acids (comprising, among other things, receptor genes and/or inducible reporters) can be inserted. Alternatively, the presence of multiple recognition sequences in the landing pad, allows multiple copies of the same nucleic acid to be inserted into the landing pad. When two nucleic acids are targeted to a single landing pad, the landing pad includes a first recognition sequence for a first polynucleotide modification enzyme (such as a first ZFN pair), and a second recognition sequence for a second polynucleotide modification enzyme (such as a second ZFN pair). Alternatively, or additionally, individual landing pads comprising one or more recognition sequences may be integrated at multiple locations. Increased protein expression may be observed in cells transformed with multiple copies of a payload. Alternatively, multiple gene products may be expressed simultaneously when multiple unique nucleic acid sequences comprising different expression cassettes are inserted, whether in the same or a different landing pad. Regardless of the number and type of nucleic acid, when the targeting endonuclease is a ZFN, exemplary ZFN pairs include hSIRT, hRSK4, and hAAVS1, with accompanying recognition sequences.

Generally speaking, a landing pad used to facilitate targeted integration may comprise at least one recognition sequence. For example, a landing pad may comprise at least one, at least two, at least three, at least four, at least five, at least six, at least seven, at least eight, at least nine, or at least ten or more recognition sequences. In embodiments comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable or screenable genes as described herein, such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used to modify genomic DNA is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some embodiments, targeted integrated can be achieved through the use of an integrase. For example, the phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been shown to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one embodiment, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals.

DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

Administration of Therapeutic Compositions

The therapy provided herein may comprise administration of a combination of therapeutic agents, such as a first BRD9 activating therapy and a second therapy. The therapies may be administered in any suitable manner known in the art. For example, the first and second cancer treatment may be administered sequentially (at different times) or concurrently (at the same time). In some embodiments, the first and second therapies are administered in a separate composition. In some embodiments, the first and second therapies are in the same composition. In some embodiments, methods and compositions of the disclosure comprise administration of an additional therapy. In some embodiments, the additional therapy comprises a cancer therapy such as an immunotherapy, a chemotherapy, radiation, or surgery.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed, for example, a first cancer treatment is “A” and a second cancer treatment is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

The therapies comprising therapeutic agents such as polypeptides, nucleic acids, additional therapies, or BRD9 activating therapies of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, intratumoral, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 nM to 1 μM. For example, in some embodiments, the effective dose provides a blood level of about 10 nM to 1 μM; or about 50 nM to 1 μM; or about 75 nM to 1 μM; or about 100 nM to 1 μM; or about 150 nM to 1 μM; or about 200 nM to 1 μM; or about 250 nM to 1 μM; or about 300 nM to 1 μM; or about 350 nM to 1 μM; or about 400 nM to 1 μM; or about 450 nM to 1 μM; or about 500 nM to 1 μM; or about 550 nM to 1 μM; or about 600 nM to 1 μM; or about 650 nM to 1 μM; or about 700 nM to 1 μM; or about 750 nM to 1 μM; or about 800 nM to 1 μM; or about 850 nM to 1 μM; or about 900 nM to 1 μM; or about 950 nM to 1 μM; or about 10 nM to about 250 nM; or about 200 nM to about 400 nM; or about 300 nM to about 500 nM; or about 400 nM to about 600 nM; or about 500 nM to about 700 nM; or about 600 nM to about 800 nM; or about 700 nM to about 900 nM (or any range derivable therein).

In some embodiments, the dose can provide the following blood level of the agent that results from a therapeutic agent being administered to a subject: about, at least about, or at most about 1, 2, 3, 4, 5, 6, 7, 8, 9, 10, 15, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 609, 700, 710, 720, 730, 740, 750, 760, 770, 780, 790, 800, 810, 820, 830, 840, 850, 860, 870, 880, 890, 900, 910, 920, 930, 940, 950, 960, 907, 980, 990, or 1000 nM or any range derivable therein.

In certain embodiments, the effective dose of the pharmaceutical composition is one which can provide a blood level of about 1 μM to 150 μM. In another embodiment, the effective dose provides a blood level of about 1 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

Treatment of Disease

The methods of the disclosure may be used to treat a cancer. The cancers amenable for treatment may include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, recurrent and/or metastatic breast cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

Kits

Certain aspects concern kits containing compositions described herein or compositions to implement methods described herein.

In various aspects, a kit is envisioned containing therapeutic agents and/or other therapeutic and delivery agents. In some embodiments, a kit for preparing and/or administering a therapy described herein may be provided. The kit may comprise one or more sealed vials containing any of the pharmaceutical compositions, therapeutic agents and/or other therapeutic and delivery agents. In some embodiments, the lipid is in one vial, and the therapeutic agent is in a separate vial. The kit may include, for example, at least one BRD9 activating therapy, one or more lipid component, as well as reagents to prepare, formulate, and/or administer the components described herein or perform one or more steps of the methods. In some embodiments, the kit may also comprise a suitable container means, which is a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill. The instruction information may be in a computer readable media containing machine-readable instructions that, when executed using a computer, cause the display of a real or virtual procedure of delivering a pharmaceutically effective amount of a therapeutic agent.

In some embodiments, kits may be provided to evaluate the expression or mutation status of BRD9, SF3B1, or related molecules. Such kits can be prepared from readily available materials and reagents. For example, such kits can comprise any one or more of the following materials: enzymes, reaction tubes, buffers, detergent, primers and probes, nucleic acid amplification, and/or hybridization agents. In a particular embodiment, these kits allow a practitioner to obtain samples in blood, tears, semen, saliva, urine, tissue, serum, stool, colon, rectum, sputum, cerebrospinal fluid and supernatant from cell lysate. In another embodiment, these kits include the needed apparatus for performing RNA extraction, RT-PCR, and gel electrophoresis. Instructions for performing the assays can also be included in the kits.

Kits may comprise components, which may be individually packaged or placed in a container, such as a tube, bottle, vial, syringe, or other suitable container means. The components may include probes, primers, antibodies, arrays, negative and/or positive controls. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

The kit can further comprise reagents for labeling BRD9 or BAF complexes in the sample. The kit may also include labeling reagents, including at least one of amine-modified nucleotide, poly(A) polymerase, and poly(A) polymerase buffer. Labeling reagents can include an amine-reactive dye or any dye known in the art.

The components of the kits may be packaged either in aqueous media or in lyophilized form. The container means of the kits will generally include at least one vial, test tube, flask, bottle, syringe or other container means, into which a component may be placed, and preferably, suitably aliquotted. Where there is more than one component in the kit (labeling reagent and label may be packaged together), the kit also will generally contain a second, third or other additional container into which the additional components may be separately placed. However, various combinations of components may be comprised in a vial. The kits may also include a means for containing the nucleic acids, antibodies or any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred.

Alternatively, the components of the kit may be provided as dried powder(s). When reagents and/or components are provided as a dry powder, the powder can be reconstituted by the addition of a suitable solvent. It is envisioned that the solvent may also be provided in another container means. In some embodiments, labeling dyes are provided as a dried power. It is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 μg or at least or at most those amounts of dried dye are provided in kits in certain aspects. The dye may then be resuspended in any suitable solvent, such as DMSO.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits may include a means for containing the vials in close confinement for commercial sale, such as, e.g., injection and/or blow-molded plastic containers into which the desired vials are retained.

A kit may also include instructions for employing the kit components as well as the use of any other reagent not included in the kit. Instructions may include variations that can be implemented.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, N.Y. (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

Throughout this application, the term “about” is used to indicate that a value includes the inherent variation about or below the reference number. For example, in some embodiments, the term about includes variation of 10%, 9%, 8%, 7%, 6%, 5%, 4%, 3%, 2%, or 1% above and/or below the indicated reference number.

The use of the word “a” or “an” when used in conjunction with the term “comprising” may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.” The phrase “and/or” conceptually means “and” or “or”. To illustrate, A, B, and/or C includes: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Words using the singular or plural number also include the plural and singular number, respectively. Additionally, the words “herein,” “above,” and “below,” and words of similar import, when used in this application, shall refer to this application as a whole and not to any particular portions of the application.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Percent sequence identity” or grammatical equivalents means that a particular sequence has at least a certain percentage of amino acid residues identical to those in a specified reference sequence using an alignment algorithm. An example of an algorithm that is suitable for determining sequence similarity is the BLAST algorithm, which is described in Altschul, et al., J. Mol. Biol. 215:403-410 (1990). Software for performing BLAST analyses is publicly available through the National Center for Biotechnology Information (NCBI) website.

The term “wildtype,” “wild-type,” “WT” and the like refers to a naturally-occurring polypeptide or nucleic acid sequence, i.e., one that does not include a man-made variation.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

This Example describes the investigation into the role of SF3B1 mutations in cancer. Aspects of this example were incorporated into Inoue, D., et al., “Spliceosomal disruption of the non-canonical BAF complex in cancer,” Nature 574:432-436 (2019), which is incorporated herein by reference in its entirety including all supplemental materials and extended data.

### Introduction

SF3B1 is the most commonly mutated RNA splicing factor in cancer, but the mechanisms by which SF3B1 mutations promote malignancy are poorly understood. SF3B1 is subject to recurrent missense mutations at specific residues in myeloid leukaemia and lymphoid leukaemia, as well as in solid tumours, at rates of up to 14-29% for uveal melanoma (UVM) and 65-83% for myelodysplastic syndromes with ring sideroblasts. Consistent with the critical role of SF3B1 in the recognition of 3′ splice sites, several previous studies have reported that SF3B1 mutations induce widespread usage of abnormal 3′ splice sites. Although many mis-spliced genes have been identified in SF3B1-mutant samples, few of these have been functionally implicated in driving disease.

In this study, pan-cancer splicing analyses were integrated with a positive-enrichment CRISPR screen to prioritize splicing alterations that promote tumorigenesis. It is demonstrated that diverse SF3B1 mutations converge on repression of BRD9, which is a core component of the recently described non-canonical BAF chromatin-remodelling complex that also contains GLTSCR1 and GLTSCR1L. Mutant SF3B1 recognizes an aberrant, deep intronic branchpoint within BRD9 and thereby induces the inclusion of a poison exon that is derived from an endogenous retroviral element and subsequent degradation of BRD9 mRNA. Depletion of BRD9 causes the loss of non-canonical BAF at CTCF-associated loci and promotes melanomagenesis. BRD9 is a potent tumour suppressor in uveal melanoma, such that correcting mis-splicing of BRD9 in SF3B1-mutant cells using antisense oligonucleotides or CRISPR-directed mutagenesis suppresses tumour growth. These results implicate the disruption of non-canonical BAF in the diverse cancer types that carry SF3B1 mutations and suggest a mechanism-based therapeutic approach for treating such malignancies.

### Results and Discussion

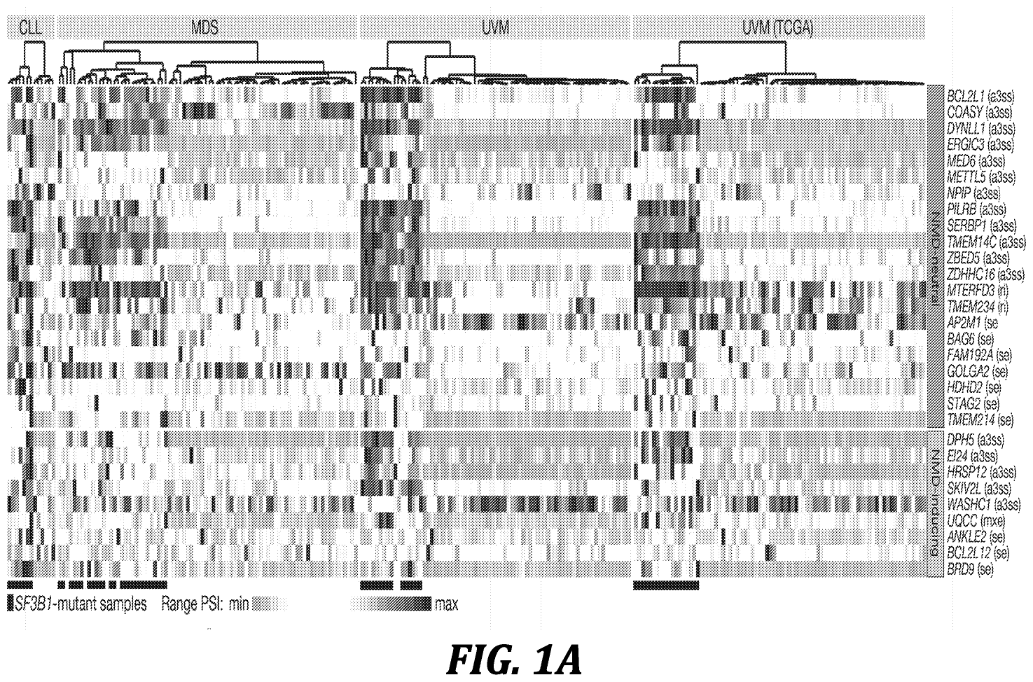

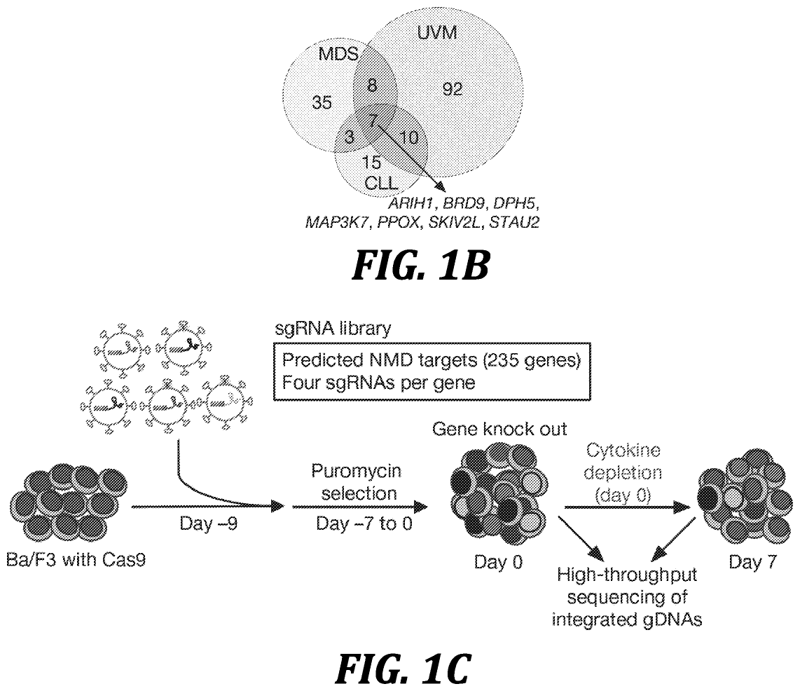

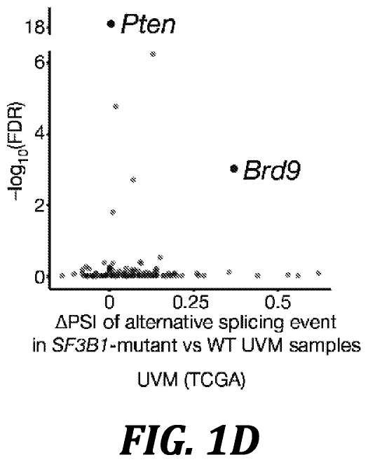

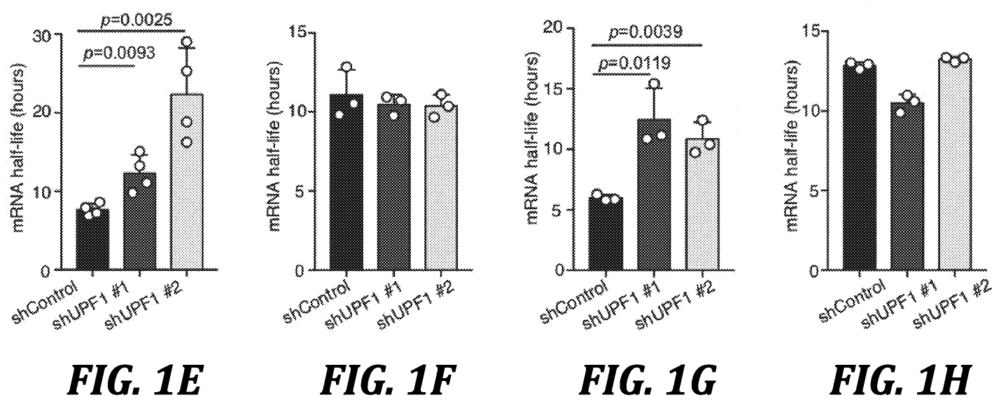

For this investigation, it was hypothesized that effectors of the pro-tumorigenic consequences of SF3B1 mutations might appear as pan-cancer targets of mutant SF3B1. Accordingly, mis-spliced events were identified that were shared between erythroleukaemic (K562) and UVM (MEL270) cells that expressed wild-type SF3B1 or the most-common SF3B1 mutation, SF3B1^{K700E}. A compact set of 40 events exhibited concordant splicing changes, and was sufficient to infer SF3B1 mutational status across 249 samples from patients with chronic lymphocytic leukaemia, myelodysplastic syndromes, and UVM (FIG. 1A).

A single-guide RNA (sgRNA) library was designed that targeted both pan-cancer and cancer-type-specific targets of mutant SF3B1, focusing on genes for which SF3B1 mutations are predicted to cause mis-splicing that triggers nonsense-mediated RNA decay (NMD) (FIG. 1B). It was tested whether the knockout of any of these genes promoted the transformation of Ba/F3 cells (a mouse cell line with a wild-type spliceosome, with a requirement for IL-3 that can be overcome by oncogenic lesions) (FIG. 1C). In addition to the positive control Pten, the screen revealed that the loss of Brd9 promoted the transformation of Ba/F3 cells (FIG. 1D). Brd9 was a notable hit because BRD9 exhibited notable mis-splicing in all cohorts of patients with SF3B1-mutant cancer. Brd9 knockout conferred cytokine independence to mouse 32Dcl3 cells, and growth advantage to human cancer cells with a wild-type spliceosome derived from UVM, cutaneous melanoma, and pancreatic cancer. Briefly, analyses included competition assays to measure the effect of individual sgRNAs on growth of Cas9-expressing Ba/F3 cells, 32Dcl3 cells, MEL270 cells, MEL285 cells 92.1 cells, SK-MEL30 cells, and KPC cells. Cell growth was computed with respect to cells treated with a non-targeting (control) sgRNA and the percentages of GFP+ cells on day 14 were normalized to the percentages on day 2 and heatmaps were generated to correspond to the mean value over n=3 biological replicates. Rpa2 sgRNAs were used as negative control (not shown). By contrast, acute myeloid leukaemia cells (RN2 cells) with rearranged MLL (also known as KMT2A) required BRD9 for growth (not shown), as previously reported.

SF3B1 mutations cause the exonization of a BRD9 intronic sequence, which results in the inclusion of a poison exon that interrupts the open reading frame of BRD9. Phylogenetic analyses indicate the BRD9 poison exon is derived from a primate-specific endogenous retroviral element, explaining its absence from mice. RT-PCR analysis of Brd9 mRNA in murine bone marrow cells, with or without mutated Sf3b1, confirmed lack of the poison exon in the cells (not shown). The inclusion of the poison exon was confirmed to be induced by the expression of endogenous or ectopic mutant SF3B1 in K562 and NALM-6 cells, whereas SF3B1 knockdown in SF3B1 wild-type cells had no effect (briefly, expression was confirmed by RT-PCR and western blot analyses; not shown). The poison exon was included in an SF3B1-mutation-dependent manner in diverse cell lines and in samples of chronic lymphocytic leukaemia, myelodysplastic syndromes and UVM that bear 19 different SF3B1 mutations—but not in healthy tissues. Briefly, RT-PCR was conducted to detect BRD9 with and without the poison exon across (n=2) for K562 cells treated with control shRNA (shRen) or SF3B1-targeting shRNAs; knock-in of the SF3B1K700K, SF3B1^{K700E} or SF3B1^{K666N} mutation into the endogenous locus of SF3B1; overexpression of wild-type SF3B1 or SF3B1^{K700E} cDNA; acute myeloid leukaemia cell lines with wild-type SF3B1 (MV4;11) or a naturally occurring endogenous SF3B1^{K700E} mutation. Additional RT-PCR assays were performed on pancreatic ductal adenocarcinoma cell lines (CFPAC1 and MIA PaCa2, which lack SF3B1 mutations, and Panc05;04 cells which carry SF3B1^{Q699H/K700E}), UVM cell lines (UPMD1 and MEL270 cells lack SF3B1 mutations, and MEL202 and UPMD2 cells which carry SF3B1^{R625G} and SF3B1^{Y623H} mutations, respectively) and a cohort of patients with chronic lymphocytic leukaemia (with SF3B1-WT and SF3B1 with various mutants). RNA-Seq assays were conducted and coverage plots generated of the BRD9 poison exon locus from patient samples with a variety of SF3B1 genotypes and from healthy samples from various tissues (not shown). All SF3B1-mutated samples exhibited BRD9 poison exon inclusion. Comparisons were made to similar tissue samples from healthy subjects (not shown).

The inclusion of the BRD9 poison exon triggered NMD and reduced the half-life of BRD9 mRNA and steady-state levels of full-length BRD9 protein (FIGURES IE-H). Western blot for BRD9 in NALM-6 cells with or without knock-in of an SF3B1 mutation confirmed loss of BRD protein with SF3B1 mutation (not shown). Patients with SF3B1 mutations exhibited reduced total levels of BRD9 mRNA relative to patients with wild-type SF3B1. Briefly, rank plots of BRD9 poison exon inclusion and box plots of gene expression were generated for patients stratified by SF3B1 mutational status (not shown). SF3B1 mutations were strongly associated with high poison exon inclusion and low BRD9 expression. The inventors tested whether the inclusion of the poison exon could result in the production of C-terminally truncated BRD9 by knocking an N-terminal haemagglutinin tag into the BRD9 locus in MEL270 and K562 cells that transgenically express wild-type or mutant SF3B1. Briefly, the single-stranded donor DNA contained a 197-nt fragment, including 83 nt homologous to the BRD9 5′ UTR (upstream of the HA tag) and 87 nt homologous to BRD9 exon 1 (downstream of the start codon). Successful knock-in was confirmed with Sanger sequencing and western blot using anti-BRD9, anti-HA or anti-actin used to probe K562 SF3B1^{K700E} cells carrying an endogenously N-terminally HA-tagged BRD9. Western blot analysis performed for N-terminally haemagglutinin (HA)-tagged endogenous BRD9 in MEL270 cells transduced with empty vector or doxycycline-inducible Flag-SF3B1(WT) or Flag-SF3B1(K700E) confirmed that mutant SF3B1 suppressed levels of full-length BRD9 protein, without generating a truncated BRD9 protein (not shown).

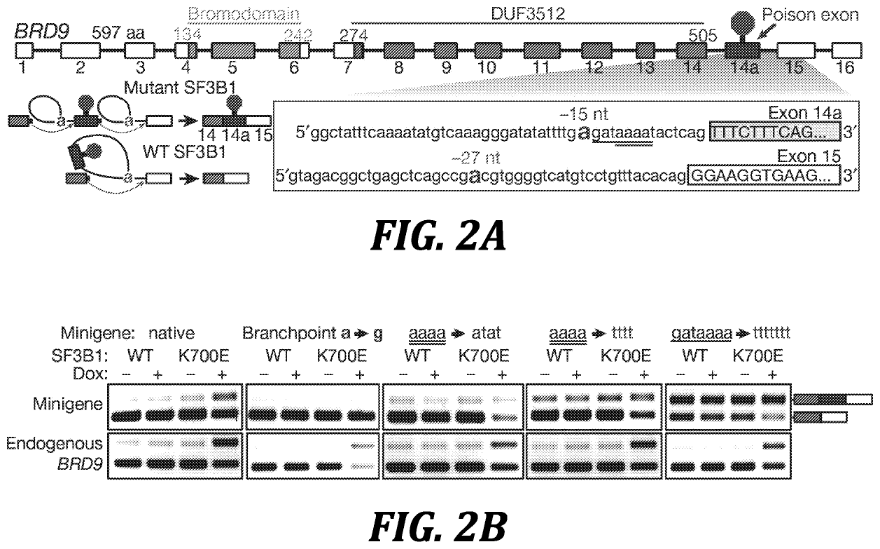

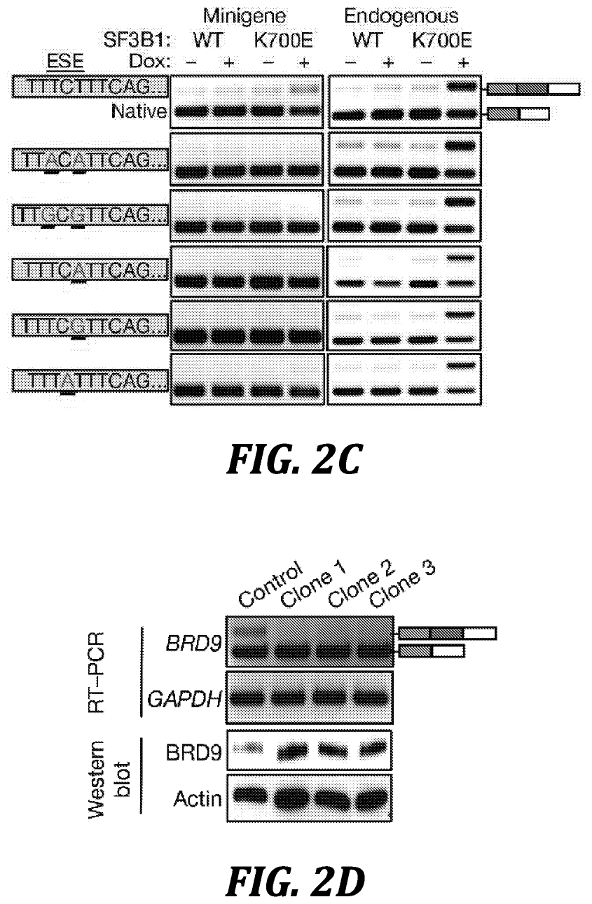

SF3B1 mutations promote the use of cryptic 3′ splice sites, probably by altering the normal role of SF3B1 in branchpoint recognition. The BRD9 branchpoints used in K562, MEL270 and T47D (breast cancer) cells that express mutant SF3B1 were therefore mapped (FIG. 2A) by generating a western blot for Flag, SF3B1 and endogenous BRD9 protein in MEL270 cells with doxycycline-inducible Flag-tagged wild-type SF3B1 or Flag-tagged SF3B1(K700E). Additionally, Sanger sequencing was conducted of RT lariats arising from inclusion or exclusion of the poison exon transcripts from the cells (not shown). The inclusion of the poison exon was associated with an unusually close branchpoint (close branchpoints are rare and normally inefficiently recognized). Mutating the aberrant branchpoint abolished poison exon recognition (FIG. 2B). Consistent with the lack of an obvious polypyrimidine tract upstream of the poison exon, neither U2AF1 nor U2AF2 knockdown compromised poison exon recognition, whereas introducing a poly(Y) tract resulted in robust inclusion of the poison exon even in wild-type cells (FIG. 2B) The results were also observed in K562 cells, where U2AF2, U2AF1 and histone H3 expression was monitored in cells transfected with siRNAs against U2AF1 and/or U2AF2 and the mean BRD9 poison exon inclusion was measured by quantitative PCR (qPCR) (not shown). Finally, a putative exonic splicing enhancer was identified that was essential for inclusion of the poison exon (FIG. 2C). The aberrant branchpoint, lack of a polypyrimidine tract, and exonic splicing enhancer were confirmed to be essential for poison exon recognition in the context of SF3B1^{R625H}, the most common mutation of SF3B1 in UVM. Briefly, these results were based on western blot analysis for Flag, SF3B1, BRD9 and actin in MEL270 cells expressing an empty vector or N-terminally Flag-tagged wild-type SF3B1, SF3B1^{R625H} or SF3B1^{K700E} cDNA. Additionally, RT-PCR was performed for analysis of BRD9 splicing in MEL270 cells expressing doxycycline-inducible empty vector, wild-type SF3B1, SF3B1(R625H) or SF3B1(K700E, including for the various minigene mutations (indicated in FIG. 2C) at the 5′ end of the poison exon. Disrupting the 3′ splice site and/or exonic splicing enhancer of the poison exon with CRISPR-directed mutagenesis markedly increased the levels of BRD9 protein in UVM cells with mutated SF3B1 (FIG. 2D), but had no effect on BRD9 splicing or expression in cells (i.e., MEL270 cells) with wild-type SF3B1.

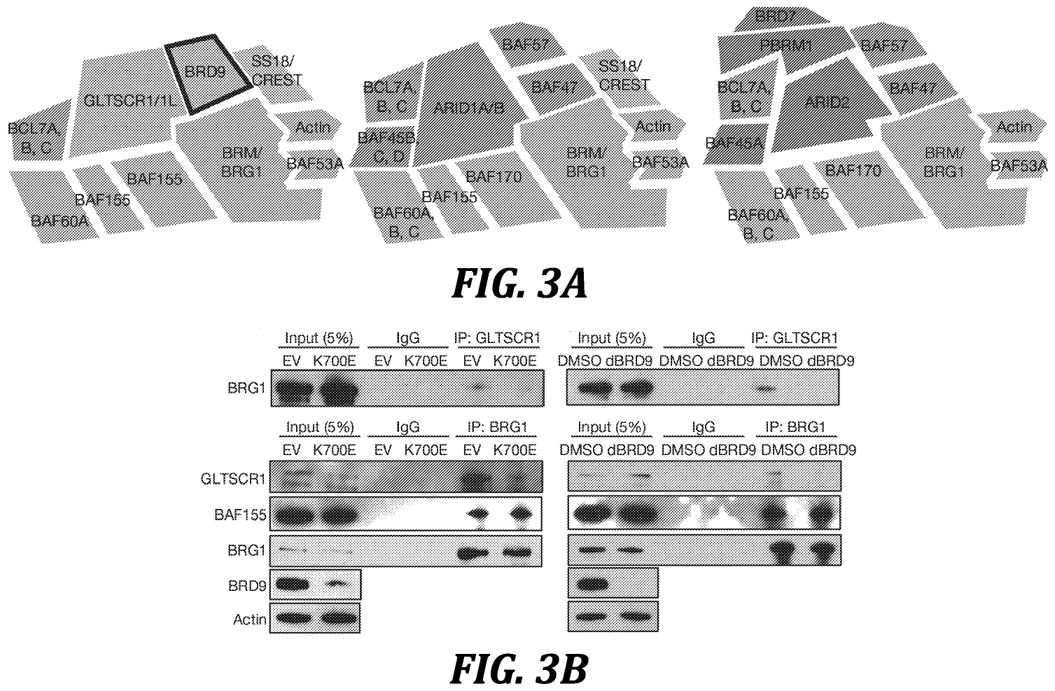

Several studies have recently described BRD9 as part of a non-canonical (nc) BAF complex, which is biochemically distinct from canonical BAF and polybromo-associated BAF (FIG. 3A) (see, e.g., Alpsoy, A. & Dykhuizen, E. C. Glioma tumor suppressor candidate region gene 1 (GLTSCR1) and its paralog GLTSCR1-like form SWI/SNF chromatin remodeling subcomplexes. J Biol. Chem. 293, 3892-3903 (2018); Michel, B. C. et al. A non-canonical SWI/SNF complex is a synthetic lethal target in cancers driven by BAF complex perturbation. Nat. Cell Biol. 20, 1410-1420 (2018), and Gatchalian, J. et al. A non-canonical BRD9-containing BAF chromatin remodeling complex regulates naive pluripotency in mouse embryonic stem cells. Nat. Commun. 9, 5139 (2018)). Although ncBAF is not recurrently mutated in cancer—unlike canonical BAF and polybromoassociated BAF—these data suggested that ncBAF is nonetheless frequently disrupted via SF3B1 mutations.

The consequences of BRD9 loss by SF3B1 mutations for ncBAF function were investigated. Immunoprecipitation and mass spectrometry to identify the chromatin-associated interaction partners of BRD9 in K562 cells specifically recovered ncBAF components. Briefly, western blot confirmed Flag-tagged BRD9 protein expression in 3× Flag-BRD9-expressing K562 cells. Rapid immunoprecipitation mass spectrometry of endogenous proteins (RIME) was used for purification and identification of the chromatin-associated interactions partners of BRD9. These results were confirmed by immunoblotting against shared and complex-specific components of canonical BAF, polybromo-associated BAF and ncBAF in K562 and UVM cells. Briefly, the immunoblotting was performed by cross-linking the cells and immunoprecipitating with IgG or Flag, followed by immunoblotting in K562 and MEL270 cells that express 3× Flag-BRD9. Expression of mutant, but not wild-type, SF3B1 reduced the levels of BRD9 protein and abolished interactions between BRG1 and GLTSCR1 while leaving interactions between BRG1 and BAF155 intact, which indicates that SF3B1 mutations specifically perturb ncBAF rather than disrupting all BAF complexes (FIG. 3B). Chemical degradation of BRD9 (Remillard, D. et al. Degradation of the BAF complex factor BRD9 by heterobifunctional ligands. Angew. Chem. Int. Edn Engl. 56, 5738-5743 (2017)) or BRD9 knockout similarly reduced the BRG1-GLTSCR1 interaction (FIG. 3B). This conclusion was supported by immunoprecipitation of GLTSCR1 (top) or BRG1 (bottom) followed by blotting with the indicated antibodies in K562 cells with CRISPR-mediated knockout (KO) of BRD9. The BRD9 domains that are necessary for ncBAF formation were identified by generating 3× Flag-BRD9 deletion mutants and testing for interactions with GLTSCR1 and GLTSCR1L. These experiments revealed that the DUF3512 domain of BRD9 mediates its interactions with GLTSCR1 and GLTSCR1L (not shown).

Next, it was determined how SF3B1 mutations altered ncBAF localization to chromatin. The genome-wide binding of the pan-BAF component BRG1 was mapped, and the ncBAF-specific components BRD9 and GLTSCR1, in MEL270 cells that express wild-type or mutant SF3B1. The same chromatin immunoprecipitation with sequencing (ChIP-seq) experiments was performed after treatment with dimethylsulfoxide (DMSO) or a BRD9 degrader to identify BRD9-dependent effects. BRD9 and GLTSCR1 exhibited substantial co-localization, consistent with their mutual requirement for ncBAF formation, and were found at a subset of the loci bound by BRG1 (not shown). BRD9 and GLTSCR1 bound to promoters, gene bodies, and probable enhancers, with focal binding at promoters relative to BRG1 (not shown). CTCF motifs exhibited notable co-localization with GLTSCR1, but only modest co-localization with BRG1.

The inventors next investigated how the depletion of BRD9, induced by SF3B1^{K700E} or by chemical degradation of BRD9, altered ncBAF localization. The genomic loci bound by GLTSCR1 in all samples were defined as constitutive sites. Conversely, genomic loci bound by GLTSCR1 in both controls (i.e., wild-type SF3B1 and DMSO) but not BRD9-depleted (mutant SF3B1 or BRD9 degradation) samples were defined as BRD9-sensitive sites. GLTSCR1 peaks were more sensitive to BRD9 loss than were BRG1 peaks, and CTCF motifs were uniquely enriched in BRD9-sensitive loci (P<1×10⁻⁸) versus constitutive GLTSCR1-bound loci. CTCF was similarly highly enriched at BRG1-bound loci that were BRD9-sensitive (P<1×10⁻⁵⁵). The BRD9 loss induced by SF3B1 mutations was concluded to cause specific loss of ncBAF at CTCF-associated loci.

Genes with BRD9-sensitive ncBAF binding in their promoters or enhancers were identified using Genomic Regions Enrichment of Annotations Tool (GREAT) analysis of genes near BRD9-sensitive and constitutive GLTSCR1-bound loci. It was found that BRD9 loss in UVM preferentially affects genes involved in apoptosis and cell growth, adhesion and migration. To understand how BRD9 loss altered gene expression, genes with promoters that exhibited BRD9-sensitive ncBAF binding and that were differentially expressed in patients with UVM with mutant versus wild-type SF3B1 were identified. Loss of ncBAF binding was associated with promotion as well as repression of gene expression, suggesting that ncBAF—similar to other SWI/SNF complexes—has both activating and repressive roles. Briefly, GLTSCR1 ChIP-seq (MEL270 cells) and RNA-seq (TCGA UVM cohort) around NFATC2IP were conducted. There was reduced GLTSCR1 binding in the promoter upon treatment with BRD9 degrader or expression of SF3B1^{K700E} NFATC2IP was significantly differentially expressed in UVM samples with SF3B1 mutations relative to wild-type samples. Similar results were obtained for SETD1A, where SETD1A was significantly differentially expressed in UVM samples with SF3B1 mutations relative to wild-type samples.

Several recent studies have reported that BRD9 is required for the survival of some cancer types, particularly cancers with mutations that affect polybromo-associated BAF and canonical BAF (Michel, B. C. et al. Nat. Cell Biol. 20, 1410-1420 (2018), Hohmann, A. F. et al. Sensitivity and engineered resistance of myeloid leukemia cells to BRD9 inhibition. Nat. Chem. Biol. 12, 672-679 (2016), and Brien, G. L. et al. Targeted degradation of BRD9 reverses oncogenic gene expression in synovial sarcoma. eLife 7, e41305 (2018)). Because BRD9 loss conferred a proliferative advantage to types of cancer with recurrent SF3B1 mutations (FIG. 1D), whether normalizing the levels of BRD9 might suppress the growth of SF3B1-mutant cells was investigated.

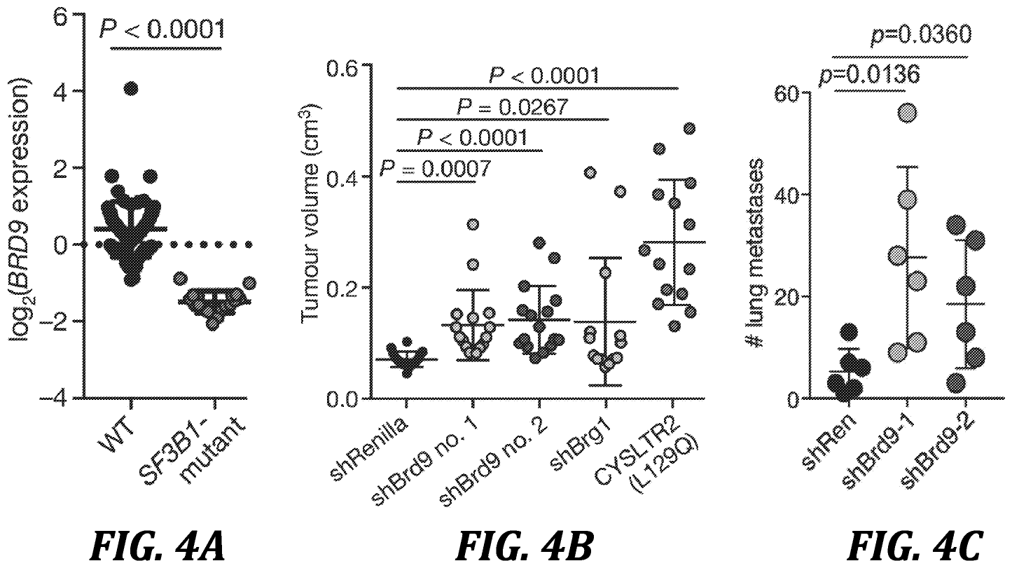

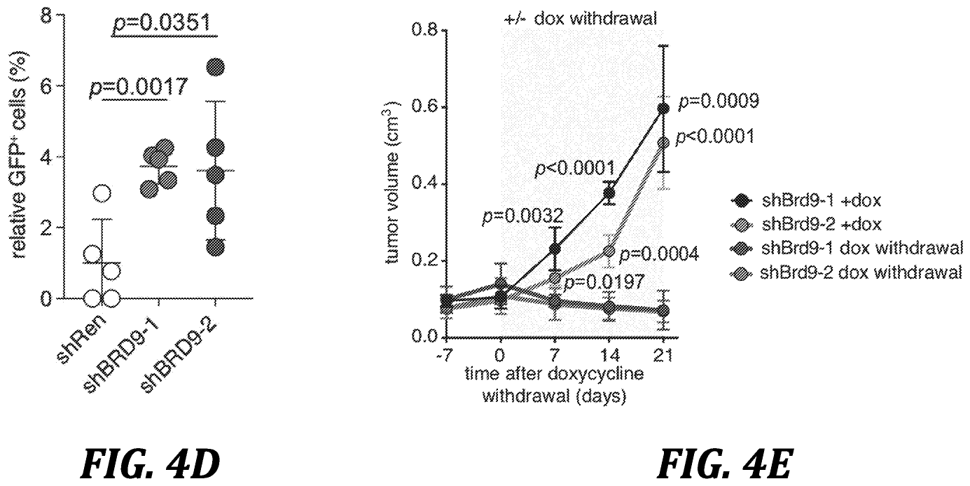

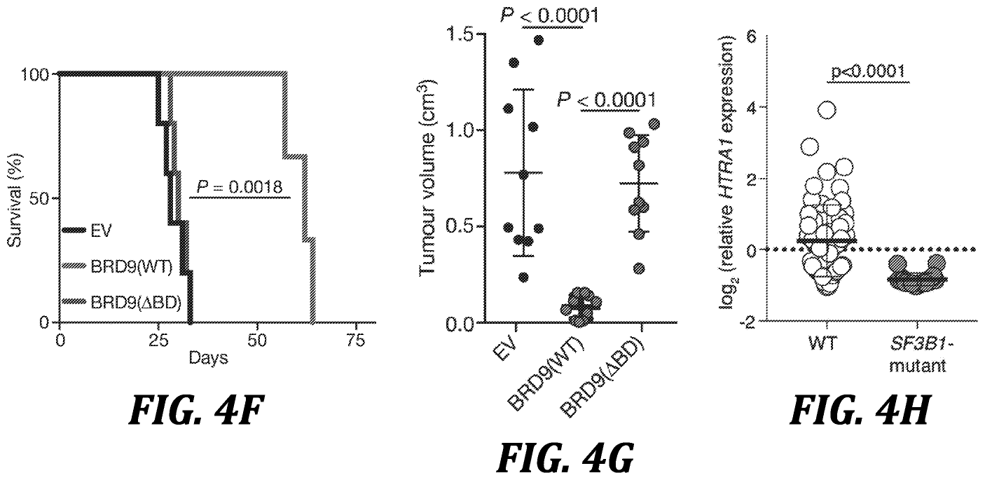

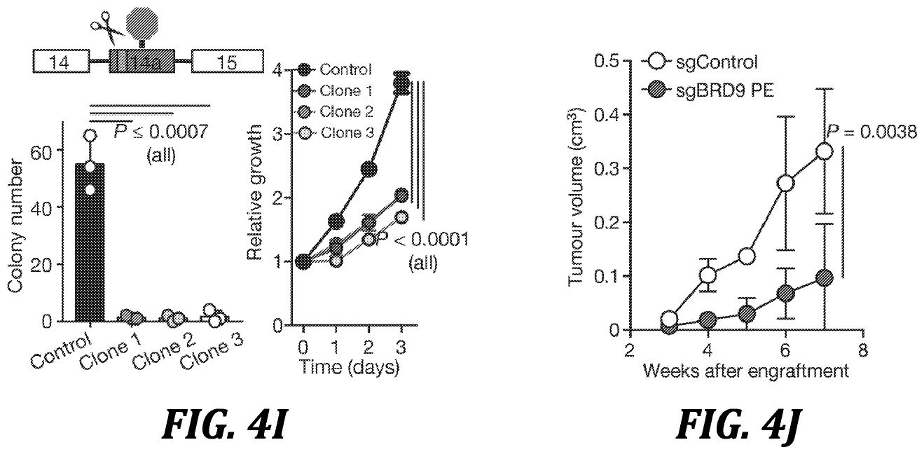

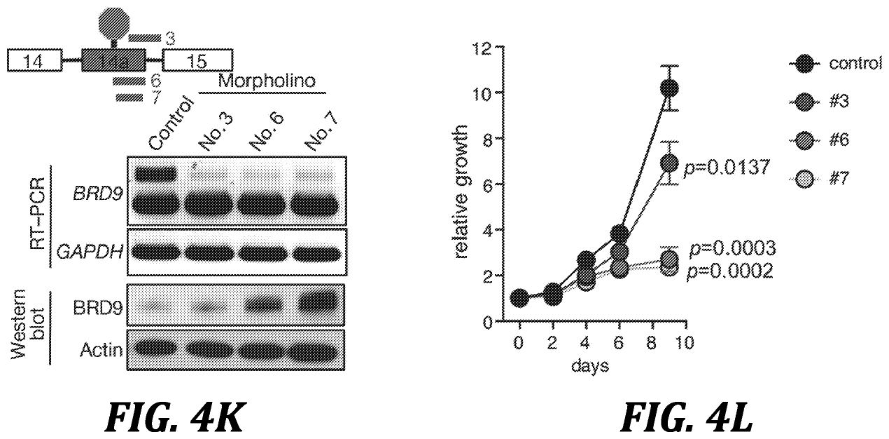

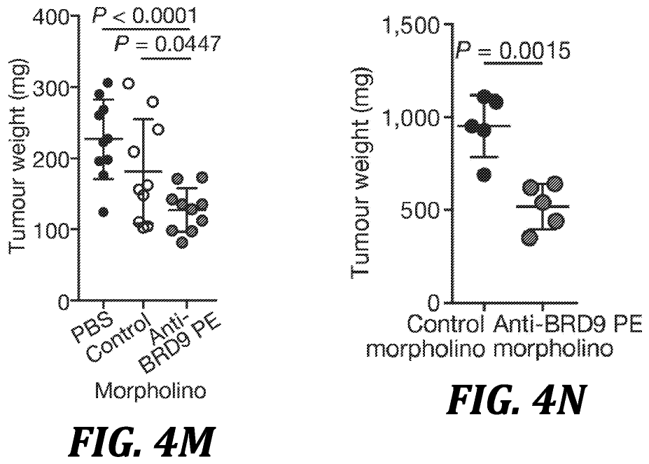

As SF3B1 is recurrently mutated in uveal (FIG. 4A), mucosal and cutaneous melanomas, it was first tested whether BRD9 loss induced melanomagenesis in vivo. Non-tumorigenic mouse melanocytes (Melan-a cells) were transduced, which require oncoprotein expression for sustained growth, with a non-targeting short hairpin RNA (shRNA), doxycycline-inducible shRNAs targeting Brd9 or Brg1 (also known as Smarca4) or a cDNA encoding the oncoprotein CYSLTR2(L129Q) (as a positive control; see Moore, A. R. et al. Recurrent activating mutations of G-protein-coupled receptor CYSLTR2 in uveal melanoma. Nat. Genet. 48, 675-680 (2016)). Knockdown of either Brd9 or Brg1 resulted in potent tumour growth (FIG. 4B), augmented melanocyte pigmentation (not shown), and expression of melanocyte-lineage-specific genes (Mitf, Dct, Pmel, and Tyrp1) in vivo.

Next, the inventors tested whether Brd9 expression influences metastasis. Brd9 knockdown significantly increased the number of pulmonary metastatic foci following intravenous injection of cells from a mouse model of melanoma (B16) or of human UVM (92.1) cells into mice (FIGS. 4C and 4D). By contrast, restoring Brd9 expression in established tumours in vivo, by withdrawing doxycycline, suppressed tumour growth (FIG. 4E). Similarly, ectopic expression of full-length BRD9, but not the bromodomain- or DUF3512-deletion mutants, suppressed the growth of UVM cell lines and xenografts (FIGS. 4F and 4G). This observation was also based on competition assays to measure the effects of expression of BRD9 WT and BRD9 ABD on growth of various melanoma cells over 21 days (not shown). Additional competition assays were performed in melanoma cells expressing EV, BRD9 FL, BRD9 ABD, and BRD9 ADUF over 10 days (not shown). These data demonstrate that loss of Brd9 promotes cell transformation, tumour maintenance, and metastatic progression, and that the bromodomain and DUF3512 domain of BRD9 are essential for its anti-proliferative effects.

The inventors next sought to understand how BRD9 loss promotes melanoma tumorigenesis. BRD9-bound genes that exhibited dysregulated expression were identified in samples from patients with UVM with mutated versus wild-type SF3B1, and in isogenic UVM cells with or without mutant SF3B1 and with or without forced loss of BRD9. HTRA1, a known tumour suppressor in melanoma, was the most downregulated gene in UVM (FIG. 4H). Western blots for Flag, SF3B1, HTRA1, BRD9 and actin in MEL270 cells (wild-type SF3B1), treated with DMSO, BRD9 degrader, Flag-SF3B1(WT) or Flag-SF3B1(K700E), and for HTRA1, BRD9 and actin in MEL202 cells (SF3B1^{R625G}) following CRISPR-Cas9-mediated mutagenesis of the BRD9 poison exon were generated (not shown). HTRA1 was suppressed by mutant SF3B1 expression and BRD9-degradation treatment of UVM cells with wild-type SF3B1, and mutagenesis of the BRD9 poison exon increased levels of HTRA1 in UVM cells with mutated SF3B1. HTRA1 is bound by ncBAF in UVM, and this binding is reduced by mutant SF3B1 as demonstrated in ChIP-seq analysis at the HTRA1 locus in MEL270 cells (not shown). HTRA1 knockdown promoted the growth of UVM cells with wildtype SF3B1, and ectopic expression of HTRA1 suppressed the growth of UVM cells with mutated SF3B1. Briefly, these conclusions were based, in part, on western blots for HTRA1 in MEL270 cells treated with shRNAs against HTRA1 or non-targeting control (not shown). Additionally, competition assays measuring the effect of shRNAs targeting HTRA1 on the growth of Cas9-expressing UVM cell lines with wild-type SF3B1 (not shown). Additionally, western blot analysis detected HTRA1 expression in MEL202 cells (SF3B1^{R625G}) following stable overexpression of an empty vector or HTRA1 (both with an MSCV-IRES-GFP vector) (not shown). Finally, colony numbers were assessed for MEL202 cells expressing empty vector or HTRA1 cDNA after 10 days of growth in agar (not shown). These data suggest that perturbation of ncBAF-dependent regulation of HTRA1 contributes to the pro-tumorigenic effects of BRD9 loss.

Next, the inventors tested whether correcting BRD9 mis-splicing suppressed tumorigenesis. CRISPR-based mutagenesis of the poison exon markedly slowed the growth of cells with mutated SF3B1, but not of wild-type cells, both in vitro and in vivo (FIGS. 2D, 4I, and 4J). Antisense oligonucleotides (ASOs) were then designed to block the inclusion of the BRD9 poison exon (FIG. 4K). SF3B1-mutated cells were treated with a non-targeting (control) or poison-exon-targeting ASO, and BRD9 splicing, BRD9 protein levels, and cell growth were measured. Each targeting ASO prevented the inclusion of the poison exon, increased the level of BRD9 protein, and suppressed cell growth relative to the control ASO (FIGS. 4K and 4L). The relative abilities of each ASO to restore BRD9 protein levels and suppress cell growth were strongly correlated, consistent with on-target effects.

The inventors therefore tested whether ASO treatment slowed tumour growth in vivo. SF3B1-mutated xenografts (derived from MEL202 cells) were treated with each ASO via intratumoral injection for 16 days. Treatment with the poison-exon-targeting ASO—but not with the non-targeting ASO—corrected BRD9 mis-splicing, significantly reduced tumour growth, and induced tumour necrosis (FIG. 4M; reduction of BRD9 with poison exon confirmed with RT-PCR (not shown)). A similar ASO efficacy was observed in a patient-derived xenograft model of rectal melanoma with the SF3B1R^{625C} mutation (FIG. 4N shows reduction in tumour weight; similar results were observed for tumour volume (not shown)). By contrast, when an identical experiment was performed with a patient-derived xenograft model of UVM that lacked an SF3B1 mutation, treatment with the poison-exon-targeting ASO had no effect (not shown). It was concluded that correcting BRD9 mis-splicing restores the tumour suppressor activity of BRD9 in cancers with SF3B1 mutations.

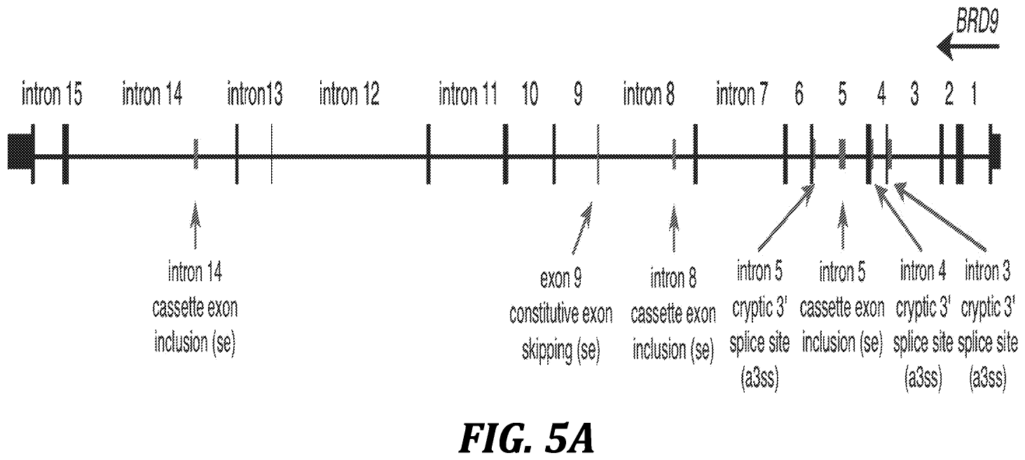

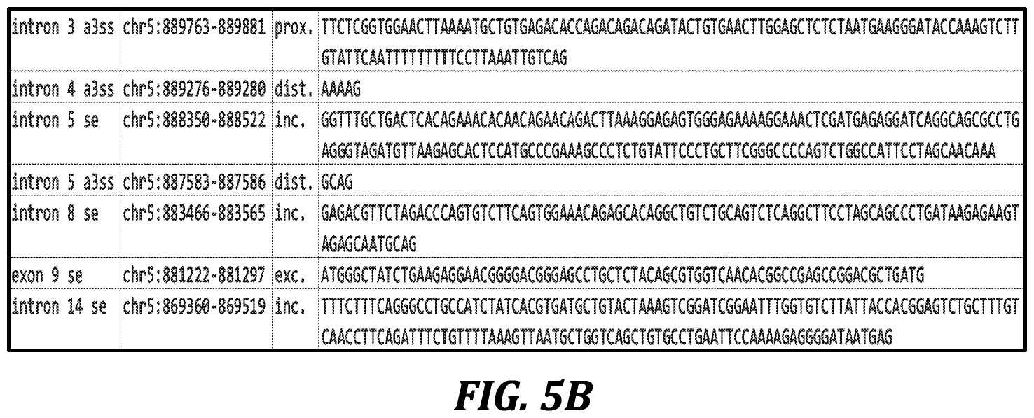

Although recognition of the BRD9 poison exon requires mutant SF3B1, BRD9 mis-splicing and ncBAF disruption may also have roles in cancers with wild-type SF3B1. Significant pan-cancer expression correlations were identified between BRD9 and many genes that encode RNA-binding proteins, as well as six additional BRD9 isoforms that are predicted to trigger NMD that are expressed in cancers with wild-type SF3B1 and are predictive of BRD9 expression (FIGS. 5A and 5B). This conclusion is also based on computational analysis of the levels of each NMD-inducing isoform relative to total BRD9 mRNA levels for each sample in various TCGA cohorts. Linear models were used to predict BRD9 gene expression on the basis of the relative levels of each NMD-inducing isoform. The coefficients were typically negative (as expected for NMD-inducing isoforms), with the exception of constitutive exon 9 skipping, for which the coefficients were generally positive—as expected for an event in which NMD is induced when a constitutive exon is excluded. The SF3B1-mutation-responsive poison exon in intron 14 dominates the fit for UVM, as expected. n=33 TCGA cohorts analysed. Furthermore, actual and predicted BRD9 expression levels for three TCGA cohorts were plotted and compared. RNA-Seq read coverage plots were generated from patient samples from the TCGA cohorts. A promoter polymorphism associated with decreased GLTSCR1 (also known as BICRA) expression has been identified as a common risk allele for acute myeloid leukaemia.

Because BRD9 mis-splicing was observed in a range of cancer types that carry distinct SF3B1 mutations, targeting BRD9 mis-splicing could be a productive pan-cancer therapy. Although ASO treatment merely restored BRD9 mRNA and BRD9 protein to normal levels, a strong suppression of tumour growth was nonetheless observed. The functional effect of correcting BRD9 mis-splicing was particularly notable given that the UVM models used here contain hundreds of other mis-splicing events and multiple pro-tumorigenic mutations. Given recent clinical successes with treating spinal muscular atrophy and other diseases with ASOs, the tumour-suppressive effects of correcting BRD9 mis-splicing suggest that oligonucleotide-based therapy may prove similarly promising for treating cancers with spliceosomal mutations.

### Methods and Materials

Sample sizes for xenograft experiments were chosen on the basis of published studies of known oncogenic drivers of relevant models (for example, expression of the oncoprotein CYSLTR2(L129Q) in Melan-a cells). Mice were randomly assigned to experimental groups. The data presented did not require the use of blinding.

Cell Lines and Tissue Culture

All cell lines underwent short-tandem repeat testing (ATCC) and Memorial Sloan Kettering integrated mutation profiling of actionable cancer targets (MSK IMPACT) genetic analysis₂₉ to evaluate for spliceosome-gene mutational status and status of recurrently mutated genes in cancer. HEK293T cells were grown in DMEM with 10% FCS. Ba/F3 cells and Melan-a cells were grown in RPMI with 10% FCS with 1 ng/ml IL-3 (PeproTech, 213-13) and 200 nM TPA (Sigma-Aldrich), respectively, unless noted otherwise. The K562 and NALM-6 isogenic cell lines (engineered to express SF3B1^{K700E}, SF3B1^{K666N} or SF3B1^{K700K} (wild-type control for genome engineering) from the endogenous SF3B1 locus) were cultured in RPMI with 10% FCS and their generation has previously been described (Seiler, M. et al. H3B-8800, an orally available small-molecule splicing modulator, induces lethality in spliceosome-mutant cancers. Nat. Med. 24, 497-504 (2018)). MEL270, MEL285 and RN2 cell lines were cultured in RPMI with 10% FCS. MEL202, 92-1 and SK-MEL30 cells were grown in RPMI with 10% FCS and 1% GlutaMAX (Gibco). UPMD1 and UPMD2 cells were grown in Ham F-12 with 10% FCS. CFPAC1 cells were cultured in IMDM with 10% FCS. KPC, Miapaca2 and B16 cells were cultured in DMEM with 10% FCS. Panc 05.04 cells were grown in RPMI with 20% FCS and 20 units per millilitre human recombinant insulin. T47D cells were cultured in RPMI1640 supplemented with 10% fetal bovine serum (Corning), 100 μg/ml penicillin, 100 mg/ml streptomycin (Corning), and 4 mM glutamine. All of the cell culture media included penicillin (100 U/ml) and streptomycin (100 μg/ml).

Primary human samples and human patient-derived xenograft models Studies were approved by the Institutional Review Boards (IRBs) of Memorial Sloan Kettering Cancer Center (MSK), informed consent was obtained from all subjects (under MSK IRB protocol 06-107) and studies were conducted in accordance to the Declaration of Helsinki protocol. Patients provided samples after their informed consent, and samples of primary human de-identified chronic lymphocytic leukaemia derived from whole peripheral blood or bone marrow mononuclear cells were used. Patient-derived xenograft models were performed using tumour biopsies from de-identified patients under MSK IRB protocol 14-191. Genomic alterations in melanoma tumour biopsies and chronic lymphocytic leukaemia cells were analysed using the MSK IMPACT (Cheng, D. T. et al. Memorial Sloan Kettering-integrated mutation profiling of actionable cancer targets (MSK-IMPACT): a hybridization capture-based next-generation sequencing clinical assay for solid tumor molecular oncology. J Mol. Diagn. 17, 251-264 (2015)) assay or FoundationOne Heme (Leeksma, A. C. et al. Clonal diversity predicts adverse outcome in chronic lymphocytic leukemia. Leukemia 33, 390-402 (2019)) assay, both as previously described. Patient samples were anonymized by the Hematologic Oncology Tissue Bank of MSK (for chronic lymphocytic leukaemia samples) and the MSK Antitumour Assessment Core Facility (for patient-derived xenograft samples).

Mice

All mice were housed at Memorial Sloan Kettering Cancer Center (MSKCC). All mouse procedures were completed in accordance with the Guidelines for the Care and Use of Laboratory Animals and were approved by the Institutional Animal Care and Use Committees at MSKCC. All mouse experiments were performed in accordance with a protocol approved by the MSKCC Institutional Animal Care and Use Committee (11-12-029). SCID mice (Jackson Laboratories stock no. 001303) were used for all human cell line xenografts and NSG mice (Jackson Laboratories stock no. 005557) were used for patient-derived xenografts. For all mouse experiments, the mice were monitored closely for signs of disease or morbidity daily and were killed when they showed a volume of the visible tumour formation above 1 cm³, failed to thrive, experienced weight loss >10% total body weight or showed open skin lesions, bleeding or any signs of infection. These limits were not exceeded in any experiments.

HA-Tag Knock-In into Endogenous BRD9

The following guide RNA sequence targeting the BRD9 transcriptional start site was selected using the optimized CRISPR design tool (//crispr.mit.edu): CGAGTGGCGCTCGTCCTACG (SEQ ID NO: 13). DNA oligonucleotides were purchased from IDT and cloned into the px458-GFP vector. For homologous recombination, a custom IDT Ultramer 197-bp repair template (single-stranded donor DNA) with the HA sequence (TACCCATACGATGTTCCAGATTACGCT (SEQ ID NO:14)) directly following the BRD9 start codon was purchased. This 197-bp fragment contained two silent mutations, one to remove the PAM site (AGG>AAG) and another to introduce an XhoI restriction enzyme (CTCGGG>CTCGAG) site upstream of the HA tag. The 197-bp fragment also contained 83 bp of homology to the BRD9 5′ UTR upstream of the HA tag and 87 bp of homology to the BRD9 exon 1 downstream of the start codon. Five micrograms of the targeting construct and 500 nM of the repair template were nucleofected into K562 SF3B1^{K700E} cells and MEL270 cells using the Lonza Nucleofector V kit and Program T-003 on the nucleofector device. Nucleofected cells were single cell-sorted on the basis of GFP positivity 48 h after nucleofection. Clones were screened for the presence of successful HA insertion by BRD9 exon 1 PCR and subsequent restriction enzyme digestion with XhoI and direct Sanger sequencing. A single positive clone containing the HA coding sequencing was selected to carry out further studies. The sgRNAs were CACCCGAGTGGCGCTCGTCCTACG (top) (SEQ ID NO:15) and AAACCGTAGGACGAGCGCCACTCG (bottom) (SEQ ID NO: 16); the single-strand donor DNA was CCAGGGGGCGGTGGCGGCCAAGGTCCGACCGGGTGCCAGCTGTTCCCAGCCC CCGCCTCGAGCCCGCCGCCGGCGCCGCCATGTACCCATACGATGTTCCAGATT ACGCTGGCAAGAAGCACAAGAAGCACAAGGCCGAGTGGCGCTCGTCCTACG AAGGTGAGGCGGCGGCGCTTTGTGACGCGCGGCGGGCGGGG (SEQ ID NO:17); the PCR primers were forward (fwd) AGCGAGCTCGGCAACCTCG (SEQ ID NO:18) and reverse (rev) CTTCAGGACTAGCTTTAGAGGC (SEQ ID NO:19); the Sanger sequence primer was rev TGCAGCCTCGAACCCAGAAC (SEQ ID NO:20).

Overexpression of SF3B1 cDNA in K562 Cells

Two micrograms of PiggyBac Transposase construct (CMV-PB-Transposase-IRES-TK-HSV) and 6 μg of wildtype SF3B1 (ITR-CAG-Flag-SF3B1WT-IRES-Puro-ITR) or SF3B1^{K700E} (ITR-CAGFlag-SF3B1^{K700E}-IRES-Puro-ITR) cDNA constructs were electroporated into 2×10⁶ cells (in 200 μl volume) using the Amaxa Nucleofector Protocol (Program T-003) according to manufacturer instructions (Lonza). Puromycin selection (1 μg/ml) was initiated 4 days after electroporation to select for cells that successfully incorporated the constructs. Sanger sequencing was performed to confirm successful integration of the cDNA plasmid using the following primers: fwd, TCCAATCAAAGATCTTCTTCCAA (SEQ ID NO:21) and rev, GAGCAGGTTTCTGCAACGAT (SEQ ID NO:22).

RT-PCR and Quantitative RT-PCR

Total RNA was isolated using RNeasy Mini or Micro kit (Qiagen). For cDNA synthesis, total RNA was reverse-transcribed to cDNA with SuperScript VILO cDNA synthesis kit (Life Technologies). The resulting cDNA was diluted 10-20 fold before use. Quantitative RT-PCR (qRT-PCR) was performed in 10-μl reactions with SYBR Green PCR Master Mix. All qRT-PCR analysis was performed on an Applied Biosystems QuantStudio 6 Flex Cycler (ThermoFisher Scientific). Relative gene expression levels were calculated using the comparative CT method. Primers used in RT-PCR reactions were: BRD9 (human) fwd, GCAATGACATACAATAGGCCAGA (SEQ ID NO:23) and rev, GAGCTGCCTGTTTGCTCATCA (SEQ ID NO:24); Brd9 (mouse) fwd, TTGGAGATGGAAGTCTGCTCT (SEQ ID NO:25) and rev, GCAACTTGCTAGACAGTGAACT (SEQ ID NO:26); BRD9 poison exon (human) fwd, AGCTCTGTTCTGGAGTTCATG (SEQ ID NO:27) and rev, CTGAAGAAACTCATAGGGGTCGTG (SEQ ID NO:28); Brd9 poison exon (mouse) fwd, GGCCCTGTTCTGGACTTCATG (SEQ ID NO:29) and rev, CTGAAGGAATTCATAAGGGTCGTG (SEQ ID NO:30); BRD9 poison exon inclusion for small interfering RNA (siRNA) experiment (human) fwd, CAGCAGCTCTGTTCTGGAGT (SEQ ID NO:31) and rev: CCTGAAAGAAACCAGAGAGCTG (SEQ ID NO:32); BRD9 poison exon exclusion for siRNA experiment (human) fwd, CAGCAGCTCTGTTCTGGAGT (SEQ ID NO:33) and rev, TCACCTTCCCCAGAGAGCTG (SEQ ID NO:34); EPB49 (also known as DMTN) cassette exon inclusion (human) fwd, GCCTGCAGAACGGAGAGG (SEQ ID NO:35) and rev: ACCACTAGCATTTCATAGGGATAGATCT (SEQ ID NO:36); EPB49 cassette exon exclusion (human) fwd, GCCTGCAGATCTATCCCTATGAAAT (SEQ ID NO:37) and rev, CTCAAGCCGCATCCGATCC (SEQ ID NO:38); BRD9 poison exon for mRNA half-life experiment (human) fwd, GTTGGGGACACCCTAGGAGA (SEQ ID NO:39), rev (exclusion-specific), CTTCACCTTCCCCAGAGAGC (SEQ ID NO:40) and rev (inclusion-specific), CCCTGAAAGAAACCAGAGAGC (SEQ ID NO:41); 18S rRNA (human) fwd CTACCACATCCAAGGAAGCA (SEQ ID NO:42) and rev, TTTTTCGTCACTACCTCCCCG (SEQ ID NO:43); Mitf (mouse) fwd, CCAACAGCCCTATGGCTATGC (SEQ ID NO:44) and rev: CTGGGCACTCACTCTCTGC (SEQ ID NO:45); Dct (mouse) fwd, GTCCTCCACTCTTTTACAGACG (SEQ ID NO:46) and rev, ATTCGGTTGTGACCAATGGGT (SEQ ID NO:47); Pmel (mouse) fwd, GAGCTTCCTTCCCGTGCTT (SEQ ID NO:48) and rev, TGCCTGTTCCAGGTTTTAGTTAC (SEQ ID NO:49); Tyrp1 (mouse) fwd, CCCCTAGCCTATATCTCCCTTTT (SEQ ID NO:50) and rev, TACCATCGTGGGGATAATGGC (SEQ ID NO:51); GAPDH (human) fwd, GGAGCGAGATCCCTCCAAAAT (SEQ ID NO:52) and rev, GGCTGTTGTCATACTTCTCATGG (SEQ ID NO:53); and Gapdh (mouse) fwd, AGGTCGGTGTGAACGGATTTG (SEQ ID NO:54) and rev, TGTAGACCATGTAGTTGAGGTCA (SEQ ID NO:55).

mRNA Stability Assay

For mRNA half-life measurement using qRT-PCR, K562 and NALM-6 cells with isogenic SF3B1^{K700E} mutations were infected with anti-UPF1 shRNAs or control shRNA, and treated with 2.5 μg/ml actinomycin D (Life Technologies) and collected at 0, 2, 4, 6 and 8 h (using methods as described Kim, E. et al. SRSF2 mutations contribute to myelodysplasia by mutant-specific effects on exon recognition. Cancer Cell 27, 617-630 (2015)). BRD9 poison exon inclusion or exclusion and 18S rRNA mRNA levels were measured by qRT-PCR.

Western Blotting

For western blotting, the following antibodies to the following proteins were used: BRD9 (Bethyl Laboratories A303-781A and Active Motif 61538), SF3B1/Sap-155 (MBL D221-3), Flag-M2 (Sigma-Aldrich F-1084), β-actin (Sigma-Aldrich A-5441), GLTSCR1 (Santa Cruz Biotechnology sc-515086), GLTSCR1L (Thermo Fisher Scientific PA5-56126), BRM (Bethyl Laboratories A303-015A), BRG1 (Santa Cruz Biotechnology sc-17796), BAF155 (Santa Cruz Biotechnology sc-48350), BAF60A (Santa Cruz Biotechnology sc-135843), BAF47 (Santa Cruz Biotechnology sc-166165), ARID1A (Santa Cruz Biotechnology sc-373784), ARID2 (Santa Cruz Biotechnology sc-166117), BRD7 (Thermo Fisher Scientific PA5-49379), U2AF2 (Bethyl Laboratories A303-665A), U2AF1 (Bethyl Laboratories A302-080A), histone H3 (Abcam ab1791), HTRA1 (R&D Systems MAB2916-SP). All primary antibodies for western blotting were diluted to a final concentration of 1:500 to 1,000, in either 5% BSA (Sigma-Aldrich) in 0.05% TBS-Tween 20 (TBS-T) or 5% skim milk in 0.05% TBS-T. Nuclear extracts were quantified using BCA and 1 mg protein (1 mg ml⁻¹ in immunoprecipitation buffer supplemented with protease inhibitors) was used per immunoprecipitation. Proteins were incubated for 3 h with 2-5 μg of antibody or with protein A/G PLUS-Agarose (Santa Cruz Biotechnology sc-2003) with rotation at 4° C. After washing three times with Pierce IP Lysis Buffer (Thermo Fisher Scientific 87787), immunoprecipitated proteins were eluted with Pierce Lane Marker Reducing Sample Buffer (Thermo Fisher Scientific 39000) and loaded onto 4-12% Bis-Tris NuPAGE Gels (Life Technologies).

ChIP

For ChIP-seq studies in MEL270 cells, antibodies to endogenous BRG1 (Abcam EPNCIR111A, lot no. GR3208604-8), GLTSCR1 (Santa Cruz SC-240516, lot no. A2313) and BRD9 (Abcam ab137245) were used, and ChIP was performed as described in Michel, B. C. et al. Nat. Cell Biol. 20, 1410-1420 (2018). MEL270 cells transduced with empty vector, doxycycline-inducible wild-type SF3B1 cDNA, or doxycycline-inducible SF3B1^{K700E} cDNA in the backbone of pInducer20, were treated with doxycycline (1 μg/ml) plus BRD9 degrader (Remillard, D. et al. Degradation of the BAF complex factor BRD9 by heterobifunctional ligands. Angew. Chem. Int. Edn Engl. 56, 5738-5743 (2017)) (250 nM) or DMSO for 72 h before crosslinking.

Mass Spectrometry

For anti-Flag-BRD9 ChIP followed by mass spectrometry, K562 cells transduced with empty vector or 3× Flag-tagged BRD9 were grown in RPMI with 10% FCS. Ten million cells were crosslinked according to the manufacturer's instruction (Active Motif) and as described in Mohammed, H. et al. Endogenous purification reveals GREB1 as a key estrogen receptor regulatory factor. Cell Rep. 3, 342-349 (2013). Cells were fixed with 10% methanol-free formaldehyde (Sigma, F-8775) for 8 min and quenched with 0.125 M glycine (Sigma, G-7403). Chromatin was isolated by the addition of lysis buffer, followed by disruption with a Dounce homogenizer. Lysates were sonicated and the DNA sheared to an average length of 300-500 bp. Genomic DNA (input) was prepared by treating aliquots of chromatin with RNase, proteinase K and heat for reverse-crosslinking, followed by ethanol precipitation. Pellets were resuspended and the resulting DNA was quantified on a NanoDrop spectrophotometer. Extrapolation to the original chromatin volume allowed quantification of the total chromatin yield. An aliquot of chromatin (150 μg) was precleared with protein G agarose beads (Invitrogen). Proteins of interest were immunoprecipitated using 15 μg of antibody against Flag and protein G magnetic beads. Protein complexes were washed and trypsin was used to remove the immunoprecipitated from beads and digest the protein sample. Protein digests were separated from the beads and purified using a C18 spin column (Harvard Apparatus). The peptides were vacuum-dried using a SpeedVac. Digested peptides were analysed by liquid chromatography and tandem mass spectrometry on a Thermo Scientific Q Exactive Orbitrap mass spectrometer in conjunction with a Proxeon Easy-nLC II HPLC (Thermo Scientific) and Proxeon nanospray source.

Protein identifications were accepted if they contained at least one identified peptide. Proteins that contained similar peptides and could not be differentiated on the basis of tandem mass spectrometry analysis alone were grouped to satisfy the principles of parsimony. Proteins sharing significant peptide evidence were grouped into clusters. The final list was generated by taking all proteins with a spectral count of five and above from each replicate reaction and comparing them in a Venn diagram against IgG control replicates. Proteins unique to both experimental replicates were then applied to the PANTHER database for protein ontology results.

shRNA Experiments

Cells were transduced with a doxycycline-inducible LT3GEPIR lentiviral vector, T3G-GFP-mirE-PGK-Puro-IRES-rtTA3 (Fellmann, C. et al. An optimized microRNA backbone for effective single-copy RNAi. Cell Rep. 5, 1704-1713 (2013)), expressing shRNAs against BRD9 or a non-targeting shRNA against Renilla. shRNAs were induced with the addition of doxycycline (2.0 μg/ml, Sigma Aldrich). All shRNAs were designed using the SplashRNA algorithm (Pelossof, R. et al. Prediction of potent shRNAs with a sequential classification algorithm. Nat. Biotechnol. 35, 350-353 (2017)). The shRNA sequences are: BRD9 shRNA no. 1 (human, shBRD9_352): TTTATTATCATTGAATATCCAG (SEQ ID NO:56); BRD9 shRNA no. 2 (human, shBRD9_353): TTTTATTATCATTGAATATCCA (SEQ ID NO:57); Brd9 shRNA no. 1 (mouse, shBrd9_511): TTTATTATCATTGAATACCCAG (SEQ ID NO:58); Brd9 shRNA no. 2 (mouse, shBrd9_512): TTTTATTATCATTGAATACCCA (SEQ ID NO:59); HTRA1 shRNA no. 1 (human, shHTRA1_1192): TTTTTAATCTTATCAGATGGGA (SEQ ID NO:60); HTRA1 shRNA no. 2 (human, shHTRA1_1669): TGAACAAACAAAATGGCAGTCA (SEQ ID NO:61); and HTRA1 shRNA no. 3 (human, shHTRA1_1898): TTCTATCTACGCATTGTATCGA (SEQ ID NO:62).

siRNA Transfections.

K562 cells were transfected with a non-targeting control siRNA (Dharmacon, D-001810-01, target sequence: UGGUUUACAUGUCGACUAA (SEQ ID NO:63)), an siRNA pool against U2AF1 (Dharmacon ON-TARGETplus SMARTpool, L-012325-01) or an siRNA pool against U2AF2 (Dharmacon ON-TARGETplus SMARTpool, L-012380-02) using the Nucleofector II device from Lonza with the Cell Line Nucleofector Kit V (program T16). RNA and protein were extracted 48 h after transfection. cDNA was produced using 1 μg of RNA and the Superscript III first strand synthesis system (Thermo Fisher, 18080051).

In Vitro Competition Assay.

For the competition assay to evaluate the cellular effect of sgRNA against BRD9 or Brd9, cell lines were transduced with LentiCas9-Blast (Addgene no. 52962) and then single-cell-sorted into 96-well plates. Among these clones, single clones with strong Cas9 expression were used, which was confirmed by western blotting. Cas9-expressing cells were lentivirally transduced with iLenti-guide-GFP vectors subcloned with the gRNA sequences, in which sgRNA expression was linked to GFP expression. The percentage of GFP-expressing cells was then measured over time after infection using BD LSRFortessa. The GFP-positive rates in living cells at each point compared to that of day 2 were calculated. Similarly, to evaluate the cellular effect of BRD9 fragment cDNA, HTRA1 cDNA or shRNAs against BRD9 or HTRA1, GFP-positive rates were measured after transducing pMIGII-backbone plasmids (cDNA) and LT3GEPIR plasmids (shRNA), respectively, into melanoma cell lines. The sgRNAs were as follows: BRD9 sgRNA no. 1 (human): ACTCCAGTTACTATGATGAC (SEQ ID NO:64), BRD9 sgRNA no. 2 (human): AGAGAGGGAGCACTGTGACA (SEQ ID NO:65), BRD9 sgRNA no. 3 (human): AGATACCGTGTACTACAAGT (SEQ ID NO:66), Brd9 sgRNA no. 1 (mouse): ATTAACCGGTTTCTCCCGGG (SEQ ID NO:67), Brd9 sgRNA no. 2 (mouse): GGAACACTGCGACTCAGAGG (SEQ ID NO:68), Brd9 sgRNA no. 3 (mouse): ACTTGCTAGACAGTGAACTC (SEQ ID NO:69), and control (scrambled sgRNA): ACGGAGGCTAAGCGTCGCAA (SEQ ID NO:70).

CRISPR Enrichment Screening for NMD Targets.

First, Ba/F3 cells were transduced with LentiCas9-Blast (Addgene no. 52962) and single-cell-sorted into 96-well plates. Among these clones, a single clone with strong Cas9 expression was used. The sgRNA library of NMD targets in SF3B1-mutant cells were amplified and packaged as lentivirus. The library includes 4 sgRNAs against each target gene (a total of 274 genes), 100 control sgRNAs and positive-control sgRNAs against Pten. Ba/F3 cells were transduced with the lentivirus-carrying sgRNA library produced by 293FT cells, and puromycin selection (2 μg/ml) was performed in IL-3-containing medium for 7 days. Then, IL-3 was washed out (on day 0) and the surviving cells were collected 7 days after IL-3 depletion (day 7). Cell pellets were lysed and genomic DNA was extracted (Qiagen), and quantified by Qubit (Thermo Scientific). A quantity of gDNA covering a 1,000× representation of gRNA was PCR-amplified using Q5 high-fidelity polymerase (NEB cat. no. M0491) to add Illumina adapters and multiplexing barcodes. Amplicons were quantified by Qubit and Bioanalyzer (Agilent) and sequenced on Illumina HiSeq 2500. Sequencing reads were aligned to the screened library; counts were computed for each gRNA and counts for each sgRNA were compared between days 0 and 7 after cytokine depletion. For the probe-level analysis, a negative binomial generalized log-linear model was fitted and a likelihood ratio test was performed with glmFit and glmLRT in the Bioconductor edgeR package. For the gene-level analysis, the CAMERA test as implemented in edgeR (Robinson, M. D., et al. edgeR: a Bioconductor package for differential expression analysis of digital gene expression data. Bioinformatics 26, 139-140 (2010); McCarthy, D. J., et al. Differential expression analysis of multifactor RNA-seq experiments with respect to biological variation. Nucleic Acids Res. 40, 4288-4297 (2012); Wu, D. & Smyth, G. K. Camera: a competitive gene set test accounting for inter-gene correlation. Nucleic Acids Res. 40, e133 (2012)) was used. FDR values were computed using the Benjamini-Hochberg method.

CRISPR-Directed Mutations

Cas9-expressing MEL202 and MEL270 cells were transduced with iLenti-guide-Puro vector targeting the 5′ end of the BRD9 poison exon. The sgRNA used to induce mutations was: AAAATACTCAGTTTCTTTCA (SEQ ID NO:71). Single-cell sorting was performed into a 96-well plate using a BD FACSAria III cell sorter after puromycin selection (2.0 μg/ml for 7 days). The mutations caused by Cas9 and the sgRNA were confirmed by PCR amplification, followed by Sanger sequencing. The PCR primers were fwd, TGTTGGGTCAGGAAGAGACTTG (SEQ ID NO:72) and rev, CCATGGACTGAACGGATTCC (SEQ ID NO:73); and the Sanger sequencing primer was fwd, TGTTGGGTCAGGAAGAGACTTG (SEQ ID NO:74).

Colony-Forming Assays.

Single-cell suspensions were prepared from MEL202 and MEL270 cells with or without CRISPR-mediated insertions and deletions (indels), 3,000 cells from each cell line were plated in triplicates in 6-well treated plates and colonies were enumerated 10 days later. After 10 days, colonies were fixed with 3.7% paraformaldehyde for 5 min and stained in a solution of 0.05% crystal violet for 30 min at room temperature and washed in PBS and tap water.

In Vitro Cell Viability Assays.

Cells were seeded in white flat-well 96-well plates (Costar) at a density of 1,000 cells per well. ATP luminescence readings were taken every 24 h after seeding, using Cell Titer Glo (Promega) according to the manufacturer's instructions.

BRD9 Minigene Assay.

Putative exonic splicing enhancers with SpliceAid 2 (Piva, F., Giulietti, M., Burini, A. B. & Principato, G. SpliceAid 2: a database of human splicing factors expression data and RNA target motifs. Hum. Mutat. 33, 81-85 (2012)) were identified. SF3B1 wild-type MEL270 and T47D cells transduced with Flag-SF3B1(WT) or Flag-SF3B1(K700E) (in the backbone of pInducer20 (Addgene no. 44012)) were used. After drug selection with neomycin (Thermo Fisher Scientific 10131027), the selected cells were treated with 1 μg/ml doxycycline (Sigma D9891). The BRD9 minigene construct was generated by inserting the DNA fragment containing the BRD9 genomic sequence from exon 14 to exon 15 in between the BamHI and AgeI restriction sites in the FRES plasmid (Addgene 62377) via Gibson assembly. BRD9 minigene mutagenesis was performed with the Agilent QuikChange II site-directed mutagenesis kit with specific primers according to the manufacturer's directions. For transient transfection experiments, cells were seeded into a 24-well plate one day before transfection of BRD9 minigene constructs in the presence of X-tremeGENE HP DNA transfection reagent (Roche) according to the manufacturer's directions. Forty-eight hours after transfection, cells were collected and RNA was extracted using Qiagen RNeasy mini kit. Minigene-derived and endogenous BRD9 transcripts were analysed by RT-PCR using specific primers. Primers and oligonucleotides used in RT-PCR reactions were: cloning fwd GGACCCAGTACCAGGATCCGTTGGGGACACCCTAGGAG (SEQ ID NO:75) and rev CTTGGAGGAGCGCACACCGGTCAGGTGGTGCTGGTCCCTC (SEQ ID NO:76); RT-PCR fwd (minigene) GGATTACAAGGATGACGATGAC (SEQ ID NO:77), fwd (endogenous) CATGAAGCCTCCAGATGAAG (SEQ ID NO:78) and rev (common) CTTCGTCGTCTCATCCAAGTTC (SEQ ID NO:79); mutagenesis (AAAA to TTTT) fwd CAAAGGGATATATTTTGAGATTTTTTACTCAGTTTCTTTCAGG (SEQ ID NO:80) and rev CCTGAAAGAAACTGAGTAAAAAATCTCAAAATATATCCCTTTG (SEQ ID NO:81); mutagenesis (AAAA to ATAT) fwd CAAAGGGATATATTTTGAGATATATTACTCAGTTTCTTTCAGG (SEQ ID NO:82) and rev CCTGAAAGAAACTGAGTAATATATCTCAAAATATATCCCTTTG (SEQ ID NO:83); mutagenesis (GATAAAA to TTTTTTT) fwd GTCAAAGGGATATATTTTGATTTTTTTTACTCAGTTTCTTTCAGG (SEQ ID NO:84) and rev; mutagenesis (TTTCT to TTACA at exon 14a) fwd GAGATAAAATACTCAGTTACATTCAGGGCCTGCCATCTATC (SEQ ID NO:85) and rev GATAGATGGCAGGCCCTGAATGTAACTGAGTATTTTATCTC (SEQ ID NO:86); mutagenesis (TTTCT to TTGCG at exon 14a) fwd GAGATAAAATACTCAGTTGCGTTCAGGGCCTGCCATCTATC (SEQ ID NO:87), and rev GATAGATGGCAGGCCCTGAACGCAACTGAGTATTTTATCTC (SEQ ID NO:88); mutagenesis (TTTCT to TTTCA at exon 14a) fwd GAGATAAAATACTCAGTTTCATTCAGGGCCTGCCATCTATC (SEQ ID NO:89) and rev GATAGATGGCAGGCCCTGAATGAAACTGAGTATTTTATCTC (SEQ ID NO:90); mutagenesis (TTTCT to TTTCG at exon 14a) fwd GAGATAAAATACTCAGTTTCGTTCAGGGCCTGCCATCTATC (SEQ ID NO:91), and rev GATAGATGGCAGGCCCTGAACGAAACTGAGTATTTTATCTC (SEQ ID NO:92); mutagenesis (TTTCT to TTTAT at exon 14a) fwd GAGATAAAATACTCAGTTTATTTCAGGGCCTGCCATCTATC (SEQ ID NO:93) and rev GATAGATGGCAGGCCCTGAAATAAACTGAGTATTTTATCTC (SEQ ID NO:94); and mutagenesis (branchpoint A to G) fwd GTCAAAGGGATATATTTTGGGATAAAATACTCAGTTTCTTTC (SEQ ID NO:95) and rev GAAAGAAACTGAGTATTTTATCCCAAAATATATCCCTTTGAC (SEQ ID NO:96).

Lariat Sequencing

To map the branchpoints that were used when the BRD9 poison exon was included or excluded, RT-PCR was performed to amplify branchpoint-spanning fragments from lariat RNAs arising during normal (poison exon exclusion) or aberrant (poison exon inclusion) splicing of BRD9 pre-mRNA. In brief, SuperScript III reverse transcriptase (Invitrogen) and a primer complementary to the intronic sequences downstream of the 5′ splice sites were used to generate cDNA from lariat RNAs. Branchpoint-spanning fragments were then amplified from lariat RNAs by nested PCR with pairs of outer primers (with the RT primer being the reverse primer) and inner primers. The forward primers were complementary to sequences about 200-300 nucleotides upstream of the 3′ splice sites and the reverse primers were complementary to sequences downstream of the 5′ splice sites. The PCR products were cloned into the pGEM-T vector (Promega) and sequenced by Sanger sequencing.

Melanoma Transplant Model

Stably transduced Melan-a, MEL270 and MEL202 cells (1M cells) were resuspended with doxycycline-inducible shRNAs, cDNAs or sgRNAs in 100 μl of a 1:1 mix of medium and Matrigel (BD Biosciences), and subcutaneously and bilaterally injected the mix into the flanks of 7-week-old female CB17-SCID mice (Taconic). For doxycycline-regulated shRNA induction, doxycycline-containing diets (625 mg/kg diet, Envigo) were used. To assess tumour growth, at least five mice per group were injected for a total of ten tumours per group. No randomization or blinding was used in the analysis of tumour growth. Tumours were measured with callipers every seven days. Growth curves were visualized with Prism GraphPad 8.0. Tumour volume was calculated using the formula;

Volume=π(length)(width)(height)/6.

In Vitro Morpholino Transfection

To deliver morpholinos into cultured cell lines, the manufacturer's instruction (GeneTools) were followed. In brief, 6 μM Endo-Porter were used after adding morpholinos (final concentration of 10 μM). RNA and proteins were collected 48 h after delivery. Morpholino target sequence no. 3 was TAATGAGGCAAGTCCAGTCCCGCTT (SEQ ID NO:4); no. 6 was AAAGAGGGGATAATGAGGCAAGTCC (SEQ ID NO:5); and no. 7 was GGGATAATGAGGCAAGTCCAGTCCC (SEQ ID NO:6).

In Vivo Morpholino Treatment

Treatment with morpholinos was started when the tumour volume in mice reached 100-200 mm. Cohorts were treated intratumorally with 12.5 mg/kg scrambled or poison-exon-targeting Vivo-Morpholinos (AAAGAGGGGATAATGAGGCAAGTCC (SEQ ID NO:5), GeneTools) dissolved in 50 μl PBS, every 2 days for 8 doses in total. The mice were dissected 24 h after the final treatment. For the patient-derived xenograft model, patient-derived rectal melanoma cells (SF3B1^{R625C}) and UVM cells (SF3B1 wild type) were serially transplanted into SCID mice and treated similarly.

In Vivo Metastasis Model

For lung experimental metastasis, B16 and 92.1 melanoma cells retrovirally transduced with shRNAs targeting Renilla, BRD9 (no. 1 and no. 1) or Brd9 (no. 1 or no. 2) in MLS-E vector (sorted using GFP) were trypsinized, resuspended in PBS and then 0.4 M cells in 0.2 ml PBS were injected via the lateral tail vein using a 27-gauge needle. Mice were killed 14 days after injection and tissues were isolated and fixed in 4% paraformaldehyde (Thermo Fisher Scientific). For evaluation of metastatic colonization of the lung using 92.1 human UVM cells, the burden of metastatic cells was evaluated using GFP expression by flow cytometry as well as anti-GFP immunohistochemistry 14 days following tail-vein injection of 0.4 M cells into NOD-SCID Il2rg^{-/-} mice.

Histological Analysis

Tissues were fixed in 4% paraformaldehyde, processed routinely in alcohol and xylene, embedded in paraffin, sectioned at 5-μm thickness and stained with H&E. Immunohistochemistry was performed on a Leica Bond RX automated stainer (Leica Biosystems). Following heat-induced epitope retrieval at pH 6.0, the primary antibody against Ki67 (Vector VP-K451) was applied, followed by application of a polymer detection system (DS9800, Novocastra Bond Polymer Refine Detection, Leica Biosystems) in which the chromogen was 3,3-diaminobenzidine tetrachloride (DAB) and the counterstain was haematoxylin. Photomicrograph examination of all H&E and immunohistochemistry slides were performed using a Zeiss Axioskop imaging.

BRD9 Expression Correlates.

The cor.test (in R) was used to calculate Spearman's p and the P value associated with the correlation of BRD9 expression with the expression of each coding gene across all samples within each cohort from the TCGA. Analysis was restricted to coding genes that are not on the same chromosome arm as BRD9 (chromosome 5p) to remove potential confounding effects of local correlations. Coding genes with P<0.01 in at least 10 cancer types were ranked by their absolute mean value of p (computed across all TCGA cohorts) and classified as RNA-binding if they were annotated with the ‘RNA-binding’ Gene Ontology term (GO: 0003723).

BRD9 Alternative Splicing.

Potential NMD-targeted isoforms of BRD9 were identified as follows: the MISO v.2.0 alternative splicing annotation (Katz, Y., et al. Analysis and design of RNA sequencing experiments for identifying isoform regulation. Nat. Methods 7, 1009-1015 (2010)) were queried for exon skipping and competing splice site events within the BRD9 gene locus, restricted to those events with evidence of alternative splicing based on spliced junction reads (described in ‘Genome annotation, RNA-seq read mapping, and estimation of gene and isoform expression’), assigned open reading frames for the isoforms resulting from each alternative splicing event based on the BRD9 isoform with the longest open reading frame, and classified isoforms as predicted NMD substrates if they contained a termination codon >50 nt upstream of a splice junction.

Robust linear modelling of BRD9 expression on the basis of the identified alternatively spliced isoforms of BRD9 that are predicted NMD substrates was performed for each TCGA cohort with the rlm function in the MASS package in R. Relative expression of each isoform in each sample was estimated from RNA-seq data across all TCGA cohorts as described in ‘Genome annotation, RNA-seq read mapping, and estimation of gene and isoform expression’. A z-score normalization was performed across all samples for each isoform in each cohort before model fitting. The resulting coefficients from the fitted models were subsequently used to predict BRD9 expression from BRD9 NMD-targeted isoform expression.

RNA-Seq Library Preparation.

RNA-seq libraries were prepared from TRIzol-isolated (Thermo Fisher cat. no. 15596026) RNA using the Illumina TruSeq RNA Library Prep Kit v.2 (Illumina cat. no. RS-122-2001/2). K562 libraries were sequenced at MSKCC with 101-bp single-end reads. MEL270 libraries were sequenced by the FHCRC Genomics Shared Resource with 2×51-bp paired-end reads.

ChIP-Seq Library Preparation.

ChIP-seq libraries were prepared and sequenced as described in Michel, B. C. et al. Nat. Cell Biol. 20, 1410-1420 (2018), by the Molecular Biology Core Facilities at the Dana-Farber Cancer Institute with 75-bp single-end reads.

Genomic Analysis of SWI-SNF Complex Members from TCGA.

Mutational analysis of genes encoding members of the SWI-SNF complex was performed as previously described (Mashtalir, N., et al. Modular organization and assembly of SWI/SNF family chromatin remodeling complexes. Cell 175, 1272-1288.e1220 (2018)).

Genome Annotation, RNA-Seg Read Mapping, and Estimation of Gene and Isoform Expression.

RNA-seq reads were processed for gene expression and isoform ratio quantification as previously described (Dvinge, H. et al. Sample processing obscures cancer-specific alterations in leukemic transcriptomes. Proc. Natl Acad. Sci. USA 111, 16802-16807 (2014)). In brief, RNA-seq reads were aligned to the hg19/GRCh37 assembly of the human genome using a gene annotation created by merging the UCSC known Gene gene annotation (Dvinge, H. et al. Sample processing obscures cancer-specific alterations in leukemic transcriptomes. Proc. Natl Acad. Sci. USA 111, 16802-16807 (2014)), Ensembl v.71.1 gene annotation (Flicek, P. et al. Ensembl 2013. Nucleic Acids Res. 41, D48-D55 (2013)) and MISO v.2.0 isoform annotation (Katz, Y., Wang, E. T., Airoldi, E. M. & Burge, C. B. Analysis and design of RNA sequencing experiments for identifying isoform regulation. Nat. Methods 7, 1009-1015 (2010). Read alignment and expression estimation were performed with RSEM v.1.2.4 Li, B. & Dewey, C. N. RSEM: accurate transcript quantification from RNA-seq data with or without a reference genome. BMC Bioinformatics 12, 323 (2011)), Bowtie v.1.0.0 (Langmead, B., et al. Ultrafast and memory-efficient alignment of short DNA sequences to the human genome. Genome Biol. 10, R25 (2009)) and TopHat v.2.1.1 (Trapnell, C., et al. TopHat: discovering splice junctions with RNA-seq. Bioinformatics 25, 1105-1111 (2009)). Isoform ratios were quantified with MISO v.2.0 (Katz, Y., et al. Analysis and design of RNA sequencing experiments for identifying isoform regulation. Nat. Methods 7, 1009-1015 (2010)). Gene expression estimates were normalized by applying the trimmed mean of M values method (Robinson, M. D. & Oshlack, A. A scaling normalization method for differential expression analysis of RNA-seq data. Genome Biol. 11, R25 (2010)) to coding genes. Statistical tests for differential gene and isoform expression were performed for single-sample comparisons with Wagenmakers' Bayesian framework (Wagenmakers, E. J., et al. Bayesian hypothesis testing for psychologists: a tutorial on the Savage-Dickey method. Cognit. Psychol. 60, 158-189 (2010)) and for sample group comparisons with the Mann-Whitney U-test. RNAseq read-coverage plots represent reads normalized by the number of reads mapping to all coding genes in each sample (per million).

RNA-Seq Coverage Plots.

RNA-seq coverage plots were made using the UCSC Genome Browser (Kent, W. J. et al. The human genome browser at UCSC. Genome Res. 12, 996-1006 (2002)) and/or the ggplot2 package in R (Wickham, H. ggplot2: Elegant Graphics for Data Analysis (Springer, New York, 2016)). Repetitive elements were annotated by RepeatMasker (Smit, A., Hubley, R. & Green, P. RepeatMasker Open-4.0, www.repeatmasker.org (2013-2015)).

Cluster Analysis.

Unsupervised clustering of chronic lymphocytic leukaemia, myelodysplastic syndrome and UVM samples (FIG. 1A) was based on the 40 events that were differentially spliced in isogenic UVM (MEL270 cells) as well as myeloid leukaemia (K562 cells) cells expressing SF3B1^{K700E} versus wild-type SF3B1, restricted to the 30 of these events that had sufficient read coverage in all cohorts for clustering.

ChIP-Seg Data Analysis.

ChIP-seq reads were mapped to the genome by calling Bowtie v.1.0.0 (Langmead, B., Trapnell, C., Pop, M. & Salzberg, S. L. Ultrafast and memory-efficient alignment of short DNA sequences to the human genome. Genome Biol. 10, R25 (2009)) with the arguments ‘-v 2 -k 1 -m 1-best-strata’. Peaks were called using MACS2 v.2.1.1.20160309 (Zhang, Y. et al. Model-based analysis of ChIP-seq (MACS). Genome Biol. 9, R137 (2008)) against input control libraries with P<10-5 and subsequently filtered to remove peaks contained within ENCODE blacklisted regions (The ENCODE Project Consortium. An integrated encyclopedia of DNA elements in the human genome. Nature 489, 57-74 (2012)) and the mitochondrial genome. Subsequent data analysis was performed with Bioconductor in the R programming environment (Huber, W. et al. Orchestrating high-throughput genomic analysis with Bioconductor. Nat. Methods 12, 115-121 (2015)). Consensus peaks between samples were called using the soGGI package v.1.14.0 (Dharmalingam G. & Carroll, T. soGGi: Visualise ChIP-seq, MNase-seq and motif occurrence as aggregate plots summarised over grouped genomic intervals. R package version 1.14.0, //rdrr.io/bioc/soGGi/(2018)). Peaks were annotated using the ChIPseeker package v.1.18.0 (Yu, G., Wang, L. G. & He, Q. Y. ChIPseeker: an R/Bioconductor package for ChIP peak annotation, comparison and visualization. Bioinformatics 31, 2382-2383 (2015)). Potential transcription factor binding in a 300-nucleotide region around the centre of consensus peaks was scored using the TFBSTools package v.1.20.0 (Tan, G. & Lenhard, B. TFBSTools: an R/bioconductor package for transcription factor binding site analysis. Bioinformatics 32, 1555-1556 (2016)), with models taken from the HOCOMOCO v.11 human core collection (Kulakovskiy, I. V. et al. HOCOMOCO: towards a complete collection of transcription factor binding models for human and mouse via large-scale ChIP-Seq analysis. Nucleic Acids Res. 46, D252-D259 (2018)) and applied with a threshold of P<10-4. The highest scores for each consensus peak region were collated for each transcription factor. A two-sided Mann-Whitney U-test was used to assess the significance of the difference in scores between constitutive and sensitive peaks for each transcription factor.

Data Availability

RNA-seq and ChIP-seq data generated as part of this study were deposited in the Gene Expression Omnibus (accession number GSE124720). RNA-seq data from published studies were downloaded from CGHub (TCGA UVM (Robertson, A. G., et al. Integrative analysis identifies four molecular and clinical subsets in uveal melanoma. Cancer cell 32, 204-220.e215 (2017))), EMBL-EBI ArrayExpress (Illumina Human BodyMap 2.0: E-MTAB-513), the Gene Expression Omnibus (accession numbers GSE72790 and GSE114922 for chronic lymphocytic leukaemia (Darman, R. B. et al. Cancer-associated SF3B1 hotspot mutations induce cryptic 3′ splice site selection through use of a different branch point. Cell Rep. 13, 1033-1045 (2015)) and myelodysplastic syndromes (Pellagatti, A. et al. Impact of spliceosome mutations on RNA splicing in myelodysplasia: dysregulated genes/pathways and clinical associations. Blood 132, 1225-1240 (2018)), respectively), or directly obtained from the authors (for UVM (Alsafadi, S. et al. Nat. Commun. 7, 10615 (2016)).

### ILLUSTRATIVE EMBODIMENTS

Various non-limiting, illustrative embodiments are described in the numbered paragraphs set forth below.

1. A method for treating cancer, a pre-malignant disease, or a dysplastic disease in a subject, the method comprising administering a BRD9 activating therapy to the subject.

2. A method for treating cancer, a pre-malignant disease, or a dysplastic disease in a subject, the method comprising increasing non-canonical BAF formation in the subject.

3. The method of one of paragraphs 1 or 2, wherein the subject has been diagnosed with cancer, a pre-malignant disease, or a dysplastic disease.

4. The method of any one of paragraphs 1-3, wherein the cancer or disease is a SF3B1-mutant cancer or disease.

5. The method of any one of paragraphs 1-4, wherein the subject has and/or has been diagnosed with a cancer or disease selected from blood cancer, bladder cancer, uveal melanoma, cutaneous cancer, pancreatic cancer, breast cancer, prostate cancer, genitourinary cancer, myeloid cancer, lymphoid cancer, myelodysplastic syndrome, and pre-malignant myeloid disease.

6. The method of any one of paragraphs 1-5, wherein the subject has been determined to have a SF3B1 mutation.

7. The method of paragraph 6, wherein the SF3B1 mutation comprises E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M97IT, or M971V.

8. The method of any one of paragraphs 1, or 3-7, wherein the BRD9 activating therapy comprises administering a BRD9 polypeptide or nucleic acid encoding a BRD9 polypeptide.

9. The method of any one of paragraphs 1 or 3-7, wherein the BRD9 activating therapy comprises a therapy that activates transcription of the endogenous BRD9 gene.

10. The method of any one of paragraphs 1 or 3-7, wherein the BRD9 activating therapy comprises a splicing modifier.

11. The method of paragraph 10, wherein the splicing modifier comprises an antisense nucleic acid having base complementarity with a BRD9 splice site.

12. The method of paragraph 11, wherein the antisense molecule binds to the branch point, 5′ splice site, or 3′ splice site of the poison exon, exon 14a.

13. The method of paragraph 10, wherein the splicing modifier mutates the branch point, 5′ splice site, or 3′ splice site of the poison exon, exon 14a.

14. The method of paragraph 10, wherein the splicing modifier mutates the exonic splicing enhancer of the poison exon, exon 14a.

15. The method of any one of paragraphs 10-14, wherein the splicing modifier comprises a modified nucleic acid.

16. The method of paragraph 15, wherein the splicing modifier comprises a morpholino.

17. The method of any one of paragraphs 1-16, wherein the subject has previously been treated for the disorder.

18. The method of paragraph 17, wherein the subject has been determined to be non-responsive to the previous therapy.

19. The method of any one of paragraphs 1-18, wherein the cancer or disorder is classified as refractory or recurrent.

20. The method of any one of paragraphs 1-19, wherein the subject is a human.

21. The method of any one of paragraphs 1-20, wherein the method further comprises administration of an additional therapy.

22. The method of any one of paragraphs 1-21, wherein the BRD9 activating therapy is administered by intravenous injection.

23. The method of any one of paragraphs 1-22, wherein the BRD9 activating therapy reconstitutes ncBAF formation.

24. An antisense oligonucleotide comprising at least 10 contiguous nucleotides of a nucleic acid selected from SEQ ID NOS:4-6 and 8-12, or the complement thereof.

25. The antisense oligonucleotide of paragraph 24, wherein the antisense oligonucleotide comprises at least one modified nucleotide.

26. The antisense oligonucleotide of paragraph 24 or 25, wherein the antisense oligonucleotide comprises a morpholino.

27. The antisense oligonucleotide of any one of paragraphs 24-26, wherein the antisense oligonucleotide comprises a nucleic acid sequence with at least 90% sequence identity to a nucleic acid selected from: SEQ ID NOS:4-6 and 8-12, a complement of SEQ ID NOS:4-6 and 8-12, and a functional fragment of SEQ ID NOS:4-6 and 8-12.

28. A method for treating cancer, a pre-malignant disease, or a dysplastic disease in a subject, the method comprising administering an antisense oligonucleotide according to any one of paragraphs 24-27.

29. The method of paragraph 28, wherein the subject has been diagnosed with cancer, a pre-malignant disease, or a dysplastic disease.

30. The method of paragraph 28 or 29, wherein the cancer or disease is a SF3B1-mutant cancer or disease.

31. The method of any one of paragraphs 28-30, wherein the subject has and/or has been diagnosed with a cancer or disease selected from blood cancer, bladder cancer, uveal melanoma, cutaneous cancer, pancreatic cancer, breast cancer, prostate cancer, genitourinary cancer, myeloid cancer, lymphoid cancer, myelodysplastic syndrome, and pre-malignant myeloid disease.

32. The method of any one of paragraphs 28-31, wherein the subject has been determined to have a SF3B1 mutation.

33. The method of paragraph 32, wherein the SF3B1 mutation comprises E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M97IT, or M971V.

34. The method of any one of paragraphs 28-33, wherein the subject has previously been treated for the disorder.

35. The method of paragraph 34, wherein the subject has been determined to be non-responsive to the previous therapy.

36. The method of any one of paragraphs 28-35, wherein the cancer or disorder is classified as refractory or recurrent.

37. The method of any one of paragraphs 28-36, wherein the subject is a human.

38. The method of any one of paragraphs 28-37, wherein the method further comprises administration of an additional therapy.

39. The method of paragraph 38, wherein the additional therapy comprises a BRD9 polypeptide or nucleic acid encoding a BRD9 polypeptide.

40. The method of any one of paragraphs 28-39, wherein the antisense oligonucleotide and/or additional therapy reconstitutes ncBAF formation and/or prevents or reduces BRD9 mis-splicing.

41. A method of increasing functional expression of BRD9 in a cell, comprising contacting the cell with an effective amount of a BRD9 activating agent.

42. The method of paragraph 41, wherein increasing functional expression of BRD9 in the cell results in increased non-canonical BAF formation in the cell.

43. The method of any one of paragraphs 41 or 42, wherein the cell is a transformed cell.

44. The method of paragraph 43, wherein the transformed cell is a cancer cell selected from a blood cancer, bladder cancer, uveal melanoma, cutaneous cancer, pancreatic cancer, breast cancer, prostate cancer, genitourinary cancer, myeloid cancer, and lymphoid cancer.

45. The method of any one of paragraphs 1-44, wherein the cell comprises a SF3B1 mutation.

46. The method of paragraph 45, wherein the SF3B1 mutation comprises E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, or M971V.

47. The method of any one of paragraphs 41-46, wherein the BRD9 activating agent comprises a BRD9 polypeptide or nucleic acid encoding a BRD9 polypeptide.

48. The method of any one of paragraphs 41-46, wherein the BRD9 activating agent comprises a splicing modifier.

49. The method of paragraph 48, wherein the splicing modifier comprises an antisense nucleic acid having base complementarity with a BRD9 splice site.

50. The method of paragraph 49, wherein the antisense molecule binds to the branch point, 5′ splice site, or 3′ splice site of the poison exon, exon 14a.

51. The method of paragraph 48, wherein the splicing modifier mutates the branch point, 5′ splice site, or 3′ splice site of the poison exon, exon 14a.

52. The method of paragraph 48, wherein the splicing modifier mutates the exonic splicing enhancer of the poison exon, exon 14a.

53. The method of any one of paragraphs 48-52, wherein the splicing modifier comprises a modified nucleic acid.

54. The method of paragraph 53, wherein the splicing modifier comprises a morpholino.

55. The method of any one of paragraphs 41-54, wherein the cell is in vivo in a subject with a treating cancer, a pre-malignant disease, or a dysplastic disease.

56. The method of paragraph 55, wherein the subject is a human.

57. The method of paragraph any one of paragraphs 41-54, wherein the cell is in vitro and/or ex vivo.

While illustrative embodiments have been illustrated and described, it will be appreciated that various changes can be made therein without departing from the spirit and scope of the invention.

1. A method for treating cancer, a pre-malignant disease, or a dysplastic disease in a subject in need thereof, the method comprising administering a BRD9 activating therapy to the subject.

2. (canceled)

3. (canceled)

4. The method of claim 1, wherein the cancer or disease is associated with a SF3B1 mutation.

5. (canceled)

6. (canceled)

7. The method of claim 4, wherein the SF3B1 mutation comprises E592K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M971T, or M971V.

8. The method of claim 1, wherein the BRD9 activating therapy comprises administering a BRD9 polypeptide a nucleic acid encoding a BRD9 polypeptide, or an agent that activates transcription of the endogenous BRD9 gene.

9. (canceled)

10. The method of claim 1, wherein the BRD9 activating therapy comprises a splicing modifier.

11. The method of claim 10, wherein the splicing modifier comprises an antisense nucleic acid or a morpholino having base complementarity with a BRD9 splice site.

12. The method of claim 11, wherein the antisense nucleic acid or a morpholino binds to or mutates the branch point, 5′ splice site, 3′ splice site, or exonic splicing enhancer of the poison exon, exon 14a.

13-16. (canceled)

17. The method of claim 1, wherein the subject has previously been treated for the cancer or disease, and the subject has been determined to be non-responsive to the previous therapy.

18. (canceled)

19. The method of claim 1, wherein the cancer or disorder is refractory or recurrent.

20. (canceled)

21. (canceled)

22. The method of claim 1, wherein the BRD9 activating therapy is administered by intravenous injection.

23. The method of claim 1, wherein the BRD9 activating therapy reconstitutes ncBAF formation, or prevents or reduces BRD9 mis-splicing.

24. An antisense oligonucleotide comprising at least 10 contiguous nucleotides of a nucleic acid complementary to a nucleic acid selected from SEQ ID NOS:4-6 and 8-12.

25. The antisense oligonucleotide of claim 24, wherein the antisense oligonucleotide comprises at least one modified nucleotide or a morpholino.

26. (canceled)

27. The antisense oligonucleotide of claim 24, wherein the antisense oligonucleotide comprises a nucleic acid sequence with at least 90% sequence identity to a nucleic acid complementary to a nucleic acid selected from: SEQ ID NOs:4-6 and 8-12, or a fragment thereof.

28-40. (canceled)

41. A method of increasing functional expression of BRD9 in a cell, comprising contacting the cell with an effective amount of a BRD9 activating agent.

42. The method of claim 41, wherein effective amount of a BRD9 activating agent increases non-canonical BAF formation in the cell.

43. (canceled)

44. The method of claim 41, wherein the cell is a cancer cell selected from a blood cancer, bladder cancer, uveal melanoma, cutaneous cancer, pancreatic cancer, breast cancer, prostate cancer, genitourinary cancer, myeloid cancer, and lymphoid cancer.

45. The method of claim 41, wherein the cell comprises a SF3B1 mutation selected from E892K, E622D, E622Q, E622V, Y623C, R625C, R625G, R625H, R625L, N626D, N626S, N626Y, A633V, H662Q, H662R, T663P, K666E, K666M, K666N, K666Q, K666R, K666T, K700E, V701F, R702Q, I704F, G740E, G742D, A762V, Y765C, D781E, D781G, M784I, E802Q, M9711T, or M971V.

46. (canceled)

47. The method of claim 41, wherein the BRD9 activating agent comprises a BRD9 polypeptide, nucleic acid encoding a BRD9 polypeptide, or a splicing modifier.

48. (canceled)

49. The method of claim 47, wherein the splicing modifier comprises an antisense nucleic acid or a morpholino having base complementarity with a BRD9 splice site, wherein the antisense molecule binds to or mutates the branch point, 5′ splice site, 3′ splice site, or exonic splicing enhancer of the poison exon, exon 14.

50-57. (canceled)
